(19) United States
(12) Patent Application Publication
(10) Pub. No.: US 2025/0122499 A1
(43) Pub. Date: Apr. 17, 2025

(54) ARTIFICIAL NUCLEIC ACIDS FOR RNA EDITING

(71) Applicant: Eberhard Karls Universität Tübingen, Tübingen (DE)

(72) Inventors: Philipp Reautschnig, Tübingen (DE); Jacqueline Wettengel, Tübingen (DE); Thorsten Stafforst, Tübingen (DE); Dr. Nicolai Wahn, Tübingen (DE)

(73) Assignee: Eberhard Karls Universität Tübingen, Tübingen (DE)

(21) Appl. No.: 18/031,555

(22) PCT Filed: Oct. 12, 2021
(86) PCT No.: PCT/EP2021/078126

§ 371 Date: Apr. 12, 2023

Related U.S. Application Data
(63) Continuation of application No. PCT/EP2020/078626, filed on Oct. 12, 2020.

(51) Int. Cl. C12N 15/113

(52) U.S. Cl. CPC C12N 15/113; C12N 2310/11

(57) ABSTRACT

The present invention provides an artificial nucleic acid for site-directed editing of a target RNA with enhanced editing specificity and avoiding undesirable off-target editing. The artificial nucleic acid comprises a targeting sequence comprising a nucleic acid sequence complementary to or at least partially complementary to a target sequence in the target RNA comprising one or more nucleotides to be edited, wherein the targeting sequence is flanked by a first recruiting moiety capable of recruiting a deaminase, and a second recruiting moiety capable of recruiting a deaminase, wherein at least one of the first and second recruiting moiety comprises at least one recruitment sequence, and preferably comprises at least two recruitment sequences, which bind(s) to complementary region(s) in the target RNA.

The present invention concerns artificial nucleic acids for site-directed editing of a target RNA. In particular, the present invention provides artificial nucleic acids capable of site-directed editing of endogenous transcripts by harnessing an endogenous deaminase. Further, the present invention provides artificial nucleic acids for sited-directed editing of a target RNA, which provide for enhanced editing specificity and avoid undesirable off-target editing. The invention also comprises a method for creating said artificial nucleic acid, wherein at least some steps of the method are computer assisted or computer implemented. Furthermore, the present invention provides a vector encoding said artificial nucleic acid, as well as a cell, a composition and a kit comprising said artificial nucleic acid. Moreover, the invention provides the use of the artificial nucleic acid, the vector, the cell, the composition or the kit for site-directed editing of a target RNA or for in vitro diagnosis. In addition, the artificial nucleic acid, the vector, the cell, the composition or the kit of the present invention are provided for use as a medicament or for use in diagnosis of a disease or disorder.

### BACKGROUND OF THE INVENTION

In conventional gene therapy, the genetic information is typically manipulated at the DNA level, thus permanently altering the genome. Depending on the application, the persistent modification of the genome may be either advantageous or imply serious risks. In this respect, the targeting of RNA instead of DNA represents an attractive alternative approach. When treating a subject on the RNA level, the change in gene expression is usually reversible, tunable and very frequently also more efficient. On the one hand, the limited duration of the effect will also limit the risks related to harmful side-effects. In addition, the possibility to finely tune the effect allows for continuously adjusting the therapy and to control the adverse effects in a time and dose-dependent manner. Furthermore, many manipulations of gene expression are not feasible or ineffective at the genome level, e.g. when the gene loss is either lethal or readily compensated by redundant processes. For example, it appears particularly attractive to target signaling networks at the RNA level. Many signaling cues are either essential, or they are strongly redundant so that a knockout sometimes does not result in a clear phenotype while a knockdown does.

Accordingly, there is an increasing interest in the engineering of RNA targeting strategies. One such strategy is RNA editing. (A)denosine-to-(I)nosine RNA editing is a natural enzymatic mechanism to diversify the transcriptome. Since inosine is biochemically interpreted as guanosine, A-to-I editing formally introduces A-to-G mutations, which can result in the recoding of amino acid codons, START and STOP codons, alteration of splicing, and alteration of miRNA activity, amongst others. Targeting such enzyme activities to specific sites at selected transcripts, a strategy called site-directed RNA editing, holds great promise for the treatment of disease and the general study of protein and RNA function. RNA editing strategies based on engineered deaminases were developed (see, for example, Vogel, P., Schneider, M. F., Wettengel, J., Stafforst, T. Improving Site-Directed RNA Editing In Vitro and in Cell Culture by Chemical Modification of the GuideRNA. Angew. Chem. Int. Ed. 53, 6267-6271 (2014). However, in a therapeutic setting, the harnessing of the widely expressed, endogenous deaminases acting on RNA would be the most attractive. It would allow for introducing a specific mutation into the transcriptome by administration of an oligonucleotide drug only, without the need for the ectopic expression of any (engineered) protein. For instance, Wettengel et al. (Wettengel, J., Reautschnig, J., Geisler, S., Kahle, P. J., Stafforst, T.: Harnessing human ADAR2 for RNA repair—Recoding a PINK1 mutation rescues mitophagy. Nucl. Acids Res. 45, 2797-2808 (2017)) reported a system that requires no artificial protein, but employs cellular ADAR2. Moreover, oligonucleotide constructs for site-directed RNA editing are described in international patent applications WO 2016/097212 and WO 2017/010556. Also German patent DE 10 2015 012 522 B3 describes a guideRNA molecule for site-directed RNA editing.

Qu et al. (Liang Qu et al.: Programmable RNA editing by recruiting endogenous ADAR using engineered RNAs. Nature Biotechnology, 37, 133-138 (2019)) constructed long unstructured gRNAs that recruit human wild-type ADARs to some extent, but have massive problems with bystander off-target editing.

Cox, et al. (Cox D. B. T., et al.: RNA editing with CRISPR-Cas13. Science 358, 1019-1027 (2017)) constructed an ADAR-recruiting RNA by fusing the deaminase domain of the hyperactive E1008Q mutant ADAR1 to an RNA-guided RNA-targeting CRISP effector.

However, the strategies known in the art suffer from similar problems: on the one hand, it proved difficult to recruit deaminase, in particular endogenous deaminase, efficiently enough in order to provide for sufficient RNA editing. On the other hand, efficient editing typically comes along with low specificity, e.g. numerous bystander off-target editings within the gRNA-mRNA duplex as well as global off-target editings all over the transcriptome. In addition, the strategies known in the art have an extremely limited sequence-space, as their gRNAs bind simply reverse-complementary around the target adenosine within target mRNA.

Merkle et al. (Merkle, T. Merz, S., Reautschnig, J., Blaha, A., Li, Q., Vogel, P., Wettengel, J., Li, J., Stafforst, T.: Precise RNA editing by recruiting endogenous ADARs with antisense oligonucleotides. Nature Biotechnology, 37, 1059-1069 (2019)) describe the construction of chemically modified antisense oligonucleotides (guideRNAs) that recruit endogenous human ADARs to edit endogenous transcripts in a more specific way. However, due to the chemically modifications, the antisense oligonucleotides used in this approach have to be administered to a subject because they cannot be synthesized by the organism itself.

There is therefore an urgent need of RNA editing strategies that allow for high editing yields and high specificity which do not result in off-target editing. In particular, compounds are required that are suitable for recruiting endogenous deaminases and which do not have to be added, e.g. by injection, to an individual but can be expressed by the individual itself based on vectors encoding the guide nucleic acids.

It is thus an objective of the present invention to provide a compound that is capable of recruiting a deaminase, preferably an endogenous deaminase, e.g. an adenosine deaminase, to an RNA target to be edited. A particular objective of the present invention is the provision of a compound suitable for editing an RNA target with high efficiency and high specificity, in particular with a reduced rate of off-target editing. Improved RNA editing approaches shall thus be provided, which allow for high yields of RNA editing at a specifically targeted site in a target RNA, preferably without or with reduced unspecific editing at other transcriptomic sites. Another particular objective of the present invention is the provision of a compound that is capable of recruiting a deaminase, preferably characterized by the afore-mentioned advantages, which can endogenously be expressed by an organism itself.

The solution of said object is achieved by the embodiments described herein and defined by the claims.

### DETAILED DESCRIPTION OF THE INVENTION

In a first aspect, the present invention concerns novel artificial nucleic acids for site-directed editing of a target RNA. In particular, an artificial nucleic acid for site-directed editing of a target RNA is provided herein, the artificial nucleic acid comprising in 5′ to 3′ direction or 3′ to 5′ direction:
a) a first recruiting moiety capable of recruiting a deaminase, wherein the first recruiting moiety comprises at least one recruitment sequence, which binds to a first region in the target RNA;
b) a targeting sequence which comprises a nucleic acid sequence complementary or at least partially complementary to a target sequence in the target RNA comprising one or more nucleotides to be edited, and
c) a second recruiting moiety capable of recruiting a deaminase, wherein the first region in the target RNA and the target sequence in the target RNA are separated by at least one nucleotide, which is not bound by the at least one recruitment sequence and which is not complementary to the targeting sequence of the artificial nucleic acid.

The inventors surprisingly found that the artificial nucleic acids as described herein, in particular an artificial nucleic acid comprising a targeting sequence which is flanked by two recruiting moieties, wherein at least one of the recruiting moieties comprises at least one recruitment sequence or a cluster of recruitment sequences, as defined herein, are capable of recruiting deaminases, particularly endogenous deaminases, to an RNA target and to specifically edit a nucleotide, preferably an adenosine or a cytidine nucleotide, at a target site in said RNA. Advantageously, the target RNA is edited by the artificial nucleic acid described herein with high efficiency, thus providing for high yields of edited target RNA while undesired off-target editing can be avoided. The artificial nucleic acid described herein thus allows for site-directed RNA editing with both, high efficiency as well as high specificity and opens a large sequence space of guideRNAs with advantageous properties over the state-of-art solutions.

The inventors have found that the artificial nucleic acid is suitable for editing a wide variety of transcripts, e.g. endogenous mRNAs of housekeeping genes (such as NUP43, GUSB, PDE4D) as well as plasmid encoded cDNA transcripts of disease-related genes (such as BMPR2 or COL3A1). Advantageously, the system according to the present invention proved to be applicable to a large variety of cells, ranging from immortalized cell lines and tumour cell lines to several primary human cells. For example, using the system of the present invention, the inventors successfully corrected the disease relevant hIDUA W402X amber mutation in primary fibroblasts from a Hurler syndrome patient. The use of the multivalent recruitment clusters opens a large sequence-space to design optimal guideRNAs for the targeting of any arbitrary endogenous (m)RNA. Accordingly, the inventors found recruitment sequences effective even when they are binding to intronic or exonic sequences thousands of nucleotides apart from each other on the targeted (m)RNA.

As used herein, the phrase ‘artificial nucleic acid (molecule)’ typically refers to a nucleic acid that does not occur naturally. In other words, an artificial nucleic acid molecule may be a non-natural nucleic acid. Such an artificial nucleic acid molecule may be non-natural due to its individual sequence (which does not occur naturally) and/or due to other modifications, e.g. structural modifications of nucleotides, which do not occur naturally in that context. An artificial nucleic acid as used herein preferably differs from a naturally occurring nucleic acid by at least one nucleotide or by at least one modification of a nucleotide. An artificial nucleic acid may be a DNA molecule, an RNA molecule or a hybrid-molecule comprising DNA and RNA portions. In preferred embodiments, the artificial nucleic acid is an RNA molecule. In particular, an artificial nucleic acid as used herein may comprise unmodified or modified ribonucleotides and/or unmodified or modified deoxynucleotides and preferably comprises unmodified ribonucleotides and/or deoxynucleotides. Further, the phrase ‘artificial nucleic acid (molecule)’ is not restricted to ‘one single molecule’ but may also refer to an ensemble of identical molecules. Accordingly, the phrase may refer to a plurality of identical molecules contained, for example, in a sample.

In the context of the present invention, the phrase ‘RNA editing’ refers to the reaction, by which a nucleotide, preferably an adenosine or a cytidine nucleotide, in a target RNA is transformed by a deamination reaction into another nucleotide. That change typically results in a different gene product, since the changed nucleotide preferably results in a codon change, leading e.g. to incorporation of another amino acid in the polypeptide translated from the RNA or to the generation or deletion of a stop codon. In particular, an adenosine nucleotide in a target RNA is converted to inosine by deamination, e.g. by an adenosine deaminase as described herein. In an alternative embodiment, a cytidine nucleotide in a target RNA is converted to a uridine nucleotide. As used herein, the term ‘target RNA’ typically refers to an RNA, which is subject to the editing reaction, which is supported by the artificial nucleic acid described herein.

The RNA editing achieved by the artificial nucleic acid described herein is further ‘site-directed’, which means that a specific nucleotide at a target site in a target RNA is edited, preferably without or essentially without editing other nucleotides. Typically, the nucleotide at the target site is targeted by the targeting sequence of the artificial nucleic acid described herein, wherein the targeting sequence is capable of specific base-pairing with the target sequence, preferably under physiological conditions. In the context of the present invention, the phrase ‘target sequence’ is thus typically used with regard to the nucleic acid sequence, which is (at least partially) complementary to the targeting sequence of the artificial nucleic acid. The target sequence comprises the target site, wherein the target site is typically a nucleotide, preferably an adenosine or a cytidine nucleotide, to be edited. In some embodiments, a target site may comprise two or more nucleotides to be edited, wherein these nucleotides are preferably separated from each other by at least one, preferably two, other nucleotides. As used herein, the terms ‘complementary’ or ‘partially complementary’ preferably refer to nucleic acid sequences, which due to their complementary nucleotides are capable of specific intermolecular base-pairing, preferably Watson-Crick and/or wobble base pairing, preferably under physiological conditions. The term ‘complementary’ as used herein may also refer to reverse complementary sequences. The artificial nucleic acid described herein may also be referred to herein as ‘antisense oligonucleotide’ or ‘ASO’, as the artificial nucleic acid typically comprises a nucleic acid sequence in the targeting sequence, which represents the antisense of a nucleic acid sequence in the target RNA. In the context of the present invention, the term ‘guideRNA’ may also be used in order to refer to the artificial nucleic acid, which preferably guides the deaminase function to the target site.

An artificial nucleic acid for site-directed editing of a target RNA is provided herein, the artificial nucleic acid comprising in 5′ to 3′ direction or 3′ to 5′ direction:
a) a first recruiting moiety capable of recruiting a deaminase, wherein the first recruiting moiety comprises at least one recruitment sequence, which binds to a first region in the target RNA;
b) a targeting sequence which comprises a nucleic acid sequence complementary or at least partially complementary to a target sequence in the target RNA comprising one or more nucleotides to be edited, and
c) a second recruiting moiety capable of recruiting a deaminase.

In this context, the second recruiting moiety capable of recruiting a deaminase preferably comprises a nucleic acid sequence capable of binding a deaminase, preferably a nucleic acid sequence as defined herein, such as an R/G motif.

### Recruiting Moieties

The artificial nucleic acid of the present invention comprises at least two recruiting moieties flanking a targeting sequence. In the context of the present invention, the term ‘recruiting moiety’ refers to a moiety of the artificial nucleic acid described herein, which recruits the deaminase and which is typically covalently linked to the targeting sequence. Such a ‘recruiting moiety’ thus recruits a deaminase to the target site in a target RNA, wherein the target RNA (and the target site) are preferably recognized and bound in a sequence-specific manner by the targeting sequence and at least one recruiting moiety.

The artificial nucleic acid of the present invention comprises at least two recruiting moieties, a first recruiting moiety and a second recruiting moiety, wherein at least one of the first and the second recruiting moiety is located 3′ of the targeting sequence and at least one of the first and second recruiting moieties is located 5′ of the targeting sequence.

It has to be noted that the terms ‘first recruiting moiety’ and ‘second recruiting moiety’ are to distinguish the recruiting moieties of the artificial nucleic acid which are located 3′ and 5′ of the targeting sequence, without implying any restriction as to the nature of the first and second recruiting moieties. In other words, the features described with respect to the ‘first’ recruiting moiety can also apply to the ‘second’ recruiting moiety and vice versa.

### First Recruiting Moiety

In the artificial nucleic acid of the present invention, the first recruiting moiety comprises at least one recruitment sequence which binds to a first region in the target RNA. In a preferred embodiment, the at least one recruitment sequence of the artificial nucleic acid comprises a nucleic acid sequence complementary or at least partially complementary to a first region in the target RNA. The recruitment sequence of the first recruiting moiety, together with the targeting sequence, thus directs the deaminase towards the target site in a target RNA in a sequence-specific manner.

In a further preferred embodiment of the present invention, the first recruiting moiety comprises a cluster of recruitment sequences comprising at least two recruitment sequences which are linked via a nucleotide linker. Preferably, the cluster comprises at least three recruitment sequences, for example 3 to 20 recruitment sequences, e.g. 3, 4, 5, 6, 7, 8, 9, 10, 11, 12, 13, 14, 15, 16, 17, 18, 19 or 20 recruitment sequences, and more preferably comprises 3 to 10 recruitment sequences.

The recruitment sequence, and more preferably the cluster of recruitment sequences increases the binding affinity of the artificial nucleic acid molecule to the target RNA compared to a guide nucleic acid which is complementary to the target RNA only in the target region comprising the nucleotide to be edited. Further, the provision of multiple recruitment sequences complementary or at least partially complementary to multiple regions in the target RNA also may increase the probability of encounters of artificial nucleic acid and target RNA.

Two recruitment sequences included in the cluster of recruitment sequences are either positioned immediately next to each other or linked via a nucleotide linker. In other terms an optional nucleotide linker may preferably separate two recruitment sequences in the cluster of recruitment sequences. The nucleotide linker linking the recruitment sequences comprises at least 1 nucleotide which preferably does not bind to or is not complementary to regions in the target RNA, which are bound by or which are complementary to the (at least two) recruitment sequences of the artificial nucleic acid. The nucleotide linker linking the (at least two) recruitment sequences of the first recruiting moiety of the artificial nucleic acid may comprise any type of nucleotide, such as e.g. adenosine, guanosine, cytidine, uridine or thymidine nucleotides, and preferably comprises (an) adenosine nucleotide(s). The nucleotide linker linking the recruitment sequences may e.g. comprise 1 to 100 nucleotides, e.g. 1 to 90, 1 to 80, 1 to 70, 1 to 60, 1 to 50, 1 to 40, 1 to 30, 1 to 20 or 1 to 10 nucleotides, and preferably comprises 2 to 6 nucleotides, which preferably are adenosine nucleotides.

Upon binding of the artificial nucleic acid to the target RNA, the nucleotide linker linking two recruitment sequences in the artificial nucleic acid thus “bridges” the nucleotides of the target RNA which are not complementary to and therefore are not bound by the recruitment sequences. However, it has to be noted that the number of nucleotides located between two regions on the target RNA which are bound by two individual recruitment sequences generally does not correspond to the number of nucleotides of the nucleotide linker linking the recruitment sequences in the artificial nucleic acid. For example, the distance between two regions on the target RNA serving as binding sites for two adjacent recruitment sequences of the artificial nucleic acid may comprise up to several hundred or even several thousand nucleotides, while the two adjacent recruitment sequences are separated by a nucleotide linker, which is considerably shorter, preferably as defined herein. In this particular case, in the course of binding of the recruitment sequences to the complementary regions of the target RNA, the regions of the target RNA located between two specific binding sites of the recruitment sequences may form a loop in the target RNA. The feasibility to use recruitment sequences in such vast distances to each other (large sequence space) highlights the flexibility of the present invention compared to the state of the art.

The at least one recruitment sequence of the first recruiting moiety may comprise any number of nucleotides which bind and preferably are complementary to the nucleotides of a first region in the target RNA. Preferably, the (at least one) recruitment sequence comprises at least 10, preferably at least 15, more preferably at least 20 nucleotides. In a preferred embodiment of the present invention, the at least one recruitment sequence comprises 10 to 200 nucleotides, and more preferably comprises 10 to 100 nucleotides or 15 to 100 nucleotides or 20 to 100 nucleotides. Preferably, the at least one recruitment sequence of the first recruiting moiety is present as an essentially single-stranded nucleic acid, in particular under physiological conditions.

As mentioned above, in a preferred embodiment, the first recruiting moiety of the artificial nucleic acid comprises a cluster of recruitment sequences comprising at least two recruitment sequences. Therefore, in a preferred embodiment, a first recruitment sequence binds to a first region in the target RNA and preferably comprises a nucleic acid sequence complementary or at least partially complementary to the first region in the target RNA, and a second or further recruitment sequence binds to a second or further region in the target RNA and preferably comprises a nucleic acid sequence complementary or at least partially complementary to the second or further region in the target RNA. For example, in a particular embodiment, the first recruiting moiety comprises three recruitment sequences, wherein a first recruitment sequence binds to a first region in the target RNA and preferably comprises a nucleic acid sequence complementary or at least partially complementary to the first region in the target RNA, and a second recruitment sequence binds to a second region in the target RNA and preferably comprises a nucleic acid sequence complementary or at least partially complementary to the second or further region in the target RNA, and a third recruitment sequence binds to a third region in the target RNA and preferably comprises a nucleic acid sequence complementary or at least partially complementary to the third region in the target RNA.

The plurality of recruitment sequences forming a cluster of recruitment sequences in the first recruiting moiety of the artificial nucleic acid thus guides, together with the targeting sequence, the deaminase to the target site in a sequence specific manner thereby increasing the binding affinity of the artificial nucleic acid to the target RNA. Due to the large sequence space that is available for each recruitment sequence their selection is very flexible and thus will allow to optimize various key properties of the guideRNA including editing efficiency, specificity, bystander editing, but also stability, immunogenicity, toxicity, and others.

Preferably, the first region in the target RNA which is bound by the first recruitment sequence of the first recruiting moiety, and/or the second and/or further region in the target RNA which is/are bound by a second and/or further recruitment sequence of the first recruiting moiety, does not comprise any editable adenosine nucleotide(s). That is, the first and further regions of the target sequence do preferably not comprise any adenosine nucleotides except for adenosine nucleotides in a specific codon context (e.g. adenosine nucleotides following a guanosine nucleotide in a 5′-GA-3′ context) or at a specific position (e.g. within 5 nt from the 5′ or 3′end of a recruitment sequence), which are subjected to reduced or even absent RNA editing.

Accordingly, the recruitment sequence(s) of the first recruiting moiety of the artificial nucleic acid is/are preferably depleted from uridine bases unless they are either within 5 nt from either end (5′ or 3′) of a recruitment sequence or in a 5′-NUS (S═C or G) context.

In certain embodiments of the invention, the recruitment sequence(s) of the first recruiting moiety of the artificial nucleic acid preferably has/have a reduced uridine content. For example, the recruitment sequence(s) of the first recruiting moiety preferably contain(s) not more than 50%, not more than 25%, not more than 20%, not more than 15%, not more than 10%, not more than 5%, not more than 4%, not more than 3%, not more than 2%, not more than 1%, or even less than 1% uridine nucleotides with respect to the total number of nucleotides contained in the recruitment sequence(s), unless they are either within 5 nt from either end (5′ or 3′) of a recruitment sequence or in a 5′-NUS (S═C or G) context. More preferably, the recruitment sequence(s) of the first recruiting moiety of the artificial nucleic acid does/do not contain any uridine bases, with the exception of uridine bases which are either within 5 nt from either end (5′ or 3′) of a recruitment sequence or in a 5′-NUS (S═C or G) context.

In this way, off-target editing events in the double-stranded region formed by the first and further regions of the target RNA and the recruitment sequence(s) are avoided.

In a preferred embodiment, the artificial nucleic acid comprises a nucleotide spacer between the first recruiting moiety and the targeting sequence. The nucleotide spacer preferably does not bind to or is not complementary to i) the (first) region in the target RNA which is bound by the recruitment sequence adjacent to the targeting sequence of the artificial nucleic acid, and ii) the target sequence in the target RNA.

The optional nucleotide spacer, which may be located between the first recruiting moiety and the targeting sequence of the artificial nucleic acid, may comprise any nucleotides, such as e.g. adenosine, guanosine, cytidine, uridine or thymidine nucleotides, and preferably comprises (an) adenosine nucleotide(s). As described herein with respect to the optional nucleotide linker linking the recruitment sequences of the first recruiting moiety, the nucleotide spacer optionally located between the first recruiting moiety and the targeting sequence preferably comprises at least one nucleotide, e.g. 1 to 100 nucleotides, more preferably 1 to 90, 1 to 80, 1 to 70, 1 to 60, 1 to 50, 1 to 40, 1 to 30, 1 to 20 or 1 to 10 nucleotides, and even more preferably comprises 2 to 6 nucleotides, which preferably are adenosine nucleotides.

The nucleotide spacer linking the targeting sequence and the first recruitment sequence in the artificial nucleic acid thus “bridges” the nucleotides of the target RNA which are located between the target sequence (bound by the targeting sequence of the artificial nucleic acid) and the first region in the target RNA (bound by the first recruitment sequence of the artificial nucleic acid). Again, it has to be noted that the number of nucleotides located between the target sequence and the first region on the target RNA which are bound by the targeting sequence and the first recruitment sequence, respectively, generally does not correspond to the number of nucleotides of the nucleotide spacer. Rather, the distance between the regions on the target RNA serving as binding sites for the targeting sequence and the first recruitment sequence of the artificial nucleic acid may comprise, for instance, up to several hundred or even several thousand nucleotides. In this particular case, in the course of binding of the targeting sequence and the first recruitment sequence to the complementary regions of the target RNA, the nucleotides located between the specific binding sites of the targeting sequence and the first recruitment sequence will form a loop in the target RNA.

### Targeting Sequence

The targeting sequence which is flanked by the first recruiting moiety and the second recruiting moiety of the artificial nucleic acid comprises a nucleic acid sequence complementary or at least partially complementary to a target sequence in the target RNA comprising one or more nucleotides to be edited. Preferably, the targeting sequence comprises a nucleic acid sequence complementary to or at least 60%, 70%, 80%, 90%, 95% or 99% complementary to a nucleic acid sequence in the target RNA, wherein the complementary nucleic acid sequence in the target RNA comprises the target site and preferably comprises at least 10 nucleotides, e.g. at least 12, at least 15, at least 18, at least 20, at least 22, at least 25 or at least 30 nucleotides. In a preferred embodiment, the targeting sequence comprises 10 to 50, more preferably 16 to 40 nucleotides. Preferably, the targeting sequence of the artificial nucleic acid is present as an essentially single-stranded nucleic acid, in particular under physiological conditions.

According to some embodiments, the targeting sequence of the artificial nucleic acid comprises at the position corresponding to a nucleotide to be edited in the target sequence a cytidine nucleotide or a variant thereof, a deoxycytidine nucleotide or a variant thereof, or an abasic site. Preferably, the targeting sequence comprises at the position corresponding to a nucleotide to be edited in the target sequence, preferably an adenosine to be edited, a cytidine nucleotide mismatching the adenosine to be edited. Preferably, the cytidine nucleotide mismatching the adenosine to be edited is positioned at least 6 nucleotides distant from either the 5′ or 3′ terminus of the targeting sequence. More preferably, the cytidine nucleotide mismatching the adenosine to be edited is positioned off-centre within the targeting sequence. For example, in a targeting sequence consisting of 20 nucleotides, the cytidine nucleotide mismatching the adenosine to be edited is positioned at position 8 with respect to the 3′ or 5′ terminus, preferably to the 3′ terminus, of the targeting sequence.

In some embodiments, the target site in the target RNA comprises two or more nucleotides to be edited, wherein these nucleotides are preferably separated from each other by at least one, preferably two, other nucleotides. In these embodiments, the targeting sequence may comprise at each position corresponding to a nucleotide to be edited a nucleotide as described above, preferably a cytidine nucleotide or a variant thereof.

The individual nucleic acid sequence of a targeting sequence of an artificial nucleic acid for editing a given target RNA typically depends on the sequence of the target site of a specific target RNA to be edited.

### Second Recruiting Moiety

In addition to the first recruiting moiety and the targeting sequence, the artificial nucleic acid of the present invention comprises a second recruiting moiety capable of recruiting a deaminase which is located 3′ or 5′ to the targeting sequence. Accordingly, the artificial nucleic acid may comprise the structure 5′—first recruiting moiety—targeting sequence—second recruiting moiety—3′ or 5′—second recruiting moiety—targeting sequence—first recruiting moiety—3′.

In an embodiment of the artificial nucleic acid, the second recruiting moiety may be a recruiting moiety as defined with respect to the first recruiting moiety. That is, the second recruiting moiety may comprise at least one recruitment sequence which binds to and preferably is complementary to or at least partially complementary to a specific region in the target RNA, and more preferably comprises a cluster of at least two recruitment sequences which are linked via an optional nucleotide linker, as defined above with respect to the first recruiting moiety. In this particular embodiment, the artificial nucleic acid may optionally comprise a nucleotide spacer between the targeting sequence and the second recruiting moiety, preferably a nucleotide spacer as defined with respect to the nucleotide spacer, which may be present between the targeting sequence and the first recruiting moiety.

However, in a preferred embodiment, the second recruiting moiety comprises a nucleic acid sequence capable of binding to a deaminase, preferably an adenosine deaminase, without binding to the target mRNA. That is, in a preferred embodiment of the artificial nucleic acid, the first recruiting moiety and the second recruiting moiety differ in their modes of recruiting a deaminase.

In certain embodiments, the second recruiting moiety of the artificial nucleic acid comprises or consists of at least one coupling agent capable of recruiting a deaminase, wherein the deaminase comprises a moiety that binds to said coupling agent. The coupling agent, which recruits a deaminase is typically covalently linked to the 5′-terminus or to the 3′-terminus of the targeting sequence. The coupling agent may alternatively also be linked to an internal nucleotide (i.e. not a 5′- or 3′-terminal nucleotide) of the targeting sequence, for example via linkage to a nucleotide variant or a modified nucleotide, preferably as described herein, such as amino-thymidine.

The coupling agent may be selected from the group consisting of O6-benzylguanine, O2-benzylcytosine, chloroalkane, 1×BG, 2×BG, 4×BG, and a variant of any of these. According to a particular embodiment, the coupling agent is a branched molecule, such as 2×BG or 4×BG, each of which is preferably capable of recruiting a deaminase molecule, thus preferably amplifying the editing reaction. Exemplary structures of suitable branched coupling agents are depicted below:

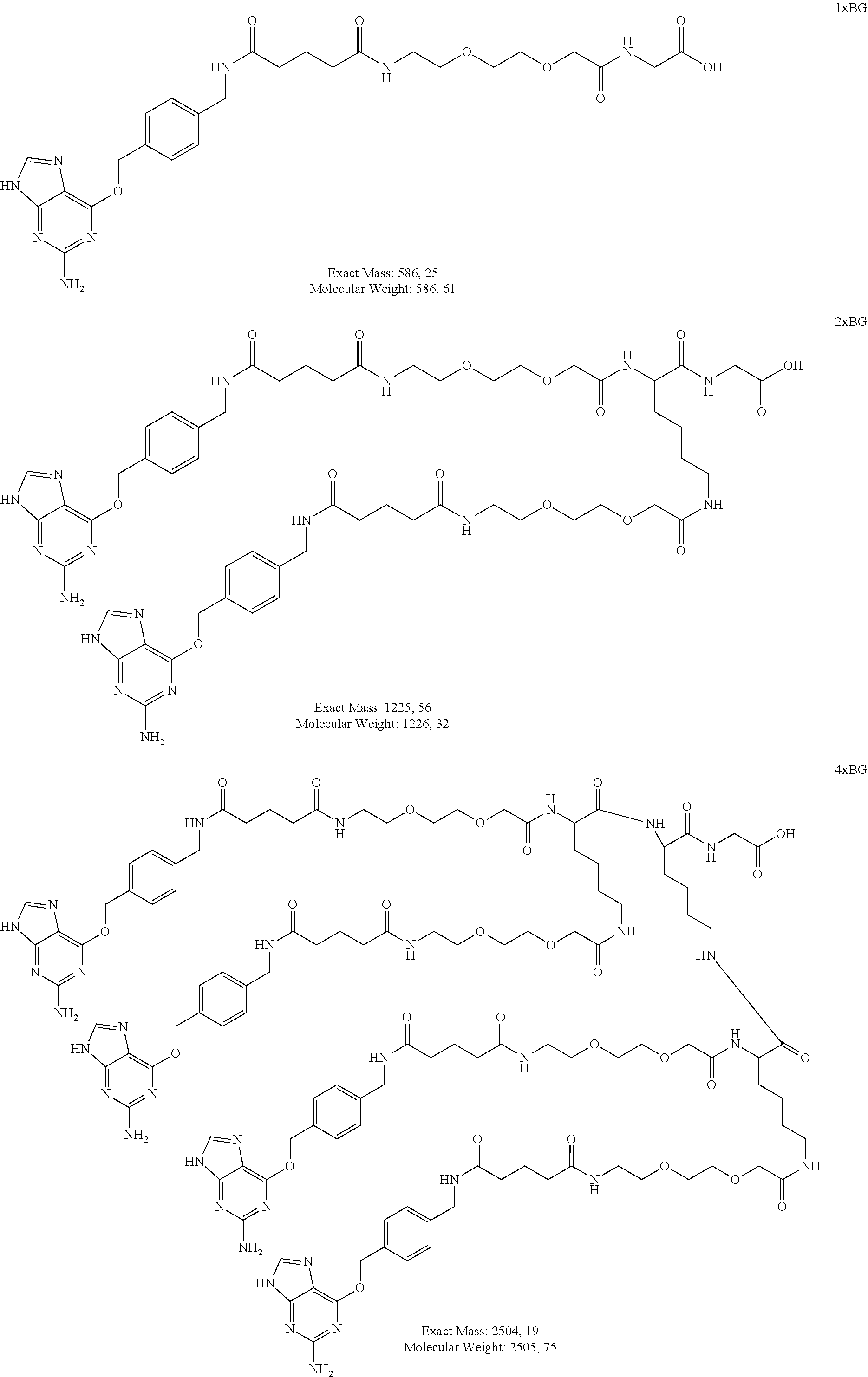 [SMILES: Nc1nc(OCc2ccc(CNC(=O)CCCC(=O)NCCOCCOCC(=O)NCC(=O)O)cc2)c2nc[nH]c2n1 ; Nc1nc(OCc2ccc(CNC(=O)CCCC(=O)NCCOCCOCC(=O)NCCCCC(NC(=O)COCCOCCNC(=O)CCCC(=O)NCc3ccc(COc4nc(N)nc5[nH]cnc45)cc3)C(=O)NCC(=O)O)cc2)c2nc[nH]c2n1 ; Nc1nc(OCc2ccc(CNC(=O)CCCC(=O)NCCOCCOCC(=O)NCCCCC(NC(=O)COCCOCCNC(=O)CCCC(=O)NCc3ccc(COc4nc(N)nc5[nH]cnc45)cc3)C(=O)CNC(CCCCNC(=O)C(CCCCNC(=O)COCCOCCNC(=O)CCCC(=O)NCc3ccc(COc4nc(N)nc5[nH]cnc45)cc3)NC(=O)COCCOCCNC(=O)CCCC(=O)NCc3ccc(COc4nc(N)nc5[nH]cnc45)cc3)C(=O)NCC(=O)O)cc2)c2nc[nH]c2n1]

The coupling agent is preferably capable of specifically binding to a moiety in a deaminase. Said moiety in a deaminase is preferably a tag, which is linked to a deaminase as described herein, preferably an adenosine deaminase or a cytidine deaminase as described herein. More preferably, said tag is selected from the group consisting of a SNAP-tag, a CLIP-tag, a HaloTag, and a fragment or variant of any one of these.

In the context of the present invention, a ‘variant’ of a nucleic acid sequence or of an amino acid sequence is at least 40%, preferably at least 50%, more preferably at least 60%, more preferably at least 70%, even more preferably at least 80%, even more preferably at least 90%, most preferably at least 95% identical to the sequence, the variant is derived from. Preferably, the variant is a functional variant.

As used herein, a ‘fragment’ of a nucleic acid sequence or of an amino acid sequence consists of a continuous stretch of nucleotides or amino acid residues corresponding to a continuous stretch of nucleotides or amino acid residues in the full-length sequence, which represents at least 5%, 10%, 20%, preferably at least 30%, more preferably at least 40%, more preferably at least 50%, even more preferably at least 60%, even more preferably at least 70%, even more preferably at least 80%, and most preferably at least 90% of the full-length sequence, the fragment is derived from. Such a fragment, in the sense of the present invention, is preferably a functional fragment.

Accordingly, the deaminases bound by the coupling agent in these embodiments are preferably artificial versions of endogenous deaminases, preferably of a deaminase as described herein. Preferably, the deaminase is selected from the group consisting of SNAP-ADAR1, SNAP-ADAR2, Apobec1-SNAP, SNAPf-ADAR1, SNAPf-ADAR2, Apobec1-SNAPf, Halo-ADAR1, Halo-ADAR2, Apobec1-Halo, Clip-ADAR1, Clip-ADAR2, Clipf-ADAR1, Clipf-ADAR2, Apobec1-Clip and Apobec1-Clipf, preferably as described herein, or a fragment or variant of any of these, wherein the deaminase is preferably derived from human, mouse or rat. More preferably, the deaminase is selected from the group consisting of SNAP-ADAR1, SNAP-ADAR2, SNAPf-ADAR1, SNAPf-ADAR2, Halo-ADAR1, Halo-ADAR2, Clip-ADAR1, Clip-ADAR2, Clipf-ADAR1 and Clipf-ADAR2, or a fragment or variant of any of these, wherein the deaminase is derived from human. According to another embodiment, the deaminase is selected from the group consisting of mApobec1-SNAP, mApobec1-SNAPf, mApobec1-Halo, mApobec1-Clip and mApobec1-Clipf, or a fragment or variant of any of these, wherein the deaminase is derived from mouse.

In a particularly preferred embodiment, the deaminase is a hyperactive mutant of any of the deaminases mentioned herein, preferably a hyperactive Q mutant, more preferably a hyperactive Q mutant of an ADAR1 deaminase, an ADAR2 deaminase (e.g. human ADAR1p150, E1008Q; human ADAR1p110, E713Q; human ADAR2, E488Q) or a tagged version thereof, most preferably as described herein, or a fragment or variant of any of these.

In another embodiment, the deaminase is a mutant of any of the deaminases mentioned herein that changes the nucleotide specificity of the deaminase from adenosine to another nucleotide e.g. an ADAR2 deaminase mutant that can perform C-to-U RNA editing (see Abudayyeh, O. O., et al.: A cytosine deaminase for programmable single-base RNA editing. Science 365(6451): 382-386. (2019)).

Tagged deaminases, preferably as described herein, (e.g. SNAP-, SNAPf-, Clip-, Clipf-, Halo-tagged deaminases or fragments or variants thereof) are preferably overexpressed for RNA editing, for example by transient transfection of a cell with a vector encoding said tagged deaminase or by stable expression in a transgenic cell, tissue or organism.

In certain embodiments, the second recruiting moiety of the artificial nucleic acid comprises or consists of at least one RNA motif (e.g. MS2-loop(s), direct repeats of trans-activating crRNA(s), BoxB motif(s), HIV trans-activation response (TAR) hairpin(s)) capable of recruiting a deaminase or another effector fusion-protein that was developed for a tethering approach, like MCP-ADAR (Azad, M. T. A., et al.: Site-directed RNA editing by adenosine deaminase acting on RNA for correction of the genetic code in gene therapy. Gene Ther 24(12): 779-786 (2017), and D. Katrekar et al.: In vivo RNA editing of point mutations via RNA-guided adenosine deaminases. Nat. Methods 16(3), 239-242 (2019)); or like dCas-ADAR (Cox, D. B. T., et al., supra; Omar O. Abudayyeh, et al., supra, or like LambdaN-ADAR (Montiel-Gonzalez, M. F., et al.: Correction of mutations within the cystic fibrosis transmembrane conductance regulator by site-directed RNA editing. Proc Natl Acad Sci USA 110(45): 18285-18290(2013)), or like TBP-ADAR (S. Rauch et al.: Programmable RNA-Guided RNA Effector Proteins Built from Human Parts. Cell 178, 122-134.e12 (2019)).

In preferred embodiments of the present invention, the second recruiting moiety comprises or consists of a nucleic acid sequence capable of specifically binding to a double-stranded (ds) RNA binding domain of a deaminase, preferably an adenosine or cytidine deaminase, more preferably an adenosine deaminase. Advantageously, the recruiting moiety comprising or consisting of a nucleic acid sequence capable of binding to a deaminase binds to endogenous deaminases. The artificial nucleic acid according to the invention thus promotes site-directed RNA editing employing an endogenous (or heterologously expressed) deaminase.

The artificial nucleic acid is suitable for site-directed editing of an RNA by a deaminase, wherein the deaminase is preferably an adenosine deaminase or a fragment or variant thereof, preferably an ADAR (adenosine deaminase acting on dsRNA) enzyme or a fragment or variant thereof, more preferably selected from the group consisting of ADAR1, ADAR2 and a fragment or variant thereof, e.g. a peptide or protein comprising an adenosine deaminase domain. However, also a cytidine deaminase or a fragment or variant thereof, e.g. Apobec1 or a fragment or variant thereof, e.g. a peptide or protein comprising a cytidine deaminase domain, are contemplated.

The term ‘deaminase’ as used herein refers to any peptide, protein or protein domain, which is capable of catalyzing the deamination of a nucleotide or a variant thereof in a target RNA, in particular the deamination of adenosine or cytidine. The term thus not only refers to full-length and wild type deaminases, such as ADAR1, ADAR2 or Apobec1, but also to a fragment or variant of a deaminase, preferably a functional fragment or a functional variant. In particular, the term also refers to mutants and variants of a deaminase, such as mutants of ADAR1, ADAR2 or Apobec1, preferably as described herein. Furthermore, the term deaminase as used herein also comprises any deaminase fusion protein (e.g. based on Cas9, Cas13, MS2 Coat Protein or the Lambda-N-peptide, TAR binding protein). In the context of the present invention, the term ‘deaminase’ also refers to tagged variants of a deaminase, such as a deaminase selected from the group consisting of SNAP-ADAR1, SNAP-ADAR2, Apobec1-SNAP, SNAPf-ADAR1, SNAPf-ADAR2, Apobec1-SNAPf, Halo-ADAR1, Halo-ADAR2, Apobec1-Halo, Clip-ADAR1, Clip-ADAR2, Clipf-ADAR1, Clipf-ADAR2, Apobec1-Clip and Apobec1-Clipf, or a fragment or variant of any of these, wherein the deaminase is preferably derived from human, mouse or rat.

In a preferred embodiment, the deaminase is an adenosine deaminase (such as ADAR1, preferably ADAR1p150 or ADAR1p110, or ADAR2), preferably a eukaryotic adenosine deaminase, more preferably a vertebrate adenosine deaminase, even more preferably a mammalian adenosine deaminase, most preferably a human adenosine deaminase, such as hADAR1 or hADAR2, or a fragment or variant of any of these.

According to an alternative embodiment, the deaminase is a cytidine deaminase (such as Apobec1, preferably human Apobec1, murine Apobec1 (mApobec1) or rat Apobec1 (rApobec1)), e.g. an eukaryotic cytidine deaminase, such as a vertebrate cytidine deaminase, preferably a mammalian cytidine deaminase, more preferably a murine or human cytidine deaminase, or a fragment or variant of any of these. The deaminase may be a tagged cytidine deaminase, or a fragment or variant thereof. The deaminase may be selected from the group consisting of mApobec1-SNAP, mApobec1-SNAPf, mApobec1-Halo, mApobec1-Clip and mApobec1-Clipf, or a fragment or variant of any of these, wherein the deaminase is derived from human, mouse or rat.

In an alternative embodiment, the deaminase is a hyperactive mutant of any of the deaminases mentioned herein, e.g. a hyperactive Q mutant, such as a hyperactive Q mutant of an ADAR1 deaminase, an ADAR2 deaminase (e.g. human ADAR1p150, E1008Q; human ADAR1p110, E713Q; human ADAR2, E488Q) or a tagged version thereof, or a fragment or variant of any of these.

Preferably, the second recruiting moiety comprises or consists of a nucleic acid sequence capable of specifically binding to a double stranded (ds) RNA binding domain of a deaminase, wherein the nucleic acid sequence is preferably linked covalently either to the 5′ terminus or to the 3′ terminus of the targeting sequence, more preferably to the 5′ terminus of the targeting sequence.

In preferred embodiments, the recruiting moiety comprises or consists of a nucleic acid sequence that is capable of intramolecular base pairing. The recruiting moiety preferably comprises or consists of a nucleic acid sequence that is capable of forming a stem-loop structure. In certain embodiments, said stem-loop structure comprises or consists of a double-helical stem comprising at least two mismatches. In a preferred embodiment, the stem loop structure comprises a loop consisting of from 3 to 8, preferably from 4 to 6, more preferably 5, nucleotides. The loop preferably comprises or consists of the nucleic acid sequence GCUAA or GCUCA.

In a preferred embodiment, the second recruiting moiety of the artificial nucleic acid comprises the nucleotide sequence 5′-GGUGU CGAGA AGAGG AGAAC AAUAU GCUAA AUGUU GUUCU CGUCU CCUCG ACACC-3′. It has turned out that this nucleotide sequence is particularly effective in binding to ADAR1, in particular ADAR1p110.

In another preferred embodiment, the second recruiting moiety of the artificial nucleic acid comprises the nucleotide sequence 5′-GUG GAA UAG UAU AAC AAU AUG CUA AAU GUU GUU AUA GUA UCC CAC-3′, which in particular was shown to effectively bind to ADAR2.

Since the above sequences are adapted from a well-known ADAR2 target site in glutamate receptor 2 mRNA, they are also known as R/G motif.

Therefore, although the second recruiting moiety may be a recruiting moiety as defined with respect to the first recruiting moiety, i.e. a recruiting moiety comprising at least one recruitment sequence, and preferably comprising a cluster of recruitment sequences, which bind and preferably are complementary or at least partially complementary to a first region and preferably further regions of the target RNA, it is preferred that at least one of the first and second recruiting moiety comprises a nucleic acid sequence capable of binding to a deaminase, preferably a deaminase as defined herein.

Therefore, the artificial nucleic acid of the present invention is preferably a single-stranded (ss) nucleic acid molecule. In a preferred embodiment, the artificial nucleic acid is a single-stranded nucleic acid, which at physiological conditions comprises double-stranded (ds) regions. Preferably, the artificial nucleic acid is a single-stranded nucleic acid comprising double-stranded regions within the recruiting moiety, that is not intended to bind to the target mRNA, capable of binding a deaminase.

Accordingly, in a preferred embodiment, the artificial nucleic acid of the present invention comprises in in 5′ to 3′ direction or 3′ to 5′ direction:
a) a first recruiting moiety comprising at least one recruitment sequence which binds to a first region in the target RNA, and preferably comprising a cluster of recruitment sequences which bind to a first and further regions of the target RNA;
b) a targeting sequence which comprises a nucleic acid sequence complementary or at least partially complementary to a target sequence in the target RNA comprising one or more nucleotides to be edited, and
c) a second recruiting moiety comprising a nucleic acid sequence capable of binding a deaminase, preferably a nucleic acid sequence as defined above, for example a R/G motif.

In a more preferred embodiment, the artificial nucleic acid of the present invention comprises a) the first recruiting moiety, b) the targeting sequence, and c) the second recruiting moiety, as defined above, in 3′ to 5′ direction.

The second recruiting moiety comprising a nucleic acid sequence capable of binding a deaminase, preferably a nucleic acid sequence as defined above, for example an R/G motif, may also comprise at least one recruitment sequence which binds, and preferably is complementary or at least partially complementary, to a further region in the target RNA, or a cluster of recruitment sequences, respectively, which bind, and preferably are complementary or at least partially complementary, to further regions of the target RNA.

In some embodiments, the artificial nucleic acid of the present invention comprises in 5′ to 3′ direction or in 3′ to 5′ direction:
a) a first recruiting moiety comprising at least one recruitment sequence which binds, and preferably is complementary or at least partially complementary, to a first region in the target RNA, or comprising a cluster of recruitment sequences which bind, and preferably are complementary or at least partially complementary, to a first and further regions of the target RNA;
b) a targeting sequence which comprises a nucleic acid sequence complementary or at least partially complementary to a target sequence in the target RNA comprising one or more nucleotides to be edited, and
c) a second recruiting moiety comprising at least one recruitment sequence which binds, and preferably is complementary or at least partially complementary, to a further region in the target RNA, and a nucleic acid sequence capable of binding a deaminase, preferably a nucleic acid sequence as defined above, for example an R/G motif.

For example, the first recruiting moiety may comprise one recruitment sequence complementary or at least partially complementary to a first region in the target RNA, whereas the second recruiting moiety comprises two recruitment sequences complementary or at least partially complementary to a second and a third region in the target RNA, and a nucleic acid sequence capable of binding a deaminase. As another example, the first recruiting moiety may comprise two recruitment sequences complementary or at least partially complementary to a first and a second region in the target RNA, whereas the second recruiting moiety comprises a third recruitment sequence complementary or at least partially complementary to a third region in the target RNA, and a nucleic acid sequence capable of binding a deaminase. Preferably, the nucleic acid sequence capable of binding a deaminase, such as an R/G motif, is located at the 5′ end of the artificial nucleic acid.

To avoid off-target editing events in the double-stranded region formed by the further region(s) of the target RNA and the recruitment sequence(s), the recruitment sequence(s) of the second recruiting moiety is/are preferably depleted from uridine bases unless they are either within 5 nt from either end (5′ or 3′) of a recruitment sequence or in a 5′-NUS (S═C or G) context.

Therefore, the recruitment sequence(s) of the second recruiting moiety preferably has/have a reduced uridine content. For example, the recruitment sequence(s) of the second recruiting moiety contain(s) not more than 50%, not more than 25%, not more than 20%, not more than 15%, not more than 10%, not more than 5%, not more than 4%, not more than 3%, not more than 2%, not more than 1%, or even less than 1% uridine nucleotides with respect to the total number of nucleotides contained in the recruitment sequence(s), unless they are either within 5 nt from either end (5′ or 3′) of a recruitment sequence or in a 5′-NUS (S═C or G) context. More preferably, the recruitment sequence(s) of the second recruiting moiety of the artificial nucleic acid does/do not contain any uridine bases, with the exception of uridine bases which are either within 5 nt from either end (5′ or 3′) of a recruitment sequence or in a 5′-NUS (S═C or G) context.

In the target RNA, the first region (which is bound by the at least one recruitment sequence of the first recruiting moiety) and the target sequence (which is complementary to the targeting sequence) are separated by at least one nucleotide which is not bound by the at least one recruitment sequence and which is not complementary to the targeting sequence of the artificial nucleic acid. That is, the first region and the target sequence in the target RNA do preferably not merge into each other. The first region in the target RNA and the target sequence may preferably be separated by at least one nucleotide, preferably by 2 to 10.000 nucleotides, e.g. by 2 to 5000 nucleotides, 2 to 1000 nucleotides, 2 to 500 nucleotides, 2 to 400 nucleotides, 2 to 300 nucleotides, 2 to 200 nucleotides or 2 to 100 nucleotides. More preferably, the first region and the target sequence in the target RNA are separated by 2 to 50 nucleotides, e.g. by 2 to 40 nucleotides, 2 to 30 nucleotides, 2 to 20 nucleotides or 2 to 10 nucleotides.

Similarly, in the target RNA, the first region (which is bound by a first recruitment sequence of the first recruiting moiety) and a second or further region (which is bound by a second or further recruitment sequence of the first recruiting moiety) are preferably separated by at least one nucleotide, preferably by 2 to 10.000 nucleotides, e.g. by 2 to 5000 nucleotides, 2 to 1000 nucleotides, 2 to 500 nucleotides, 2 to 400 nucleotides, 2 to 300 nucleotides, 2 to 200 nucleotides or 2 to 100 nucleotides. More preferably, the first region and the second or further region in the target RNA are separated by 2 to 50 nucleotides, e.g. by 2 to 40 nucleotides, 2 to 30 nucleotides, 2 to 20 nucleotides or 2 to 10 nucleotides.

Accordingly, the artificial nucleic acid of the present invention which binds to the target RNA via the at least one recruitment sequence and the targeting sequence, may span a region of several 1000, 10.000 or even several 100.000 nucleotides on the target RNA thereby recruiting the deaminase to a specific target site of the target RNA. It has to be noted that when the target sequence of the target RNA bound by the targeting sequence of the artificial nucleic acid is distanced from the first region bound by the first recruitment sequence of the artificial nucleic acid by several hundred or even several thousand nucleotides, e.g. 100, 200, 300, 400, 500, 1000, 5000, 10.000 or 100.000 nucleotides, these nucleotides may form a loop in the target RNA upon binding the artificial nucleic acid.

The artificial nucleic acid according to the present invention is not limited in its length and may be, for example, an oligonucleotide. As used herein, the term ‘oligonucleotide’ may refer to short nucleic acid molecules (e.g. a 6-mer or a 10-mer) as well as to longer oligonucleotides (e.g. nucleic acid molecules comprising 100 or even 200 nucleotides), wherein the oligonucleotide may comprise (unmodified or modified) ribonucleotides and/or (unmodified or modified) deoxynucleotides. According to a preferred embodiment, the artificial nucleic acid comprises at least about 15, preferably at least about 20, more preferably at least about 25, even more preferably at least about 30, even more preferably at least about 35, most preferably at least about 40, nucleotides. Alternatively, the length of the artificial nucleic acid is in the range from about 15 to about 1000 nucleotides, e.g. from about 15 to about 400 nucleotides, from about 15 to about 300 nucleotides, from about 15 to about 200 nucleotides, preferably from about 20 to about 150 nucleotides, more preferably from about 20 to about 100 nucleotides, most preferably from about 20 to about 80 nucleotides.

In particular embodiments of the present invention, the artificial nucleic acid may comprise nucleotides which are chemically modified. As used herein, the term ‘chemical modification’ preferably refers to a chemical modification selected from backbone modifications, sugar modifications or base modifications, including abasic sites. A ‘chemically modified nucleic acid’ in the context of the present invention may refer to a nucleic acid comprising at least one chemically modified nucleotide.

In particular embodiments of the present invention, the first recruiting moiety and/or the targeting sequence and/or the second recruiting moiety of the artificial nucleic acid may comprise at least one chemically modified nucleotide. Particularly, the first recruiting moiety and/or the targeting sequence and/or the second recruiting moiety may comprise a plurality of chemically modified nucleotides, which may result in specific modification patterns which are e.g. disclosed in WO/2020/001793.

Generally, the artificial nucleic acid molecule of the present invention may comprise native (=naturally occurring) nucleotides as well as chemically modified nucleotides. As used herein, the term ‘nucleotide’ generally comprises (unmodified and modified) ribonucleotides as well as (unmodified and modified) deoxynucleotides. The term ‘nucleotide’ thus preferably refers to adenosine, deoxyadenosine, guanosine, deoxyguanosine, inosine, deoxyinosine, 5-methoxyuridine, thymidine, uridine, deoxyuridine, cytidine, deoxycytidine or to a variant thereof. Moreover, where reference is made herein to a‘nucleotide’, the respective nucleoside is preferably comprised as well.

In this respect, a ‘variant’ of a nucleotide is typically a naturally occurring or an artificial variant of a nucleotide. Accordingly, variants are preferably chemically derivatized nucleotides with non-natively occurring functional groups, which are preferably added to or deleted from the naturally occurring nucleotide or which substitute the naturally occurring functional groups of a nucleotide. Accordingly, in such a nucleotide variant each component of the naturally occurring nucleotide, preferably a ribonucleotide or a deoxynucleotide, may be modified, namely the base component, the sugar (ribose) component and/or the phosphate component forming the backbone of the artificial nucleic acid, preferably by a modification as described herein. The term ‘variant (of a nucleotide, ribonucleotide, deoxynucleotide, etc.)’ thus also comprises a chemically modified nucleotide, preferably as described herein.

A chemically modified nucleotide as used herein is preferably a variant of guanosine, uridine, adenosine, thymidine and cytidine including, without implying any limitation, any natively occurring or non-natively occurring guanosine, uridine, adenosine, thymidine or cytidine that has been altered chemically, for example by acetylation, methylation, hydroxylation, etc., including 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2′-O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytodine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. The preparation of such variants is known to the person skilled in the art, for example from U.S. Pat. Nos. 4,373,071, 4,401,796, 4,415,732, 4,458,066, 4,500,707, 4,668,777, 4,973,679, 5,047,524, 5,132,418, 5,153,319, 5,262,530 or 5,700,642.

In some embodiments, the artificial nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, puromycin-5′-triphosphate, or xanthosine-5′-triphosphate.

In some embodiments, the artificial nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, the artificial nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, the artificial nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In certain embodiments, the artificial nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

In certain embodiments, the artificial nucleic acid comprises at least one chemically modified nucleotide, which is chemically modified at the 2′ position. Preferably, the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. In the context of the artificial nucleic acid, in particular if the artificial nucleic acid is an RNA or a molecule comprising ribonucleotides, a 2′-deoxynucleotide (comprising hydrogen as a substituent at the 2′ carbon atom), such as deoxycytidine or a variant thereof, may also be referred to as ‘chemically modified nucleotide’.

Another chemical modification that involves the 2′ position of a nucleotide as described herein is a locked nucleic acid (LNA) nucleotide, an ethylene bridged nucleic acid (ENA) nucleotide and an (S)-constrained ethyl cEt nucleotide. These backbone modifications lock the sugar of the modified nucleotide into the preferred northern conformation. It is believed that the presence of that type of modification in the targeting sequence of the artificial nculeic acid allows for stronger and faster binding of the targeting sequence to the target RNA.

According to some embodiments, the artificial nucleic acid comprises at least one chemically modified nucleotide, wherein the phosphate backbone, which is incorporated into the artificial nucleic acid molecule, is modified. The phosphate groups of the backbone can be modified, for example, by replacing one or more of the oxygen atoms with a different substituent. Further, the modified nucleotide can include the full replacement of an unmodified phosphate moiety with a modified phosphate as described herein. Examples of modified phosphate groups include, but are not limited to, the group consisting of a phosphorothioate, a stereopure phosphorothioate, a phosphoroselenate, a borano phosphate, a borano phosphate ester, a hydrogen phosphonate, a phosphoroamidate, an alkyl phosphonate, an aryl phosphonate and a phosphotriester. The phosphate linker can also be modified by the replacement of a linking oxygen with nitrogen (bridged phosphoroamidates), sulfur (bridged phosphorothioates) and carbon (bridged methylene-phosphonates).

According to a further preferred embodiment, the artificial nucleic acid comprises an abasic site. As used herein, an ‘abasic site’ is a nucleotide lacking the organic base. In preferred embodiments, the abasic nucleotide further comprises a chemical modification as described herein at the 2′ position of the ribose. Preferably, the 2′ C atom of the ribose is substituted with a substituent selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. In the context of the present invention, a ‘chemically modified nucleotide’ may therefore also be an abasic site.

According to another embodiment, the artificial nucleic acid molecule can be modified by the addition of a so-called ‘5′ CAP’ structure. A 5′-cap is an entity, typically a modified nucleotide entity, which generally ‘caps’ the 5′-end of a mature mRNA. A 5′-cap may typically be formed by a modified nucleotide, particularly by a derivative of a guanine nucleotide. Preferably, the 5′-cap is linked to the 5′-terminus of the artificial nucleic acid via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. Particularly preferred modified 5′-CAP structures are CAP1 (methylation of the ribose of the adjacent nucleotide of m7G), CAP2 (methylation of the ribose of the 2nd nucleotide downstream of the m7G), CAP3 (methylation of the ribose of the 3rd nucleotide downstream of the m7G), CAP4 (methylation of the ribose of the 4th nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the artificial nucleic acid comprises a moiety, which enhances cellular uptake of the artificial nucleic acid. Preferably, the moiety enhancing cellular uptake is a triantennary N-acetyl galactosamine (GalNAc3), which is preferably conjugated with the 3′ terminus or with the 5′ terminus of the artificial nucleic acid.

In a preferred embodiment of the present invention, the artificial nucleic acid comprises unmodified ribonucleotides and/or unmodified deoxynucleotides. More preferably, the artificial nucleic acid according to the present invention is an RNA or RNA analog. Most preferably, the artificial nucleic acid according to the present invention is an endogenously expressible RNA.

In one aspect, the present invention provides a method for generating the sequence of an artificial nucleic acid for site-directed editing of a target RNA as defined above.

The method for generating the sequence of an artificial nucleic acid for site-directed editing of a target RNA comprises the steps of
i) generating a sequence of a first recruiting moiety, wherein the first recruiting moiety comprises at least one recruitment sequence which binds to a first region in the target RNA;
ii) optionally generating a sequence of a nucleotide spacer comprising at least 1 nucleotide;
iii) generating a targeting sequence which comprises a nucleic acid sequence complementary to or at least partially complementary to a target sequence in the target RNA comprising one or more nucleotides to be edited;
iv) generating a sequence of a second recruiting moiety capable of recruiting a deaminase; and
v) assembling the sequence of the first recruiting moiety, optionally the nucleotide spacer, the targeting sequence and the sequence of the second recruiting moiety in 5′ to 3′ direction or 3′ to 5′ direction.

In a preferred embodiment, in the method for generating the sequence of an artificial nucleic acid, step i) and optionally step iv) comprises generating a recruiting moiety comprising at least two recruitment sequences, preferably generating a cluster of recruitment sequences, which are linked via a nucleotide linker.

The generated nucleic acid sequence may be expressed in vitro or in vivo thereby synthesizing the artificial nucleic acid molecule of the present invention. The artificial nucleic acid synthesized on the basis of the generated sequence is suitable for editing an RNA target with high efficiency and high specificity, in particular with a reduced rate of off-target editing, as described above.

The first recruiting moiety, and optionally also the second recruiting moiety of the artificial nucleic acid of the present invention comprises at least one recruitment sequence, and preferably a cluster of recruitment sequences, which bind to a first and optionally further regions of the target RNA. To avoid undesirable off-targeting events in the double-stranded region formed by the at least one recruitment sequence and the first and further regions of the target RNA, the target RNA which is bound by the at least one recruitment sequence preferably does not comprise any editable nucleotides, and more preferably does not comprise any editable adenosine nucleotide(s). Therefore, to generate (a) recruitment sequence(s) of the first and optionally second recruiting moiety of the artificial nucleic acid which bind(s) to a first and optionally further region(s) of the target RNA, the target RNA is preferably screened for regions which are suitable as binding site for the at least one recruitment sequence of the artificial nucleic acid and which preferably do not comprise any editable adenosine nucleotide(s). Since selection of the first and further regions “by hand” may be very laborious, said screening step is preferably accomplished by means of a computer program (“Recruitment Cluster Finder” (RCF)), which will be explained later.

Consequently, in the method for generating the sequence of an artificial nucleic acid of the present invention, step i), and optionally step iv), is/are preferably accomplished by a computer implemented method comprising the step of screening the target RNA to be edited for a nucleic acid sequence which is suitable as binding site for at least one recruitment sequence and which preferably does not comprise any editable adenosine nucleotide(s).

When using a computer implemented method for generating the sequence of a first and optionally a second recruiting moiety of an artificial nucleic acid of the present invention, further specification(s), such as the length of nucleic acid sequences suitable as binding sites for recruitment sequences on the target RNA, and the distance between binding sites for individual recruitment sequences on the target RNA, may be made to generate a beneficial or even optimum sequence of the recruiting sequence(s).

In a more preferred embodiment of the method for generating the sequence of an artificial nucleic acid of the present invention, all steps (i) to (v) are computer implemented.

Preferably, in the computer implemented method of the present invention, the sequence of an inventive artificial nucleic acid is e.g. generated based on presets in terms of
the length of the nucleic acid sequence serving as a binding site for the targeting sequence on the target RNA,
the distance between the binding site for the targeting sequence and the binding site for the at least one recruitment sequence on the target RNA,
the length of the nucleotide linker and, optional, the nucleotide spacer,
the length of nucleic acid sequences suitable as binding sites for recruitment sequences on the target RNA, and/or
the distance between binding sites for individual recruitment sequences on the target RNA.

As part of the presets used in the computer implemented method of the present invention, the length of the nucleic acid sequence serving as a binding site for the targeting sequence on the target RNA may be set e.g. in a range of 5 to 100 nucleotides, e.g. 5 to 90, 5 to 80, 5 to 70, 5 to 60, 5 to 50, 5 to 40 nucleotides, and is preferably set in a range of 10 to 50, more preferably 16 to 40 nucleotides.

Furthermore, the distance between the binding site for the targeting sequence (target sequence) and the binding site for the at least one recruitment sequence (first region) in the target RNA is set to at least 1 nucleotide. Preferably, the distance between the target sequence and the first region in the target RNA is set in a range from 2 to 100.000 nucleotides, for example 2 to 100.000, 2 to 10.000, 2 to 5000, 2 to 1000, 2 to 500, 2 to 400, 2 to 300, 2 to 200, 2 to 100 nucleotides, and more preferably is set in a range from 2 to 50 nucleotides.

Moreover, the length of the nucleotide linker linking the recruitment sequences of a recruitment cluster and optionally the nucleotide spacer located between the first recruiting moiety and the targeting sequence of the artificial nucleic acid is set to at least 1 nucleotide. Preferably, the length of the nucleotide linker and optionally the nucleotide spacer is set in a range from 1 to 100 nucleotides, e.g. 1 to 90 nucleotides, 1 to 80 nucleotides, 1 to 70 nucleotides, 1 to 60 nucleotides, 1 to 50 nucleotides, 1 to 40 nucleotides, 1 to 30 nucleotides, 1 to 20 nucleotides, 1 to 10 nucleotides, and preferably is set in a range from 2 to 6 nucleotides.

As part of the presets used in the computer implemented method, the length of nucleic acid sequences suitable as binding sites for recruitment sequences on the target RNA may be set in a range of at least 10 nucleotides, e.g. 10 to 500, 10 to 400, 10 to 300, or 10 to 200 nucleotides, or at least 20 nucleotides, preferably 20 to 100 nucleotides.

Further, in the computer implemented method for generating the sequence of an artificial nucleic acid according to the present invention, the distance between individual binding sites for recruitment sequences on the target RNA is set to at least 1 nucleotide, and e.g. is set in a range of 1 to 100.000 nucleotides, e.g. 1 to 100.000, 1 to 10.000, 1 to 5000, 1 to 1000, 1 to 900, 1 to 800, 1 to 700, 1 to 600, 1 to 500, 1 to 400, 1 to 300, 1 to 200 or 1 to 100 nucleotides, and preferably is set in a range of 2 to 500 nucleotides, e.g. 2 to 400, 2 to 300, 2 to 200, 2 to 100 nucleotides, and is more preferably set in a range of 2 to 50 nucleotides.

By means of the above presets, at least one nucleotide sequence is created by the computer implemented method on the basis of which the artificial nucleic acid of the present invention may be synthesized. In a preferred embodiment, the computer implemented method for generating the sequence of an artificial nucleic acid of the present invention comprises creating alternative sequences of the artificial nucleic acid, in particular alternative sequences of a first recruiting moiety comprising a cluster of recruitment sequences.

In a more preferred embodiment, the computer implemented method for generating the sequence of an artificial nucleic acid of the present invention further comprises the step of determining potential intramolecular base pairing events within the assembled artificial nucleic acid. In this way, out of the created alternative sequences of a potential artificial nucleic acid, those artificial nucleic acids may be detected which present a minimal potential of intramolecular base pairing and may be selected as artificial nucleic acid molecules which are particularly suitable for site-directed editing of a particular target RNA.

To this end, the computer implemented method may further comprise a step of evaluating whether the selected recruitment sequence or the selected cluster of recruitment sequences, preferably the artificial nucleic acid, is able to bind to other sites on the target RNA or to another RNA molecule in the transcriptome of an organism, resulting in unwanted mismatches and off-targeting events, and excluding such a recruitment sequence or such a cluster of recruitment sequences.

In certain embodiments of the present invention, step iv) of the above method for generating the sequence of an artificial nucleic acid comprises the same steps as defined with respect to step i). That is, as the sequence of the first recruiting moiety, the sequence of the second recruiting moiety is generated as a sequence comprising at least one recruitment sequence, and preferably comprising a cluster of recruitment sequences which bind to specific regions on the target RNA. In other words, the artificial nucleic acid generated by methods of the present invention may comprise at least one recruitment sequence, and preferably a cluster of recruitment sequences 3′ and 5′ of the targeting sequence.

However, as mentioned above, in preferred embodiments, the second recruiting moiety of the artificial nucleic acid comprises a nucleic acid sequence capable of binding to a deaminase. Therefore, in preferred embodiments, a nucleic acid sequence capable of binding to a deaminase, preferably a nucleic acid sequence as defined above, is preset as second recruiting moiety in the method for generating the sequence of an artificial nucleic acid of the present invention.

As mentioned above, the first recruiting moiety, optionally the nucleotide spacer, the targeting sequence and the second recruiting moiety may be assembled in 5′ to 3′ direction or 3′ to 5′ direction. Preferably, the sequences of the components of the artificial nucleic acid are assembled in 3′ to 5′ direction.

In preferred embodiments of the present invention, all steps i) to v) described above and also further steps of determining potential intramolecular base pairing events and of evaluating whether the artificial nucleic acid is able to bind to other sites on the target RNA or to another molecule in the transcriptome of a specific organism, are computer implemented thereby creating the optimal sequence of an artificial nucleic acid for site-directed editing of a specific target RNA.

Consequently, a preferred method for generating the sequence of an artificial nucleic acid for site-directed editing of a target RNA comprises the steps of
i) generating a sequence of a first recruiting moiety comprising at least one recruitment sequence, and preferably comprising a cluster of at least three, preferably 3 to 10, recruitment sequences each comprising at least 10, preferably 15 to 100 nucleotides, which bind to a first and further regions in the target RNA, and which are linked via a nucleotide linker, more preferably an adenosine linker;
ii) generating a sequence of a nucleotide spacer comprising at least 1 nucleotide, preferably 2 to 6 nucleotides, more preferably adenosine nucleotides;
iii) generating a targeting sequence which comprises a nucleic acid sequence complementary or at least partially complementary to a target sequence in the target RNA comprising one or more nucleotides to be edited and comprising at least 10, preferably 16 to 40 nucleotides;
iv) generating a sequence of a second recruiting moiety comprising a nucleic acid sequence capable of binding to a deaminase, preferably an adenosine deaminase, without binding to the target RNA, preferably a sequence as defined above;
v) assembling the sequence of the first recruiting moiety, the nucleotide spacer, the targeting sequence and the sequence of the second recruiting moiety in 5′ to 3′ direction or 3′ to 5′ direction, more preferably in 3′ to 5′ direction;
vi) determining potential intramolecular base pairing events within the assembled artificial nucleic acid and selecting an artificial nucleic acid presenting a minimal potential of intramolecular base pairing;
vii) evaluating whether the selected recruitment sequence or the selected cluster of recruitment sequences, preferably the artificial nucleic acid, is able to bind to other sites on the target RNA or to another RNA molecule in the transcriptome of an organism, resulting in unwanted mismatches and off-targeting events, and excluding such a recruitment sequence or such a cluster of recruitment sequences.

The artificial nucleic acid as described herein may be synthesized on the basis of the sequence created by the inventive method by a method known in the art. The artificial nucleic acid may be synthesized chemically or by in vitro transcription from a suitable vector, preferably as described herein. Preferably, the artificial nucleic acid of the present invention is synthesized in vivo from a suitable vector, as described herein, which has previously been transfected in a cell or organism.

In a further aspect, the present invention is directed to a data processing device comprising means configured to perform the method for generating the sequence of an artificial nucleic acid for site-directed editing of a target RNA by
i) generating a sequence of a first recruiting moiety, wherein the first recruiting moiety comprises at least one recruitment sequence, which binds to a first region in the target RNA;
ii) optionally generating a sequence of a nucleotide spacer comprising at least 1 nucleotide;
iii) generating a targeting sequence which comprises a nucleic acid sequence complementary or at least partially complementary to a target sequence in the target RNA comprising one or more nucleotides to be edited;
iv) generating a sequence of a second recruiting moiety capable of recruiting a deaminase; and
v) assembling the sequence of the first recruiting moiety, optionally the nucleotide spacer, the targeting sequence and the sequence of the second recruiting moiety in 5′ to 3′ direction or 3′ to 5′ direction.

Preferably, the data processing device is configured to perform the method of the present invention based on the sequence information of the RNA to be edited and on presets input by a user in terms of
the length of the nucleic acid sequence serving as a binding site for the targeting sequence on the target RNA,
the distance between the binding site for the targeting sequence and the binding site for the at least one recruitment sequence on the target RNA,
the length of the nucleotide linker and, optional, the nucleotide spacer,
the length of nucleic acid sequences suitable as binding sites for recruitment sequences on the target RNA, and/or
the distance between binding sites for individual recruitment sequences on the target RNA, a candidate for an artificial nucleic acid for site-directed editing of a target RNA according to the present invention.

In a more preferred embodiment, the data processing device is configured to
vi) determine potential intramolecular base pairing events within the assembled artificial nucleic acid and select an artificial nucleic acid presenting a minimal potential of intramolecular base pairing, and/or
vii) evaluate whether the selected recruitment sequence or the selected cluster of recruitment sequences, preferably the artificial nucleic acid, is able to bind to other sites on the target RNA or to another RNA molecule in the transcriptome of an organism, resulting in unwanted mismatches and off-targeting events, and excluding such a recruitment sequence or such a cluster of recruitment sequences.

In a further aspect, the present invention is directed to a computer program comprising instructions which, when the program is executed by a computer, cause the computer to carry out the method for generating the sequence of an artificial nucleic acid for site-directed editing of a target RNA based on the sequence information of the RNA to be edited and on the presets input by a user described above.

As a basic function, the computer program includes a ‘Recruitment Cluster Finder’ which generates the sequence of a recruitment cluster comprising a number of recruitment sequences constituting the recruiting moiety of the artificial nucleic acid.

To this end, the ‘Recruitment Cluster Finder’ may include following key functions:
Function 1: This function searches for G, C, T, GA blocks within the input target sequence.

```
def findRangelndices( ):
  blockMin = vBlockSizeMin.get( )
  blockMax = vBlockSizeMax.get( )
  hits = vORF.get( )
  hitsSplitted = hits.split(vGuide1.get( ))
  hitsReplaced = re.sub(r‘GA’, ‘bb’, hitsSplitted[0], flags=re.I)
  hitsReplaced = re.sub(r‘[GTC]’, ‘B’, hitsReplaced, flags=re.I)
  my_Regex = r“B{“ + re.escape(blockMin) + r”,}”
  result = re.finditer(my_Regex, hitsReplaced, flags=re.I)
  resultList = [ ]
  for x in result:
    resultList.insert(0, (x.start(0), x.end(0))) # for reverse list
  print(“\nPossible Block Range Indices of ” + str(len(resultList)) + “ Blocks: \n”)
  guideIndex = len(hitsSplitted[0])
  print(“Guide Sequence at “ + str(guideIndex) + ”, Distance to next Block: ” + str(guideIndex −
resultList[0][1]))
  print(resultListToString(resultList))
  return (hits, resultList, guideIndex)
```

Function 2: This function recombines the recruitment sequences to a recruitment cluster. The recruitment clusters are then filtered for the input criteria, recruitment sequence sizes and distances. All groups that fulfil the criteria (hits) are saved into a file that is later used by ViennaRNA.

```
def recombineAllLists(lis):
  result = None
  for curr in lis:
    if (result == None):
      result = [[x] for x in curr]
    else:
      temp = [ ]
      for x in range(0, len(result)):
        temp = temp + [result[x] + [y] for y in curr]
      result = temp
  return (result)
for currBlock in allBlocksAll:
          bool = True
          bool = (distToGuideSeq − currBlock[clusterNumber-1][1])>=
          distanceMin and (distToGuideSeq − currBlock[clusterNumber-1] [1
          ]) <= distanceMax
          for i in sizeRange:
            if(not bool):
              break
            bool = bool and (currBlock[i+1] [0] − currBlock[i] [1]) >=
            distanceMax and (currBlock[i+1] [0]−currBlock[i] [1] <=
            distanceMax
          if (bool):
            counterDistanceGood += 1
            clustStr = “> ”
            for n in currBlock:
              clustStr = clustStr + tupleToStr(n)
            file.write(clustStr + “\n” + generateRcRNA(currBlock) +
            “\n”)
          else:
            counterDistanceBad += 1
  print(“Hits:” + str(counterDistanceGood) + “ Filter out: ” + str(
  counterDistanceBad))
```

Function 3: This function converts blocks complementary.

```
def generateRcRNA(blocks):
  dna = vOptional.get( )
  for block in blocks:
    dna = dna + block[2] + vLinker.get( )
  dna = dna + vGuide2.get( ) + vMotif.get( )
  rcRNA = Seq(dna).transcribe( ).reverse_complement( )
  return (str(rcRNA))
```

Function 4: This function runs ViennaRNA [196] to fold the guideRNAs. The results are saved in an output list.

```
def calcOutput( ):
  startTime = time.time( )
  output = subprocess.check_output([“ViennaRNA Package/RNAfold.exe”, “-- noPS”,
“temp/sequences.txt”])
  root.outputList = outputToList(output)
  printCalculatedOutput( )
  print(“Run Time: ” + str(time.time( ) − startTime))
  print(“\nCalculation completed!”)
```

Function 5: The ViennaRNA results are sorted by the initially chosen criteria (“numerical order”, “dot/bracket ratio order” or “minimal energy order”). Then a result table is generated.

```
def printCalculatedOutput( ):
  outputList = root.outputList
  if (len(outputList) > 0):
    max = min(int(vNumberResults.get( )), (len(outputList[O])))
    order = vOutputSort.get( )
    if (order == sMinEnergy):
      bestSortedValues = heapq.nsmallest(max, outputList[4])
      bestSortedValues = list(sorted(set(bestSortedValues), reverse=False))
    else:
      if (order == sDotBracket):
        bestSortedValues = heapq.nlargest(max, outputList[5])
        bestSortedValues = list(sorted(set(bestSortedValues), reverse =
        True))
      else:
        bestSortedValues = outputList [5][:max]
    isShowBlocks = (vShowBlocks.get( ) == 1)
    listbox.delete(0, Tk.END)
    result = [ ]
    for current in bestSortedValues:
      if (order == sMinEnergy):
        indices = [i for i, x in enumerate(outputList[4]) if x == current]
      else:
        if (order == sDotBracket):
          indices = [i for i, x in enumerate(outputList[5]) if x ==
          current]
        else:
          indices = range(0, max)
      for currentMin in indices:
        if (max > 0):
          string1 = str(outputList[1 ][currentMin]) + “\n”
          string2 = str(outputList[2][currentMin]) + “\n”
          string3 = str(outputList[3][currentMin]) + “\n”
          if (isShowBlocks):
            seq = outputList[1][currentMin]
            blockIndices = [int(match.group( )) for match in re.finditer
            (“[0-9]+”, seq)]
            orf = vORF.get( )
            stringSeq1 = orf[blockIndices[0]: blockIndices[1]] + “\n”
            stringSeq2 = orf[blockIndices[2]: blockIndices[3]] + “\n”
            stringSeq3 = orf[blockIndices[4]: blockIndices[5] + “\n”
          string4 = “ Energy: “ + str(outputList[4] [currentMin]) + ”,
          Dots/Brackets Ratio: “ + str(outputList[5][currentMin]) + ”\n”
          listbox.insert(Tk.END, string1)
          listbox.insert(Tk.END, string2)
          listbox.insert(Tk.END, string3)
          if (isShowBlocks):
            listbox.insert(Tk.END, stringSeq1)
            listbox.insert(Tk.END, stringSeq2)
            listbox.insert(Tk.END, stringSeq3)
          listbox.insert(Tk.END, string4)
          if (isShowBlocks):
            result.append([string1, string2, string3, stringSeq1,
            stringSeq2, stringSeq3, string4])
          else:
            result.append([string1, string2, string3, string4])
          max −= 1
    root.exportResult = result
```

Furthermore, the present invention is directed to a computer-readable storage medium comprising instructions which, when executed by a computer, cause the computer to carry out the method for generating the sequence of an artificial nucleic acid for site-directed editing of a target RNA. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. A specific example of a computer-readable portable memory device is an USB flash memory device. A computer readable storage medium may be any tangible medium that can contain, or store a program for use by or in connection with an instruction execution system, apparatus, or device.

In one aspect, the present invention provides a vector encoding the artificial nucleic acid described herein.

The term ‘vector’ as used herein typically refers to a nucleic acid molecule, preferably to an artificial nucleic acid molecule. A vector in the context of the present invention is suitable for incorporating or harbouring a desired nucleic acid sequence, such as the nucleic acid sequence of the artificial nucleic acid or a fragment thereof. Such vectors may be storage vectors, expression vectors, cloning vectors, transfer vectors etc. A cloning vector may be, e.g., a plasmid vector or a bacteriophage vector. A transfer vector may be a vector, which is suitable for transferring nucleic acid molecules into cells or organisms, for example, viral vectors. Preferably, a vector in the sense of the present application comprises a cloning site, a selection marker, such as an antibiotic resistance factor, and a sequence suitable for multiplication of the vector, such as an origin of replication.

The vector may be an RNA vector or a DNA vector. Preferably, the vector is a DNA vector. The vector may be any vector known to the skilled person, such as a viral vector or a plasmid vector. Preferably, the vector is a plasmid vector, preferably a DNA plasmid vector. In certain embodiments, the vector is a viral vector, which is preferably selected from the group consisting of lentiviral vectors, retroviral vectors, adenoviral vectors, adeno-associated viral (AAV) vectors and hybrid vectors.

Preferably, the vector according to the present invention is suitable for producing the artificial nucleic acid molecule, preferably an RNA, according to the present invention. Thus, preferably, the vector comprises elements needed for transcription, such as a promoter, e.g. an RNA polymerase promoter. Preferably, the vector is suitable for transcription using eukaryotic, prokaryotic, viral or phage transcription systems, such as eukaryotic cells, prokaryotic cells, or eukaryotic, prokaryotic, viral or phage in vitro transcription systems. Thus, for example, the vector may comprise a promoter sequence, which is recognized by a polymerase, such as by an RNA polymerase, e.g. by a eukaryotic, prokaryotic, viral, or phage RNA polymerase. In a preferred embodiment, the vector comprises a phage RNA polymerase promoter such as an SP6, T3 or T7, preferably a T7 promoter. Preferably, the vector is suitable for in vitro transcription using a phage based in vitro transcription system, such as a T7 RNA polymerase based in vitro transcription system.

In some embodiments, the vector is designed for transcription of the artificial nucleic acid upon transfection into a eukaryotic cell, preferably upon transfection into a mammalian cell, or upon administration to a subject, preferably as described herein. In a preferred embodiment, the vector is designed for transcription of the artificial nucleic acid by a eukaryotic RNA polymerase, preferably RNA polymerase II or III, more preferably RNA polymerase III. In certain embodiments, the vector may comprise a U6 snRNA promoter or a H1 promoter and, optionally, a selection marker, e.g. a reporter gene (such as GFP) or a resistance gene (such as a puromycin or a hygromycin resistance gene).

According to one aspect of the present invention, a cell is provided that comprises the artificial nucleic acid or the vector described herein. The cell may be any cell, such as a bacterial cell or a eukaryotic cell, preferably an insect cell, a plant cell, a vertebrate cell, such as a mammalian cell (e.g. a human cell or a murine cell). The cell may be, for example, used for replication of the vector of the present invention, for example, in a bacterial cell. Furthermore, the cell, preferably a eukaryotic cell, may be used for synthesis of the artificial nucleic acid molecule according to the present invention.

The cells according to the present invention are, for example, obtainable by standard nucleic acid transfer methods, such as standard transfection, transduction or transformation methods. The term ‘transfection’ as used herein generally refers to the introduction of nucleic acid molecules, such as DNA or RNA (e.g. mRNA) molecules, into cells, preferably into eukaryotic cells. In the context of the present invention, the term ‘transfection’ encompasses any method known to the skilled person for introducing nucleic acid molecules into cells, preferably into eukaryotic cells, e.g. into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In this context, the artificial nucleic acid or the vector as described herein may be introduced into the cell in a transient approach or in order to maintain the artificial nucleic acid or the vector stably in the cell (e.g. in a stable cell line).

Preferably, the cell is a mammalian cell, such as a cell of human subject, a domestic animal, a laboratory animal, such as a mouse or rat cell. Preferably, the cell is a human cell. The cell may be a cell of an established cell line, such as a CHO, BHK, 293T, COS-7, HELA, HEK, Jurkat cell line etc., or the cell may be a primary cell, such as a human dermal fibroblast (HDF) cell etc., preferably a cell isolated from an organism. In a preferred embodiment, the cell is an isolated cell of a mammalian subject, preferably of a human subject.

In a further aspect, the present invention concerns a composition comprising the artificial nucleic acid, the vector or the cell as described herein and, optionally, an additional excipient, preferably a pharmaceutically acceptable excipient. The composition described herein is preferably a pharmaceutical composition. The composition described herein may be used in treatment or prophylaxis of a subject, such as in a gene therapy approach. Alternatively, the composition can also be used for diagnostic purposes or for laboratory use, e.g. in in vitro experiments.

Preferably, the composition further comprises one or more vehicles, diluents and/or excipients, which are preferably pharmaceutically acceptable. In the context of the present invention, a pharmaceutically acceptable vehicle typically includes a liquid or non-liquid basis for the composition described herein. In one embodiment, the composition is provided in liquid form. In this context, preferably, the vehicle is based on water, such as pyrogen-free water, isotonic saline or buffered (aqueous) solutions, e.g. phosphate, citrate etc. buffered solutions. The buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of mammalian cells due to osmosis or other concentration effects. Reference media are, for instance, liquids occurring in in vivo methods, such as blood, lymph, cytosolic liquids, or other body liquids, or e.g. liquids, which may be used as reference media in in vitro methods, such as common buffers or liquids. Such common buffers or liquids are known to a skilled person. Ringer-Lactate solution is particularly preferred as a liquid basis.

One or more compatible solid or liquid fillers or diluents or encapsulating compounds suitable for administration to a subject may be used as well for the inventive pharmaceutical composition. The term “compatible” as used herein preferably means that these components of the (pharmaceutical) composition are capable of being mixed with the artificial nucleic acid, the vector or the cells as defined herein in such a manner that no interaction occurs which would substantially reduce the pharmaceutical effectiveness of the composition under typical use conditions.

The composition according to the present invention may optionally further comprise one or more additional pharmaceutically active components. A pharmaceutically active component in this context is a compound that exhibits a therapeutic effect to heal, ameliorate or prevent a particular indication or disease. Such compounds include, without implying any limitation, peptides or proteins, nucleic acids, (therapeutically active) low molecular weight organic or inorganic compounds (molecular weight less than 5000, preferably less than 1000), sugars, antigens or antibodies, or other therapeutic agents already known in the prior art.

Furthermore, the composition may comprise a carrier for the artificial nucleic acid molecule or the vector. Such a carrier may be suitable for mediating dissolution in physiological acceptable liquids, transport and cellular uptake of the pharmaceutical active artificial nucleic acid molecule or the vector. Accordingly, such a carrier may be a component, which is suitable for depot and delivery of an artificial nucleic acid molecule or vector described herein. Such components may be, for example, cationic or polycationic carriers or compounds, which may serve as transfection or complexation agent. Particularly preferred transfection or complexation agents, in this context, are cationic or polycationic compounds,

The term ‘cationic compound’ typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4. Accordingly, a cationic compound may be any positively charged compound or polymer, preferably selected from a cationic peptide or protein or a cationic lipid, which is positively charged under physiological conditions, particularly under physiological conditions in vivo. A ‘cationic peptide or protein’ may contain at least one positively charged amino acid, or more than one positively charged amino acid, e.g. selected from Arg, His, Lys or Orn. Accordingly, ‘polycationic compounds’ are also within the scope exhibiting more than one positive charge under the conditions given.

The composition as described herein preferably comprises the artificial nucleic acid or the vector in naked form or in a complexed form. In a preferred embodiment, the composition comprises the artificial nucleic acid or the vector in the form of a nanoparticle, preferably a lipid nanoparticle or a liposome.

According to a further aspect, the invention relates to a kit or kit of parts comprising the artificial nucleic acid molecule, the vector, the cell, and/or the (pharmaceutical) composition according to the invention.

Preferably, the kit additionally comprises instructions for use, cells for transfection, means for administration of the composition, a (pharmaceutically acceptable) carrier or vehicle and/or a (pharmaceutically acceptable) solution for dissolution or dilution of the artificial nucleic acid molecule, the vector, the cells or the composition. In preferred embodiments, the kit comprises the artificial nucleic acid or the vector described herein, either in liquid or in solid form (e.g. lyophilized), and a (pharmaceutically acceptable) vehicle for administration. For example, the kit may comprise the artificial nucleic acid or the vector and a vehicle (e.g. water, PBS, Ringer-Lactate or another suitable buffer), which are mixed prior to administration to a subject.

In a further aspect, the present invention concerns the use of the artificial nucleic acid, the vector, the cell, the composition or the kit described herein.

In particular, the invention comprises the use of the artificial nucleic acid, the vector, the cell, the composition or the kit for site-directed editing of a target RNA. Therein, the artificial nucleic acid, the vector, the cell, the composition or the kit described herein is preferably used to promote site-specific editing of a target RNA, preferably by specifically binding to the target RNA via the targeting sequence and by at least one recruitment sequence, thereby recruiting to the target site a deaminase as described herein. That reaction may take place in vitro or in vivo.

In a preferred embodiment, the artificial nucleic acid, the vector or the composition is administered or introduced into a cell comprising a target RNA to be edited. Said cell comprising a target RNA preferably further comprises a deaminase, preferably as described herein. Said deaminase is preferably an endogenous deaminase, more preferably an adenosine or a cytidine deaminase, or a recombinant deaminase (such as a tagged deaminase or a mutant deaminase, preferably as described herein), which is either stably expressed in said cell or introduced into said cell, preferably prior or concomitantly with the artificial nucleic acid, the vector or the composition. Alternatively, the cell comprising the artificial nucleic acid or the vector described herein is used for site-directed editing of a target RNA by bringing into contact the cell and the target RNA or by introducing the target RNA into the cell, e.g. by transfection, preferably as described herein.

In a further preferred embodiment, the invention provides a method for site-directed editing of a target RNA, which comprises contacting a target RNA with the artificial nucleic acid and which essentially comprises the steps as described herein with respect to the use of the artificial nucleic acid, the vector, the composition or the cell for site-directed editing of an RNA.

The editing reaction is preferably monitored or controlled by sequence analysis of the target RNA.

The use and the method described herein may further be employed for in vitro diagnosis of a disease or disorder. Therein, the disease or disorder is preferably selected from the group consisting of infectious diseases, tumour diseases, cardiovascular diseases, autoimmune diseases, allergies and neurological diseases or disorders, and is more preferably selected from genetic diseases or genetic disorders, which are preferably selected from the group consisting of metabolic diseases, tumour diseases, autoimmune diseases, cardiovascular diseases and neurological diseases.

In a further aspect, the artificial nucleic acid, the vector, the cell, the composition, or the kit described herein is provided for use as a medicament, e.g. in gene therapy. Preferably, the artificial nucleic acid, the vector, the composition, the cell or the kit described herein is provided for use in the treatment or prophylaxis of a disease or disorder selected from the group consisting of infectious diseases, tumour diseases, cardiovascular diseases, autoimmune diseases, allergies and neurological diseases or disorders. According to a preferred embodiment, the artificial nucleic acid, the vector, the cell, the composition, or the kit described herein is provided for use as a medicament or for use in the treatment or prophylaxis of a disease or disorder, preferably as defined herein, wherein the use as a medicament or the treatment or prophylaxis comprises a step of site-directed editing of a target RNA.

In one aspect, the present invention further provides a method for treating a subject suffering from a disease or a disorder, the method comprising administering an effective amount of the artificial nucleic acid, the vector, the cell or the composition described herein to the subject. An effective amount in the context of the present disclosure is typically understood to be an amount that is sufficient to trigger the desired therapeutical effect, i.e. to achieve editing of a target RNA.

The disease or the disorder may be selected from the group consisting of infectious diseases, tumour diseases, cardiovascular diseases, autoimmune diseases, allergies and neurological diseases or disorders, wherein the disease or the disorder is preferably selected from a genetic disease or genetic disorder, which is preferably selected from the group consisting of metabolic diseases, tumour diseases, autoimmune diseases, cardiovascular diseases and neurological diseases.

The artificial nucleic acid, the vector, the cell, or the (pharmaceutical) composition described herein may be administered orally, parenterally, by inhalation spray, topically, rectally, nasally, buccally, vaginally, via an implanted reservoir or via jet injection. The term parenteral as used herein includes intra-vitreal, sub-retinal, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, and sublingual injection or infusion techniques. In a preferred embodiment, the artificial nucleic acid molecule, the vector, the cell or the (pharmaceutical) composition described herein is administered via needle-free injection (e.g. jet injection).

Preferably, the artificial nucleic acid, the vector, the cell, or the (pharmaceutical) composition described herein is administered parenterally, e.g. by parenteral injection, more preferably by intra-vitreal, sub-retinal, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, sublingual injection or via infusion techniques. Particularly preferred is intradermal and intramuscular injection. Sterile injectable forms of the inventive pharmaceutical composition may be aqueous or oleaginous suspension. These suspensions may be formulated according to techniques known in the art using suitable dispersing or wetting agents and suspending agents.

The artificial nucleic acid, the vector, the cell, or the (pharmaceutical) composition described herein may also be administered orally in any orally acceptable dosage form including, but not limited to, capsules, tablets, aqueous suspensions or solutions.

The artificial nucleic acid, the vector, the cell, or the (pharmaceutical) composition described herein may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the artificial nucleic acid, the vector, the cell, or the (pharmaceutical) composition described herein may be formulated in a suitable ointment suspended or dissolved in one or more carriers.

In one embodiment, the use as a medicament comprises the step of transfection of mammalian cells, preferably in vitro or ex vivo transfection of mammalian cells, more preferably in vitro transfection of isolated cells of a subject to be treated by the medicament. If the use comprises the in vitro transfection of isolated cells, the use as a medicament may further comprise the re-administration of the transfected cells to the patient. The use of the artificial nucleic acid or the vector as a medicament may further comprise the step of selection of successfully transfected isolated cells. Thus, it may be beneficial if the vector further comprises a selection marker.

According to another aspect of the present invention, the artificial nucleic acid, the vector, the cell, or the (pharmaceutical) composition described herein is provided for use in the diagnosis of a disease or disorder, which is preferably selected from the group consisting of infectious diseases, tumour diseases, cardiovascular diseases, autoimmune diseases, allergies and neurological diseases or disorders, in particular from a genetic disease or genetic disorder, which is preferably selected from the group consisting of metabolic diseases, tumour diseases, autoimmune diseases, cardiovascular diseases and neurological diseases.

### BRIEF DESCRIPTION OF THE FIGURES

The figures shown in the following are merely illustrative and shall describe the present invention in a further way. These figures shall not be construed to limit the present invention thereto.

FIG. 1: Artificial nucleic acids used in Example 1:
A: Artificial nucleic acid molecule comprising a 20 nucleotides antisense part having a C/A mismatch at position 8, and a schematic R/G motif comprising a stem-loop structure.
B: Artificial nucleic acid molecule according to the present invention comprising a 20 nucleotides targeting sequence (TS) having a C/A mismatch at position 8, a R/G motif comprising a stem-loop structure, and a cluster of 3 recruitment sequences (RS #1, RS #2, RS #3) having 11 to 16 nucleotides which are linked via an adenosine linker (AAA).
B: Editing of the dual-luciferase W417X amber reporter using endogenous ADAR1 in HeLa cells. 120.000 cells were seeded in 24-well scale. 24 h post seeding the cells were transfected with 800 ng plasmid encoding the artificial nucleic acids A and B, respectively, and 200 ng dual-luciferase reporter per well using a plasmid to Lipofectamine-3000 ratio of 1:1.5. 72 h post transfection the cells were harvested. After RNA-isolation, DNase-I digestion and RT-PCR this was followed by sanger-sequencing.

FIG. 2: A: Artificial nucleic acids and R/G motif versions used in Example 2:
(A): Prior art design without recruitment sequence (RS) and with 16 nt antisense part and R/G motif version 20 (RG-V20).
(B): Novel design with recruitment cluster including three recruitment sequences (3×RS) and R/G motif version 21 (RG-V21).
(C): Sequences of the RG motif Version 20 and Version 21.
B: Editing in HeLa cells using endogenous human ADAR1 and Adenovirus(AdV)-encoded R/G gRNAs.
200 k HeLa cells were seeded in a 96 well scale. Reverse transduction was accomplished using 175 MOI gRNA AdV at seeding. Forward transduction was accomplished 24 h post seeding using 175 MOI dual-luciferase wt/amb AdV or 5 MOI wt/wt. Note: The amount of wt/wt AdV has no effect on the final percentage as Firefly is always normalized over Renilla. The Luciferase assay was performed 96 h after reverse infection.

FIG. 3: Editing of the dual-luciferase reporter via recruitment of endogenous human ADAR1 using AdV encoded 3×RS guideRNAs in several cell lines. Analysis via dual-luciferase assay (N=3 with two technical replicates each).
25 k cells for each cell type were seeded in a 96 well scale. In HeLa cells forward transduction was accomplished 24 h post seeding. In all other cells reverse transduction was accomplished at seeding. Harvest and luciferase assays were accomplished after 96 h post infection.
Luciferase assays: 420 Datapoints (35 settings, 6 renilla and 6 firefly per setting) Settings: gRNA MOI: HeLa (100 MOI), SK-N-BE (100 MOI), Huh7 (75 MOI), A549 (75 MOI), HepG2 (75 MOI), SY5Y (100 MOI), U87MG (100 MOI), U20S (75 MOI); DL wt/amb 50 MOI; DL wt/wt 5 MOI

FIG. 4: Editing of the dual-luciferase reporter via recruitment of endogenous human ADAR1 using AdV encoded 3×RS guideRNAs in several cell lines. Analysis via Sanger sequencing.
For Huh7, SK-N-BE, A549, HepG2 and SY5Y cells: 25.000 cells per well (96-well scale) were reverse infected with the indicated MOI at seeding on day 1. Medium change was performed on day 2-4. Harvest and pooling of 6 wells per RT-PCR was performed on day 5. For HeLa cells: 25.000 cells per well (96-well scale) were seeded on day 1. Forward infection with the following MOI was performed on day 2: HeLa: n=2 [75 MOI], n=2 [100 MOI], n=2 [125 MOI]; Huh7: n=3 [75 MOI]; A549: n=3 [75 MOI]; HepG2: n=3 [75 MOI]; SK—N-BE(2): n=3 [100 MOI]; SH-SY5Y: n=3 [100 MOI]. Medium change was performed on day 3-5. Harvest and pooling of 6 wells per RT-PCR was performed on day 6.

FIG. 5: A: Editing of several disease relevant target mRNAs in HeLa cells using 6-9×RS guideRNAs and endogenous ADAR1. 120.000 cells were seeded in 24-well scale. 24 h post seeding the cells were transfected with 800 ng guideRNA plasmid and 200 ng target encoding plasmid (cDNA) per well using a plasmid to Lipofectamine-3000 ratio of 1:1.5. 72 h post transfection the cells were harvested. After RNA-isolation, DNase-I digestion and RT-PCR this was followed by Sanger-sequencing.
B: Exemplary Sanger sequencing traces of the data displayed in FIG. 5A that show completely absent off-target bystander editing around the target site.

FIG. 6: Plasmid encoded RG-V21_20p8_3×RS and RG-V21_20p8_8×RS guideRNAs recruit mainly ADAR1 isoform p110 in HeLa cells. 1.2×10⁵ HeLa cells were seeded in a 12-well scale and reverse transfection was accomplished with scramble siRNA, ADAR1 siRNA, or ADAR1p150 siRNA using 2.5 pmol siRNA per well (3 μl HighPerfect+2.5 μl 1 μM siRNA ad 200 μl with OptiMem). 6 wells per siRNA were seeded. Each used well of the 12-well plate contained the indicated 200 μl transfection mix for the corresponding siRNA. 24 h post siRNA transfection, similarly treated wells were harvested and the cells were pooled. Then 25.000 of the differently treated HeLa cells were seeded in a 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

FIG. 7: Effect of the number of recruitment sequences on the editing yield. Luciferase assay settings of the characterization experiments: 25.000 HeLa cells were seeded in a 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system. The position of the target sequence on the Luciferase transcript is indicated in dark grey, the positions of the respective RS binding regions in light grey. The position of the editing target (5′-UAG) is also indicated. The individual RS are 9-16 nt in length and separated by an AAA linker.

FIG. 8: A: Effect of recruitment sequence length (7 to 15 nt per RS) on the editing yield. Luciferase assay settings of the characterization experiments: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.
B: Schematic representation of the minimum free energy structures of guideRNA at 37° C.

FIG. 9: A: Effect of adenosine linker length on the editing yield. Luciferase activity (fold change) is displayed next to the scheme showing the corresponding gRNAs binding regions (BR) on the mRNA. Luciferase assay settings of the characterization experiments: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.
B: Dataset of FIG. 9A, but displayed next to the scheme showing the corresponding gRNA composition with regard to the number of adenosine nucleotides used as linkers between the recruitment sequences (RS) and number of adenosine nucleotides used as spacer between the recruitment sequence #1 and the targeting sequence.

FIG. 10: Optimization of the placement of three recruitment sequences on a target mRNA. Luciferase assay settings of the characterization experiments: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system. (S=Short distance between two RS (15-62 nt); L=Long distance between two RS (375-460 nt))

FIG. 11: Optimization of the placement of an extended recruitment sequence (20 nt) in the context of two short recruitment sequences (15 nt) on a target mRNA. Luciferase assay settings of the characterization experiments: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system. (S=Short distance between two RS (15-62 nt); L=Long distance between two RS (375-460 nt); RS extended from 15 to 20 nt are highlighted)

FIG. 12: A: Effect of recruitment sequence masking (simulation of guideRNAs with strong secondary structure in the antisense part). The upper left part of FIG. 12A describes the representation used to display the interaction of gRNA and mRNA. The binding schematics part shows the binding regions (BR) of the gRNAs' recruitment sequences on the mRNA. The RS #1 binds to BR #1. The gRNA schematics show the composition of the gRNAs consisting of R/G motif V21, the targeting sequence, adenosine nucleotide linkers/spacer, the recruitment sequences (RS) #1-#3 and in some cases the masking recruitment sequences (mRS) #4-#6. Luciferase assay settings of the characterization experiments: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.
B: Schematic representation of the minimum free energy structures of guideRNA at 37° C.

FIG. 13: Editing of the dual-luciferase W417X amber reporter in C57BL/6 mice after hydrodynamic tail vein (HDTV) injection of gRNA and reporter plasmids. Negative control group mice were treated with 10 μg dual-luciferase wt/amb reporter plasmid, the positive control group mice with 10 μg dual-luciferase wt/wt reporter plasmid and the editing group mice with 5 μg dual-luciferase wt/amb reporter plasmid and 25 μg guideRNA plasmid. The guideRNA was a 20-15-15-20p8 guideRNA, thus containing three RS (with 20 nt, 15 nt, and 15 nt length) and the typical 20 nt targeting sequence with the cytosine that mismatches the adenosine in position 8. The guideRNA additionally contained the V21 R/G-motif at its 5′-terminus (not shown). Editing was analysed by the dual luciferase assay (protein level) and by Sanger sequencing after reverse transcription (RNA level). Exemplary Sanger sequencing traces are shown.

FIG. 14: Editing of endogenous UTR targets using endogenous ADAR1 in HEK293FT cells. 60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR clean-up and Sanger Sequencing (MWG). An exemplary Sanger sequencing trace is shown, the editing site and yield is indicated. “A” and “G” represent the adenosine and guanosine peak, respectively.

FIG. 15: Editing of endogenous ORF targets using endogenous ADAR1 in HEK293FT cells. 60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR clean-up and Sanger Sequencing (MWG). An exemplary Sanger sequencing trace is shown, the editing site and yield is indicated. “A” and “G” represent the adenosine and guanosine peak, respectively.

FIG. 16: A: Benchmark comparing the guideRNA of the present invention with prior art LEAPER gRNAs targeting NUP43 V233V. Editing of endogenous targets using endogenous ADAR1 in HEK293FT cells. 60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR cleanup and Sanger Sequencing (MWG). N=3 biological replicates.
B: Benchmark comparing the guideRNA of the present invention with prior art LEAPER gRNAs targeting RAB7A 3′UTR TAG #1. Editing of endogenous targets using endogenous ADAR1 in HEK293FT cells. 60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR cleanup and Sanger Sequencing (MWG). N=3 biological replicates.

FIG. 17: Recruitment Cluster In-Silico optimization using the recruitment cluster finder tool

FIG. 18: A: Legend for FIG. 18B and FIG. 18C. RM=Recruiting moiety.
B: The recruitment sequences (RS) and the double stranded ADAR recruiting domains (e.g. R/G motif) can be arranged around the targeting sequence (TS) with certain flexibility. The components of the antisense part of a CLUSTER guide RNA targeting the dual luciferase reporter in HeLa cells were newly arranged, starting from the conventional #6-#5-#4-TS design.
C: Results of the dual-luciferase assay. Data are shown as the mean±s.d. of N=5 biological replicates. Notably, some designs, e.g. #4-TS-#3-#2 gave comparably good editing yields to the conventional #6-#5-#4-TS design. Importantly, the former design allows to include the sequence space 3′ of the targeted adenosine (e.g. binding sites #3, #2, #1) for the binding of the CLUSTER guide RNA. Luciferase assay settings of this experiment: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

FIG. 19: A: Correction of the disease relevant hIDUA W402X amber mutation in primary fibroblasts from a Hurler syndrome patient (Hurler FB, GM06214) via transfection of a chemically synthesized CLUSTER antisense oligonucleotide (ASO). The editing yield was determined by Sanger sequencing. Fibroblasts from patients with Hurler syndrome (GM06214) were purchased from the Coriell Institute for Medical Research (USA). 2.5×10⁵ cells/well in 2.5 ml DMEM+15% FBS were seeded into 6-well plates. For each tested condition one 6-well was used to determine RNA editing yields by Sanger sequencing. The CLUSTER ASO was a PAGE-purified, endblocked (2′-OMe, PS) RNA oligonucleotide with a 3×RS (20-20p8-25-20) CLUSTER design, which was ligated (T4 RNA ligase) inhouse from two commercially purchased (Biospring GmbH, Germany) and HPLC purified oligonucleotides of 69 nt (5′part) and 80 nt (3′part) length, according to our recently published protocol. The full sequence and modification pattern is given in the list if applied gRNAs below. Transfection was performed 24 h after seeding with 125 pmol ASO and 7.5 μl RNAiMAX, each diluted in 250 μl Opti-MEM. Both solutions were combined after 5 min incubation and incubated for an additional 20 min before the transfection mix was distributed evenly into one well. The medium was changed 24 h after transfection. 48 h after transfection, fibroblasts were harvested in RLT-buffer (QIAGEN, #79216), followed by RNA isolation using the Monarch RNA clean up kit (NEB, (#T2030L). Turbo-DNase digestion was performed using the TURBO DNA-free Kit (Thermofisher, #AM1907). Reverse transcription was performed using the SuperScript IV RT (Thermofisher, #18090050) with random primers for 1 h at 52° C. A regular PCR followed by a nested PCR was performed using Taq Polymerase (NEB, #M0267S) with each reaction containing 10% DMSO. After the second PCR the products were separated by SB-agarose gel-electrophoresis. After PCR cleanup (NucleoSpin Gel and PCR Clean-up Kit, Macherey Nagel, #740609), Sanger sequencing (Microsynth AG) was performed.
B: In addition, the restoration of protein function was determined using the α-L-iduronidase enzyme activity assay. The measured hIDUA enzyme activities in the Hurler fibroblasts were normalized to the IDUA enzyme activity measured in Scheie patient fibroblasts (Scheie FB, GM01323). Data are shown as the mean±s.d. of N=4-5 biological replicates, as indicated by the individual data points. Fibroblasts from patients with Scheie syndrome (GM01323) and Hurler syndrome (GM06214) were purchased from the Coriell Institute for Medical Research (USA). 2.5×10⁵ cells/well in 2.5 ml DMEM+15% FBS were seeded into 6-well plates. For each tested condition, two 6-wells were used for the IDUA assay. The CLUSTER ASO was a PAGE-purified, endblocked (2′-OMe, PS) RNA oligonucleotide with a 3×RS (20-20p8-25-20) CLUSTER design, which was ligated (T4 RNA ligase) inhouse from two commercially purchased (Biospring GmbH, Germany) and HPLC purified oligonucleotides of 69 nt (5′part) and 80 nt (3′part) length, according to our recently published protocol. The full sequence and modification pattern is given in the list if applied gRNAs below. Transfection was performed 24 h after seeding with 125 pmol ASO and 7.5 μl RNAiMAX, each diluted in 250 μl Opti-MEM. Both solutions were combined after 5 min incubation and incubated for an additional 20 min before the transfection mix was distributed evenly into one well. The medium was changed 24 h after transfection. 48 h after transfection, fibroblasts were detached and washed once with PBS. 40 μl 0.5% Triton X-100 in PBS were added to the cell pellet and incubated on ice for 30 min and α-L-iduronidase enzyme assay was performed. For the editing read-out via α-L-iduronidase enzyme activity assay a standard dilution series of 4-methylumbelliferone (Sigma Aldrich, M1381) was prepared in 1× PBS. For each concentration, 25 μl of the standard solution were added to 25 d 0.4 M sodium formate buffer (pH 3.5) and applied to a 96-well LumiNunc plate (VWR, 732-2696) in triplicate. The substrate (4-methylumbelliferyl α-L-iduronide, Glycosynth, #44076) was dissolved in 0.4 M sodium formate buffer to a final concentration of 180 μM. For the murine IDUA assay using HeLa cells 25 μl of the 1:3 diluted cell lysate (0.5% Tween-20/PBS) was added to 25 μl substrate in the plate and incubated for 45 min at 37° C. in the dark. 25 μl of undiluted cell lysate (0.5% Triton X-100/PBS) was added to 25 μl substrate in the plate and incubated for 90 min at 37° C. in the dark. The reaction was quenched in both cases by adding 200 μl glycine carbonate buffer (0.17 M Glycine/NaOH, pH 10.4). The fluorescence of 4-methylumbelliferone was measured with an excitation wavelength of 355 nm at an emission wavelength of 460 nm with a Tecan Spark 10M plate reader. Calculated enzyme activities were referenced to the protein amount as determined by BCA assay (Pierce BCA Protein Assay Kit, Thermofisher, 23227). The enzyme activity was standardized to Scheie fibroblast lysate.

FIG. 20: A: Analysis of off-target editing in the poly(A)+transcriptome for recruiting endogenous ADAR from 293FT cells to a 5′ UAG site in the 3′-UTR of endogenous RAB7A with a 19-11-13-20p8 CLUSTER guide RNA. The scatter plot shows differential editing at ˜30,000 sites comparing editing levels in cells transfected with a plasmid carrying the CLUSTER or a non-targeting guide RNA. Experiments were done with two independent replicates. The on-target editing is indicated by an arrow. Significantly differently edited sites (adjusted P<0.01, Fisher's exact test, two-tailed, N 50) are highlighted in black. The RNA editing experiment was done by transfection of 1200 ng guide RNA plasmid (NucleoSpin Plasmid Transfection-grade, Macherey Nagel, #740490) into 6×10⁴ HEK293FT cells 24 h post seeding using a 1:3 ratio of FuGene6 (Promega, #E2691) in 24 cell format. 48 h after transfection, cells were harvested. Overall, three settings were carried out, each with an independent duplicate. Those settings include a non-targeting guide RNA (NT-RNA) and a RAB7A 3′UTR 19-11-13-20p8 CLUSTER guide RNA. RNA was isolated with the RNeasy MinElute Kit (Qiagen, #74204), treated with DNase 1 (NEB, #M0303S), incubated with an RNA strand reverse complementary to the antisense part of the respective guide RNA, heated to 95° C. for 3 min and purified again with the RNeasy MinElute Kit. Purified RNA was delivered to CeGaT (Germany) for poly(A)+mRNA sequencing. The library was prepared from 200 ng RNA with the TruSeq Stranded mRNA Library Prep Kit (Illumina, USA) and sequenced with a NovaSeq 6000 (50M reads, 2×100 bp paired end, Illumina, USA).
B: Analysis of off-target editing in the poly(A)+transcriptome for recruiting endogenous ADAR from 293FT cells to a 5′ UAG site in the 3′-UTR of endogenous RAB7A with a 111 nt LEAPER guide RNA. The scatter plot shows differential editing at ˜30,000 sites comparing editing levels in cells transfected with a plasmid carrying the LEAPER or a non-targeting guide RNA. Experiments were done with two independent replicates. The on-target editing is indicated by an arrow. Significantly differently edited sites (adjusted P<0.01, Fisher's exact test, two-tailed, N 50) are highlighted in black. The RNA editing experiment was done by transfection of 1200 ng guide RNA plasmid (NucleoSpin Plasmid Transfection-grade, Macherey Nagel, #740490) into 6×104 HEK293FT cells 24 h post seeding using a 1:3 ratio of FuGene6 (Promega, #E2691) in 24 cell format. 48 h after transfection, cells were harvested. Overall, three settings were carried out, each with an independent duplicate. Those settings include a non-targeting guide RNA (NT-RNA) and a RAB7A 3′UTR 111p56 LEAPER guide RNA. RNA was isolated with the RNeasy MinElute Kit (Qiagen, #74204), treated with DNase I (NEB, #M0303S), incubated with an RNA strand reverse complementary to the antisense part of the respective guide RNA, heated to 95° C. for 3 min and purified again with the RNeasy MinElute Kit. Purified RNA was delivered to CeGaT (Germany) for poly(A)+mRNA sequencing. The library was prepared from 200 ng RNA with the TruSeq Stranded mRNA Library Prep Kit (Illumina, USA) and sequenced with a NovaSeq 6000 (50M reads, 2×100 bp paired end, Illumina, USA).
C: Assessing the editing precision by analyzing all NGS reads with on-target editing for bystander editing. While CLUSTER guide RNAs give mainly clean sequencing reads and a small fraction of reads with single bystander edits, reads from the LEAPER sample very frequently contained several bystander edits. The RNA editing experiment was done by transfection of 1200 ng guide RNA plasmid (NucleoSpin Plasmid Transfection-grade, Macherey Nagel, #740490) into 6×104 HEK293FT cells 24 h post seeding using a 1:3 ratio of FuGene6 (Promega, #E2691) in 24 cell format. 48 h after transfection, cells were harvested. Overall, three settings were carried out, each with an independent duplicate. Those settings include (1) non-targeting guide RNA (NT-RNA), (2) RAB7A 3′UTR 19-11-13-20p8 CLUSTER guide RNA, and (3) RAB7A 3′UTR 111p56 LEAPER guide RNA. RNA was isolated with the RNeasy MinElute Kit (Qiagen, #74204), treated with DNase 1 (NEB, #M0303S), incubated with an RNA strand reverse complementary to the antisense part of the respective guide RNA, heated to 95° C. for 3 min and purified again with the RNeasy MinElute Kit. Purified RNA was delivered to CeGaT (Germany) for poly(A)+mRNA sequencing. The library was prepared from 200 ng RNA with the TruSeq Stranded mRNA Library Prep Kit (Illumina, USA) and sequenced with a NovaSeq 6000 (50M reads, 2×100 bp paired end, Illumina, USA).
D: Estimation of the yield of clean editing (no bystander) versus total on-target editing. The RNA editing experiment was done by transfection of 1200 ng guide RNA plasmid (NucleoSpin Plasmid Transfection-grade, Macherey Nagel, #740490) into 6×104 HEK293FT cells 24 h post seeding using a 1:3 ratio of FuGene6 (Promega, #E2691) in 24 cell format. 48 h after transfection, cells were harvested. Overall, three settings were carried out, each with an independent duplicate. Those settings include (1) non-targeting guide RNA (NT-RNA), (2) RAB7A 3′UTR 19-11-13-20p8 CLUSTER guide RNA, and (3) RAB7A 3′UTR 111p56 LEAPER guide RNA. RNA was isolated with the RNeasy MinElute Kit (Qiagen, #74204), treated with DNase I (NEB, #M0303S), incubated with an RNA strand reverse complementary to the antisense part of the respective guide RNA, heated to 95° C. for 3 min and purified again with the RNeasy MinElute Kit. Purified RNA was delivered to CeGaT (Germany) for poly(A)+mRNA sequencing. The library was prepared from 200 ng RNA with the TruSeq Stranded mRNA Library Prep Kit (Illumina, USA) and sequenced with a NovaSeq 6000 (50M reads, 2×100 bp paired end, Illumina, USA).

FIG. 21: List of off-target events induced by RNA editing with CLUSTER- and LEAPER-guide RNAs determined by next generation sequencing. This list shows the subset of off-target editing events at “unknown” sites that have been detected by the pipeline searching for significantly differentially edited sites in at least one sample when cells were treated either with the CLUSTER or the LEAPER guide RNA and compared to the non-targeting control guide RNA (NT gRNA). Sites were assigned “unknown” when they were not listed in the RADAR database. Nonsynonymous editing was only detected for a single site, HTATSF1 (S742G). Mapping analysis detected sites for potential off-target binding of the RAB7A guide RNAs to CTNNAL1, HTATSF1, and ZNF740. The detected RAB7A sites represent the fraction of bystander editing sites close to the on-target site that met the significance and cut-off criteria of the pipeline. Data are representing the mean±s.d. of N=2 NGS replicates. The RNA editing experiment was done by transfection of 1200 ng guide RNA plasmid (NucleoSpin Plasmid Transfection-grade, Macherey Nagel, #740490) into 6×10⁴ HEK293FT cells 24 h post seeding using a 1:3 ratio of FuGene6 (Promega, #E2691) in 24 cell format. 48 h after transfection, cells were harvested. Overall, three settings were carried out, each with an independent duplicate. Those settings include (1) non-targeting guide RNA (NT-RNA), (2) RAB7A 3′UTR 19-11-13-20p8 CLUSTER guide RNA, and (3) RAB7A 3′UTR 111p56 LEAPER guide RNA. RNA was isolated with the RNeasy MinElute Kit (Qiagen, #74204), treated with DNase I (NEB, #M0303S), incubated with an RNA strand reverse complementary to the antisense part of the respective guide RNA, heated to 95° C. for 3 min and purified again with the RNeasy MinElute Kit. Purified RNA was delivered to CeGaT (Germany) for poly(A)+mRNA sequencing. The library was prepared from 200 ng RNA with the TruSeq Stranded mRNA Library Prep Kit (Illumina, USA) and sequenced with a NovaSeq 6000 (50M reads, 2×100 bp paired end, Illumina, USA).

### EXAMPLES

The examples shown in the following are merely illustrative and shall describe the present invention in a further way. These examples shall not be construed to limit the present invention thereto.

If not stated otherwise, the nucleic acid sequences provided herein are printed from 5′ to 3′. In other terms, the first nucleotide residue in a nucleic acid sequence printed herein is—if not stated otherwise—the 5′-terminus of said nucleic acid sequence. Amino acid sequences—if not stated otherwise—are printed from the N-terminus to the C-terminus.

### Example 1: Editing of the Dual-Luciferase W417X Amber Reporter Using Endogenous ADAR1 in HeLa Cells

The general structure of the artificial nucleic acids used in Example 1 are shown in FIG. 1A:

A: Prior art artificial nucleic acid molecule comprising a 20 nucleotides antisense part having a C/A mismatch at position 8, and a schematic R/G motif having a stem-loop structure.

B: Artificial nucleic acid molecule according to the present invention comprising a 20 nucleotides antisense part having a C/A mismatch at position 8, a R/G motif having a stem-loop structure, and a cluster of 3 recruitment sequences (RS #1, RS #2, RS #3) having 11 to 16 nucleotides which are linked via an adenosine linker (AAA).

120.000 HeLa cells were seeded in 24-well scale. 24 h post seeding the cells were transfected with 800 ng RNA plasmid encoding the artificial nucleic acid A and B, respectively, as well as an artificial nucleic acid comprising a 40 nt antisense part, and an artificial nucleic acid comprising a cluster of 8 recruitment sequences, and 200 ng dual-luciferase reporter per well using a plasmid to Lipofectamine-3000 ratio of 1:1.5. 72 h post transfection the cells were harvested. After RNA-isolation, DNase-I digestion and RT-PCR this was followed by Sanger-sequencing.

As shown in FIG. 1B, prior art 20 nt short R/G guideRNAs without recruitment sequences (RS) cannot efficiently recruit endogenous ADAR1 in HeLa cells. The addition of three recruitment sequences to a guideRNA with a 20 nt targeting sequence increases editing drastically from 0% to 20%. The addition of eight recruitment sequences to a guideRNA with a 20 nt targeting sequence increases editing drastically from 0% to 24%. Treatment of the HeLa cells with IFNa increases the editing slightly.

### Example 2: Editing in HeLa Cells Using Endogenous Human ADAR1 and AdV-Encoded R/G gRNAs

In a further experiment, RNA editing in HeLa cells was studied using a prior art guideRNA design comprising an R/G-V20 motif and a 16 nucleotide targeting sequence, and an artificial nucleic acid according to the present invention comprising an R/G-V21 motif, a 20 nucleotides targeting sequence and a recruitment cluster comprising 3 recruitment sequences having 11 to 16 nucleotides which are linked by a triple adenosine linker (AAA). Please note that the regions on the target RNA which are bound by the recruitment sequences (RS #1, RS #2, RS #3) of the artificial nucleic acid are separated by 10 to 100 nucleotides on the target RNA (see FIG. 2A).

200 k HeLa cells were seeded in a 96 well scale. Reverse transduction was accomplished using 175 MOI (multiplicity of infection) gRNA AdV at seeding. Forward transduction was accomplished 24 h post seeding using 175 MOI dual-luciferase wt/amb AdV or 5 MOI wt/wt. The Luciferase assay was performed 96 h after reverse infection.

As shown in FIG. 2B, the artificial nucleic acid according to the present invention which comprises a recruiting moiety comprising 3 recruitment sequences (RS #1, RS #2, RS #3) which bind to 11-16 nucleotide regions on the target RNA, restores luciferase activity to 100%, whereas the prior art design lacking the recruitment sequences is able to restore the (normalized) luciferase activity only to 1.5%.

### Example 3: Editing of the Dual-Luciferase Reporter Via Recruitment of Endogenous Human ADAR1 Using Adenovirusencoded 3×RS guideRNAs in Several Cell Lines

In order to evaluate editing of the dual-luciferase reporter via recruitment of endogenous human ADAR1 using Adenovirus (AdV) encoded 3×RS guideRNAs in several cell lines, 25 k cells for each cell type were seeded in a 96 well scale. In HeLa cells forward transduction was accomplished 24 h post seeding. In all other cells reverse transduction was accomplished at seeding. Harvest and luciferase assays were accomplished after 96 h post infection.

As it is shown in FIG. 3, restoration of firefly luciferase activity by RNA editing could be achieved in several cell lines deriving from different tissues (Brain, Liver, Lung, Cervix) by harnessing the endogenous ADAR1 only. In HeLa cells the normalized firefly luciferase activity could be restored up to 90% of the wild-type level. The readout was a luciferase assay.

### Example 4: Editing of the Dual-Luciferase Reporter in Several Cell Lines Using AdV Encoded 3×RS guideRNAs and Endogenous ADAR1

For Huh7, SK-N-BE, A549, HepG2 and SY5Y cells: 25.000 cells per well (96-well scale) were reverse infected with the indicated MOI at seeding on day 1. Medium change was performed on day 2-4. Harvest and pooling of 6 wells per RT-PCR was performed on day 5. For HeLa cells: 25.000 cells per well (96-well scale) were seeded on day 1. Forward infection with the indicated MOI was performed on day 2. Medium change was performed on day 3-5. Harvest and pooling of 6 wells per RT-PCR was performed on day 6.

As it is shown in FIG. 4, editing could be achieved in several cell lines deriving from different tissues (Brain, Liver, Lung, Cervix) by using only endogenous ADAR1. This time the readout was Sanger sequencing (RNA-level). However, the results are in line with restoration of luciferase activity (protein level) shown in Example 3, FIG. 3.

### Example 5: Editing of Several Disease Relevant Target mRNAs in HeLa Cells Using 6-9×RS guideRNAs and Endogenous ADAR1

In order to evaluate the therapeutic potential of artificial nucleic acids (guideRNAs) of the present invention, the editing of the following disease relevant target mRNAs in HeLa cells using 6-9×RS guideRNAs and endogenous ADAR1 was tested. The target mRNAs were encoded as cDNAs on pcDNA3 expression plasmids.

BMPR2: Mutations in bone morphogenetic protein receptor Type II (BMPR2) are the commonest genetic cause of pulmonary arterial hypertension.

COL3A1: Mutations in COL3A1 have been identified to underlie the Ehlers-Danlos syndrome type IV which is an autosomal dominant connective tissue disease.

FANCC: Diseases associated with FANCC (Fanconi anaemia complementation group C) mutations include Fanconi anaemia, complementation group C and Fanconi anaemia, complementation group A.

AHI: AHI1 specifically encodes the Jouberin protein, and mutations in the expression of the gene are known to cause specific forms of Joubert syndrome.

MYBPC: Mutations in cardiac myosin binding protein C (MyBP-C, encoded by MYBPC3) are the most common cause of hypertrophic cardiomyopathy.

IL2RG: Severe combined immunodeficiency (SCID) is a syndrome of profoundly impaired cellular and humoral immunity. In humans, SCID is most commonly caused by mutations in the X-linked gene IL2RG, which encodes the common gamma chain, gamma c, of the leukocyte receptors for interleukin-2 and multiple other cytokines.

PINK1: PTEN-induced kinase 1 (PINK1) is a mitochondrial serine/threonine-protein kinase encoded by the PINK1 gene. It is thought to protect cells from stress-induced mitochondrial dysfunction. Mutations in this gene cause one form of autosomal recessive early-onset Parkinson's disease.

120.000 cells were seeded in 24-well scale. 24 h post seeding the cells were transfected with 800 ng guideRNA plasmid and 200 ng target encoding plasmid (cDNA) per well using a plasmid to Lipofectamine-3000 ratio of 1:1.5. 72 h post transfection the cells were harvested. After RNA-isolation, DNase-I digestion and RT-PCR this was followed by Sanger-sequencing.

As shown in FIG. 5A, editing at the target site was detected in all disease relevant target mRNAs. The on-target editing level varied from 5-15% in the PINK1_W437X, IL2RG_W237X, and MYBPC3_W1098X mRNAs, to 30% in the AHI_W725X and FANCC_W506X mRNAs, and up to 55-60% in the COL3A1_W1278X and BMPR2_W298X mRNAs. Notably, even though editable adenosine nucleotides where present in close proximity of the target adenosine no bystander off-target editing was detected by Sanger sequencing. Four exemplary Sanger sequencing traces demonstrating this are given in FIG. 5B.

### Example 6: Plasmid Encoded RG-V21 20p8 3×RS and RG-V21 20p8 8×RS guideRNAs Recruit Mainly ADAR1 p110 in HeLa Cells

In order to investigate which ADAR isotype is mainly recruited by the artificial nucleic acids (guideRNAs) of the present invention, 1.2×10⁵ HeLa cells were seeded in a 12-well scale and reverse transfection was accomplished with scramble siRNA, ADAR1 siRNA, or ADAR1p150 siRNA using 2.5 pmol siRNA per well (3 μl HighPerfect+2.5 μl 1 μM siRNA ad 200 μl with OptiMem). 6 wells per siRNA were seeded. Each used well of the 12-well plate contained the indicated 200 μl transfection mix for the corresponding siRNA. 24 h post siRNA transfection, similarly treated wells were harvested and the cells were pooled. Then 25.000 of the differently treated HeLa cells were seeded in a 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

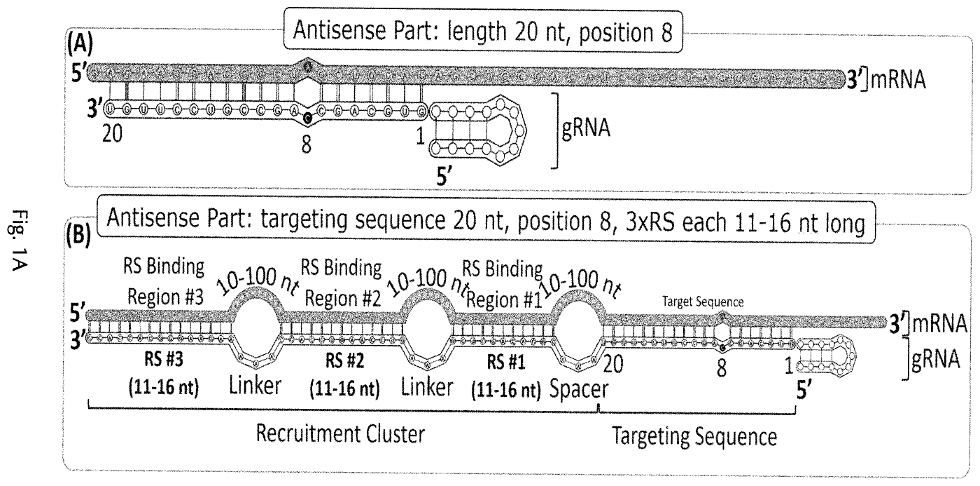

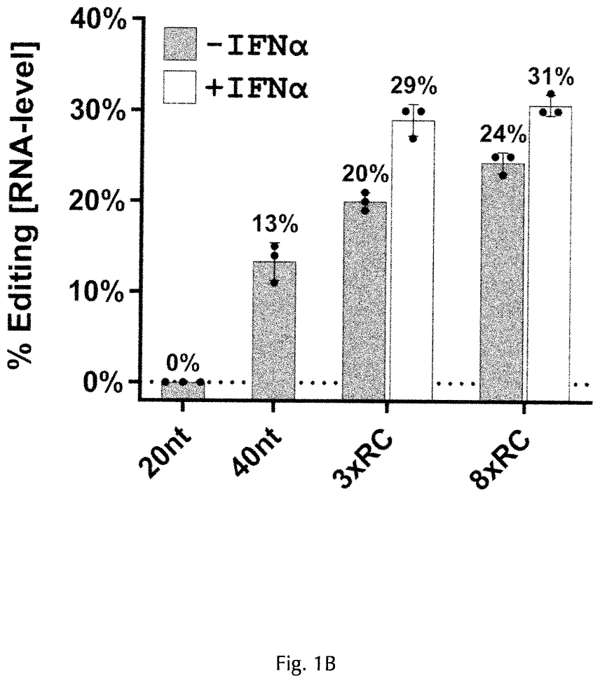

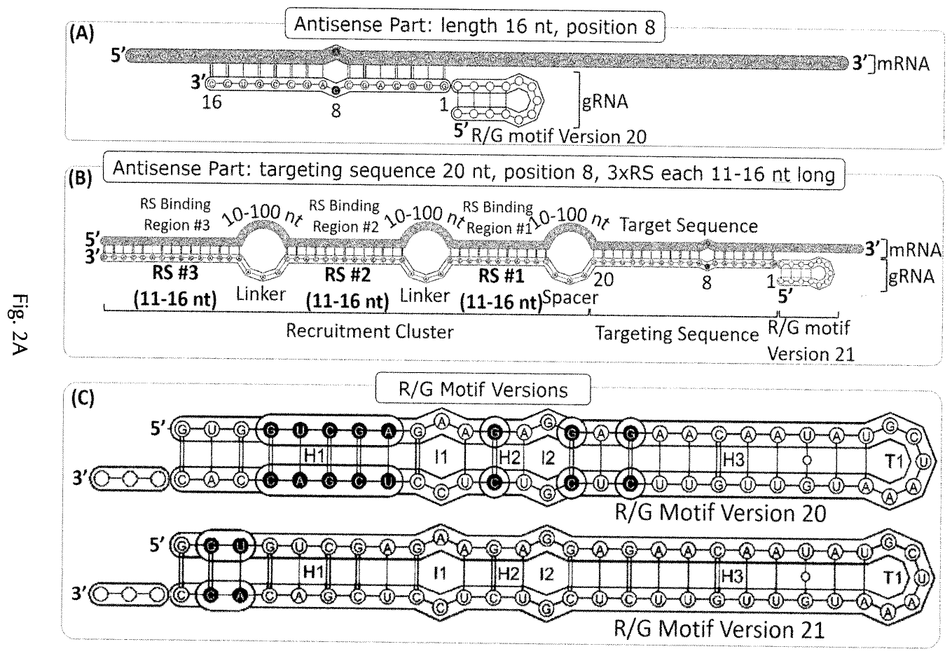

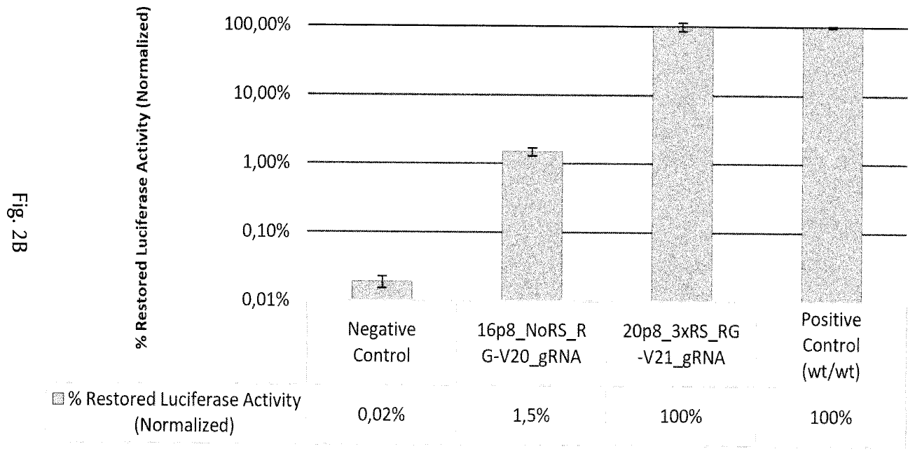

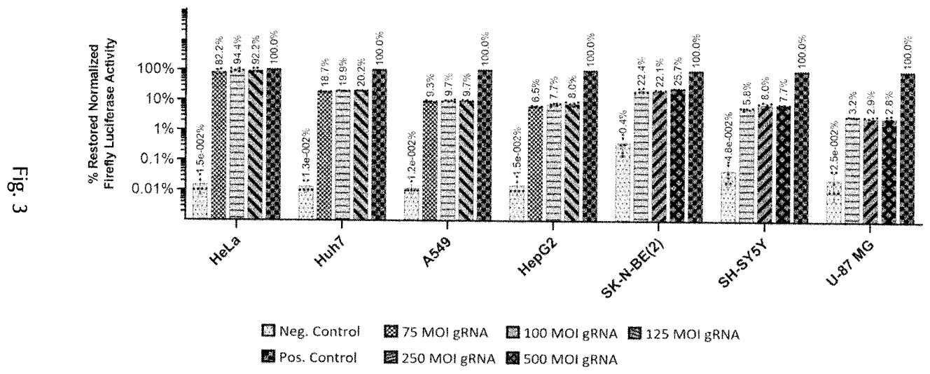

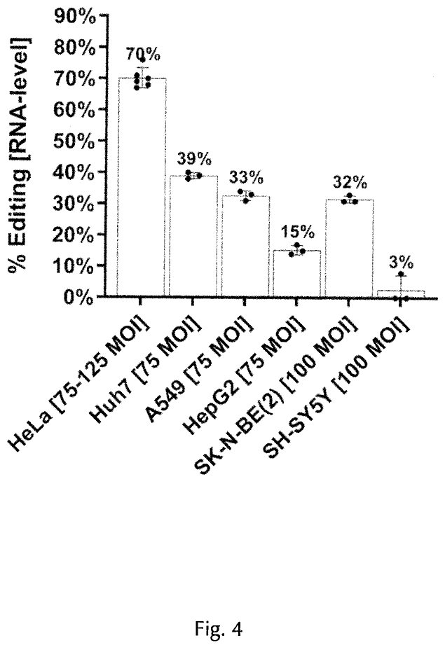

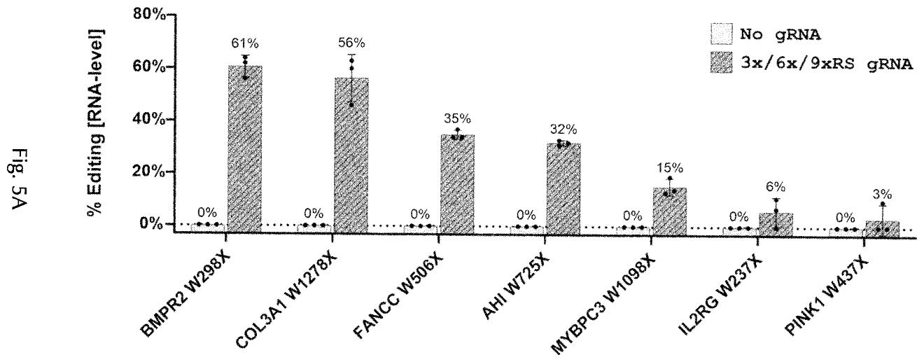

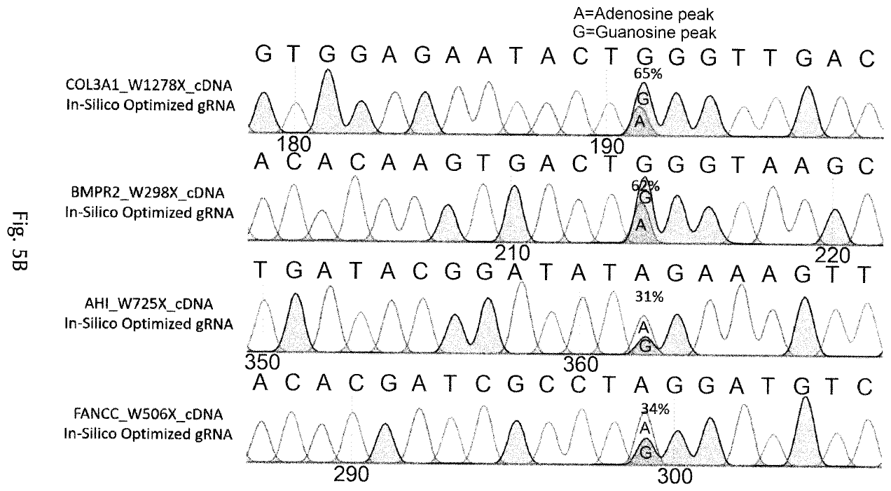

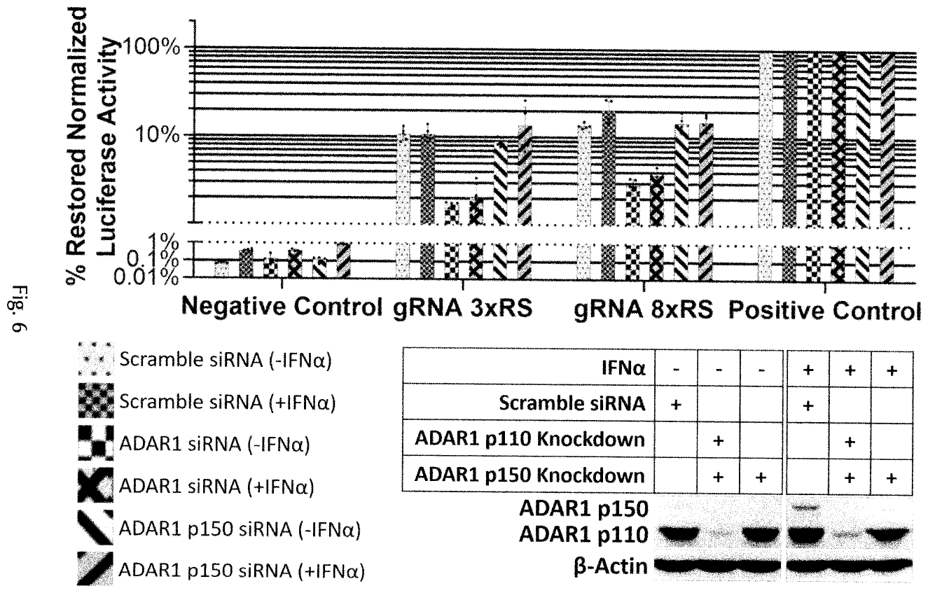

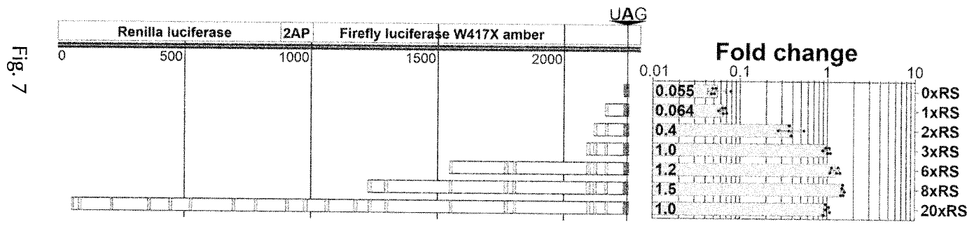

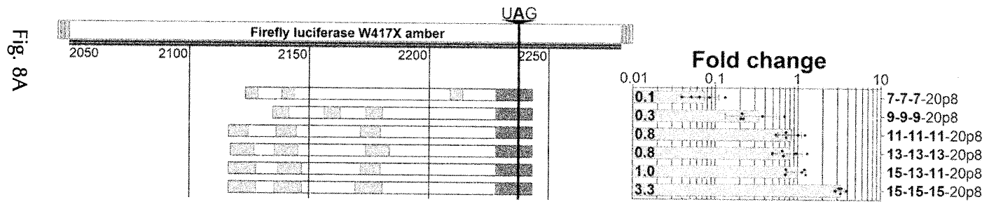

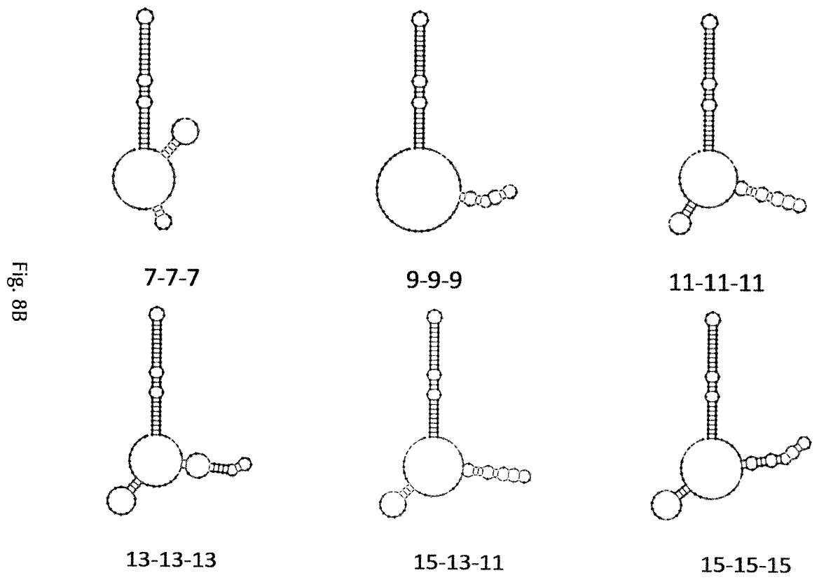

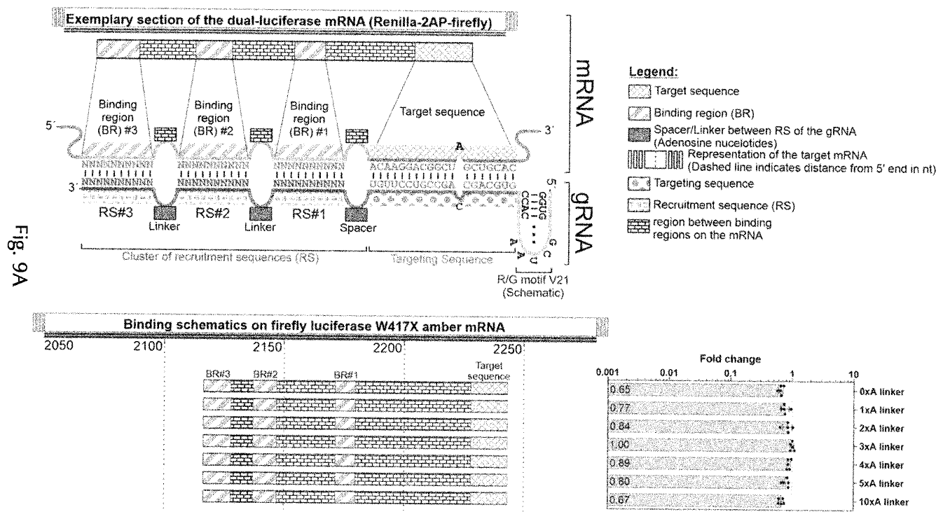

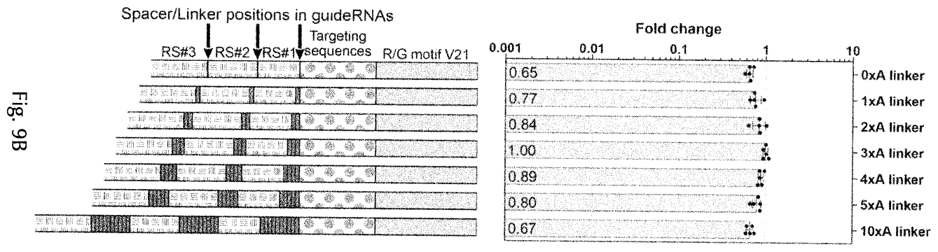

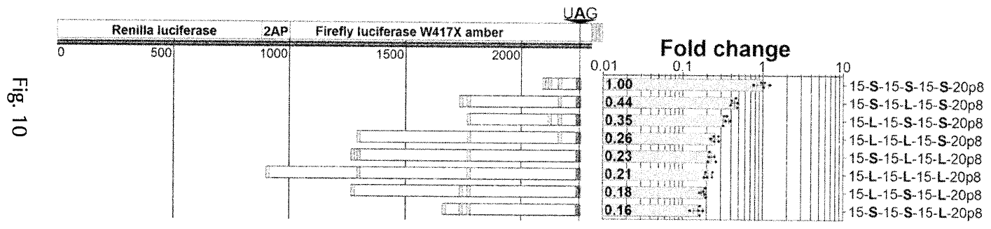

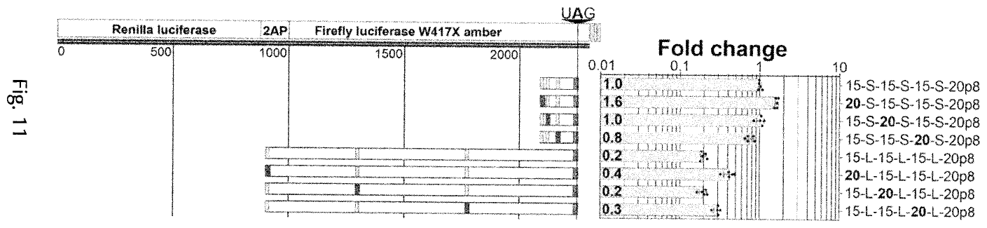

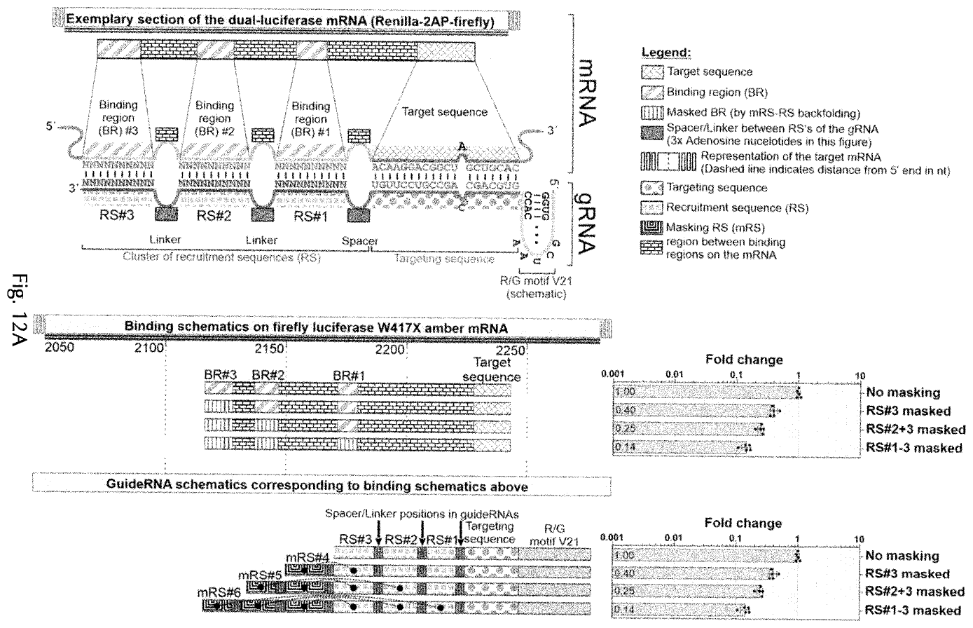

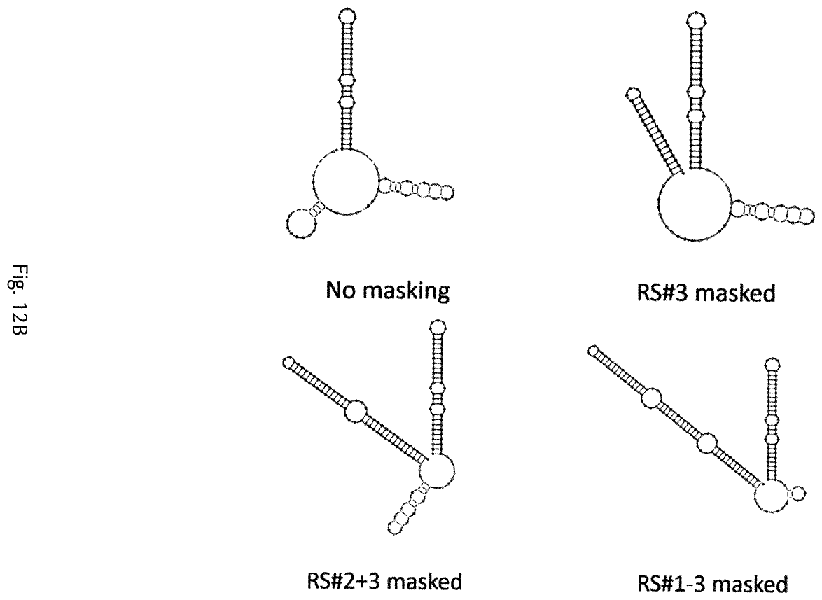

As shown in FIG. 6, an siRNA knockdown of ADAR1 (isoform p110 and p150) reduced editing nearly 10-fold in the luciferase assay, while a specific ADAR1 p150 knockdown had only minimal effect on the editing yield. Interferon-α induction of the cells did hardly increase editing yields, supporting that editing is rather performed by the constitutively expressed ADAR1 p110 than by the IFN-α inducible ADAR1 p150 isoform. As seen in the corresponding western blot the knock-down was very efficient, resulting in almost complete elimination of the targeted protein(s).

### Example 7: Effect of the Number of Recruitment Sequence on the Editing Yield

In order to investigate the effect of the number of recruitment sequences comprised in the recruiting moiety on the editing yield, several artificial nucleic acids (guideRNAs) were constructed having a recruiting moiety comprising 0, 1, 2, 3, 6, 8, or 20 recruitment sequences, a 20 nt targeting sequence and a RG-V21 motif, respectively. The RS were between 9-16 nt in length and connected to each other by triple-adenosine linkers.

25.000 HeLa cells were seeded in a 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

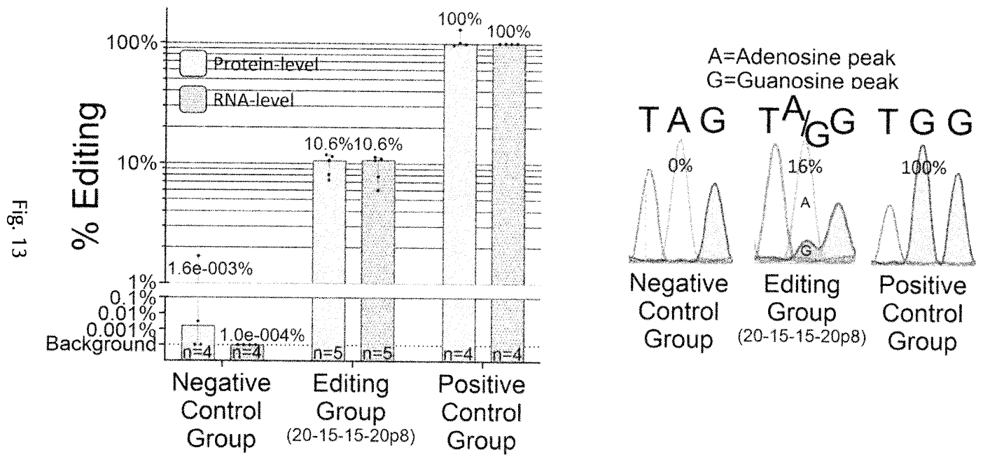

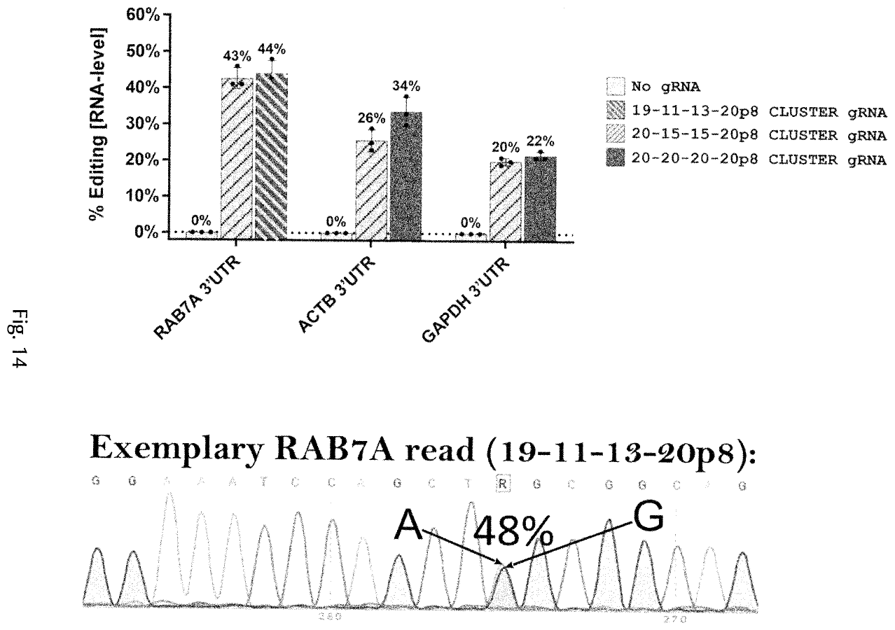

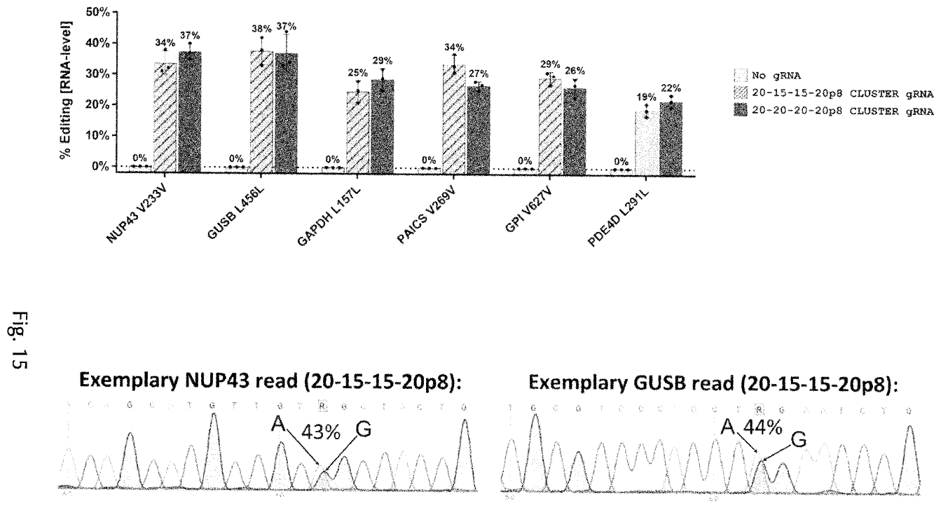

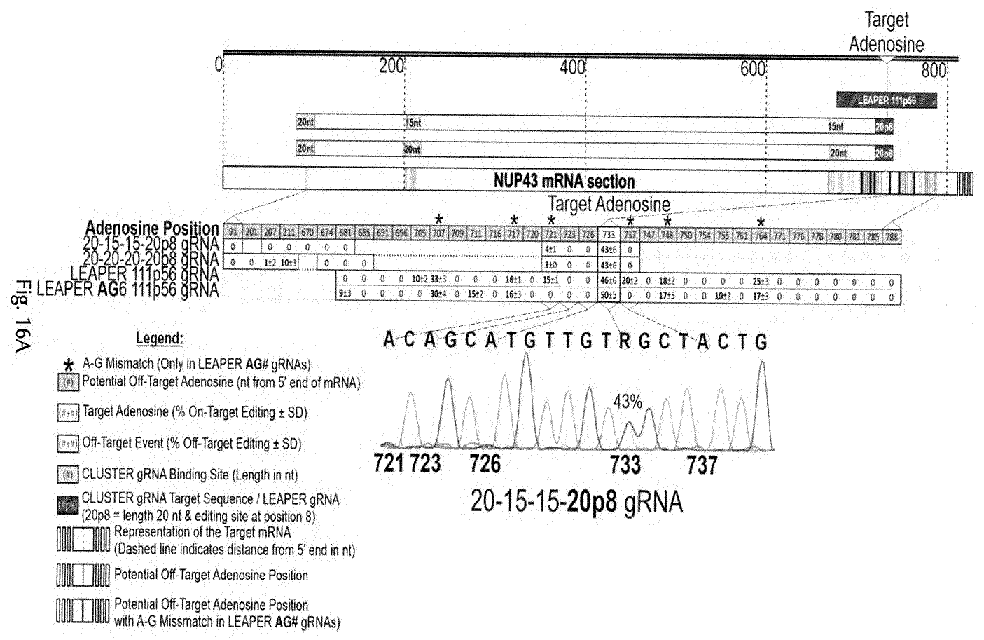

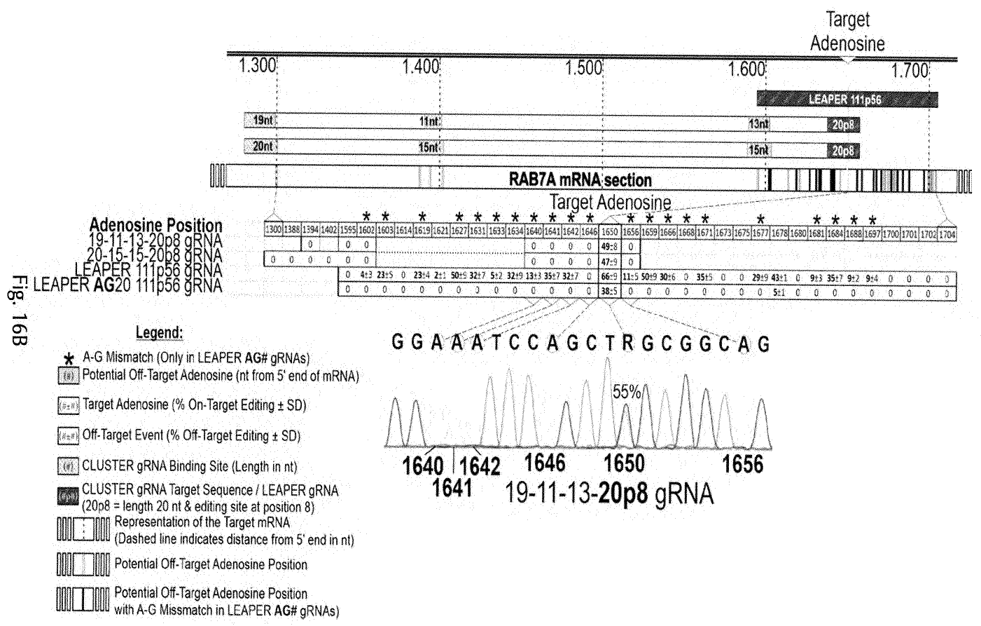

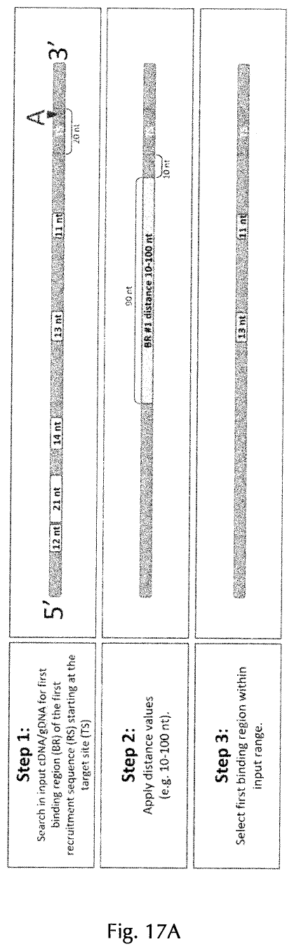

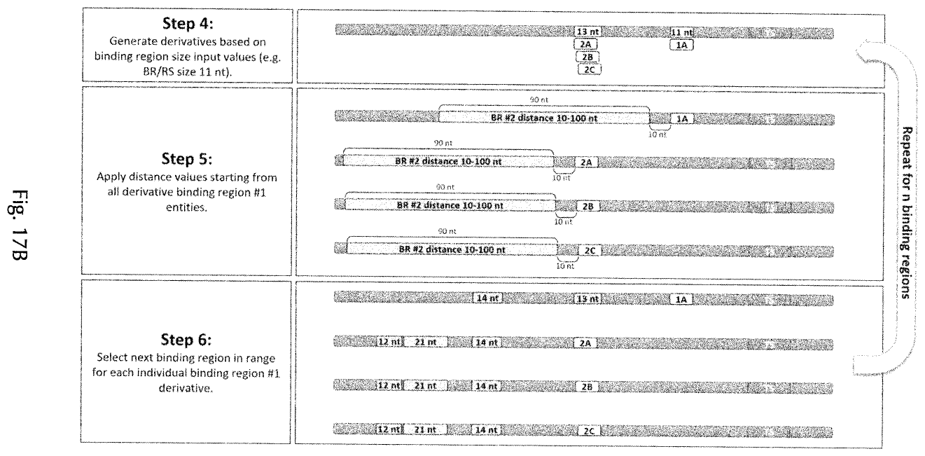

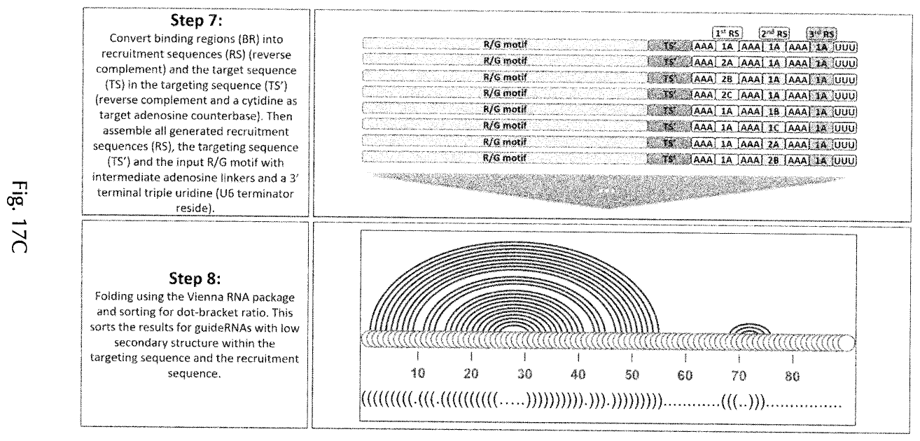

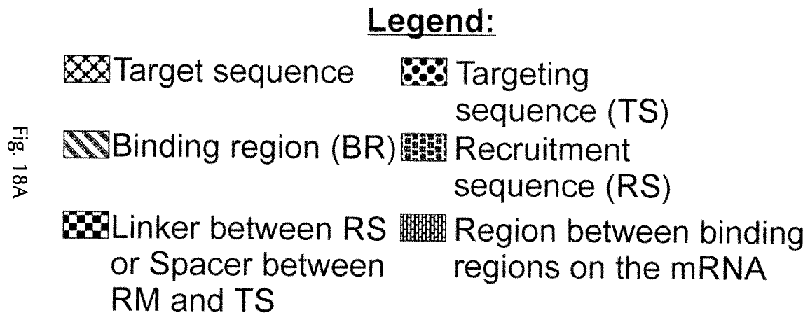

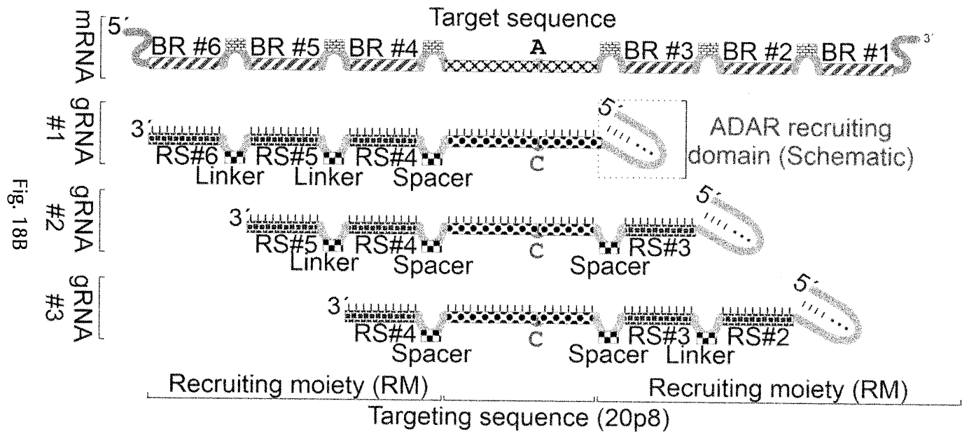

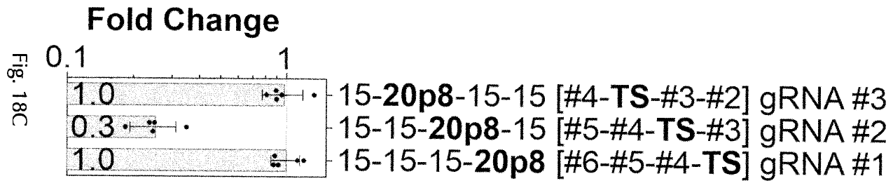

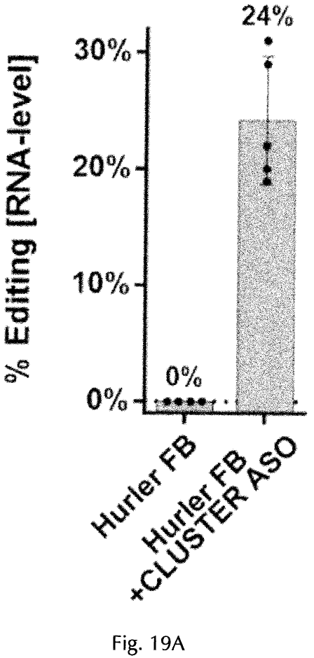

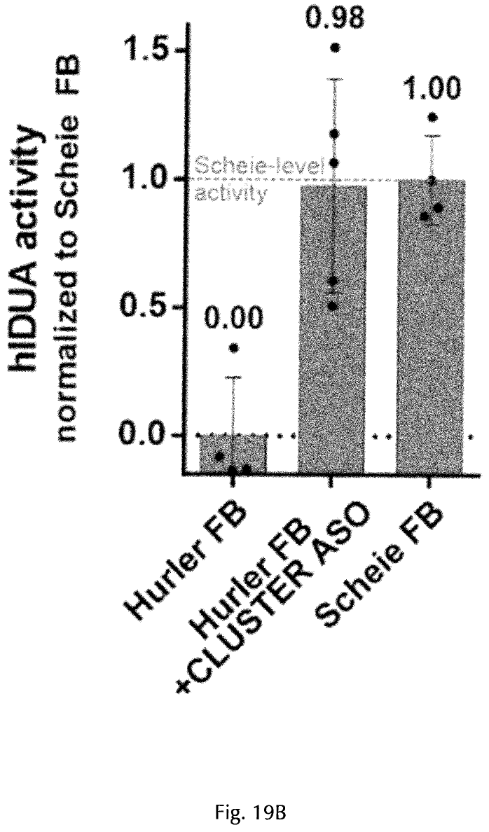

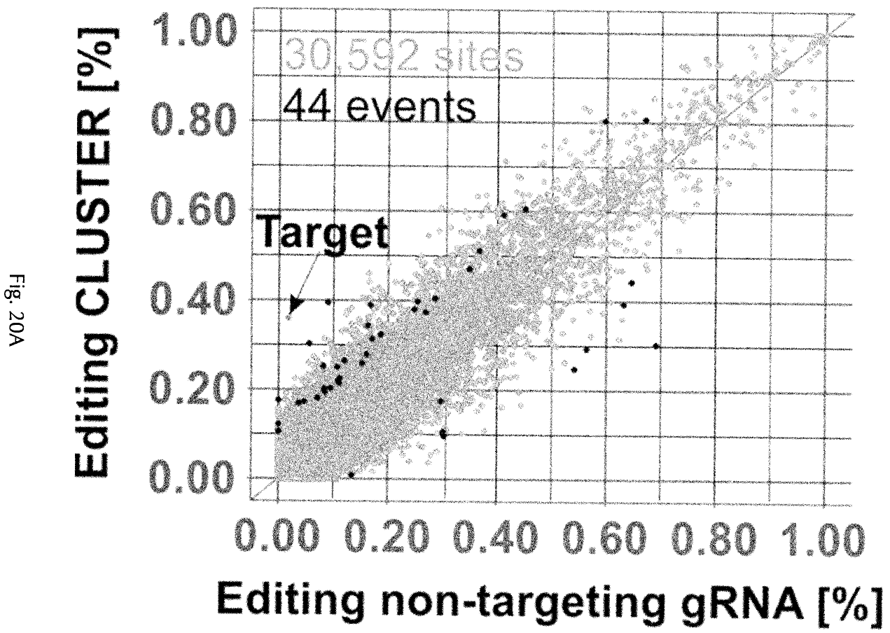

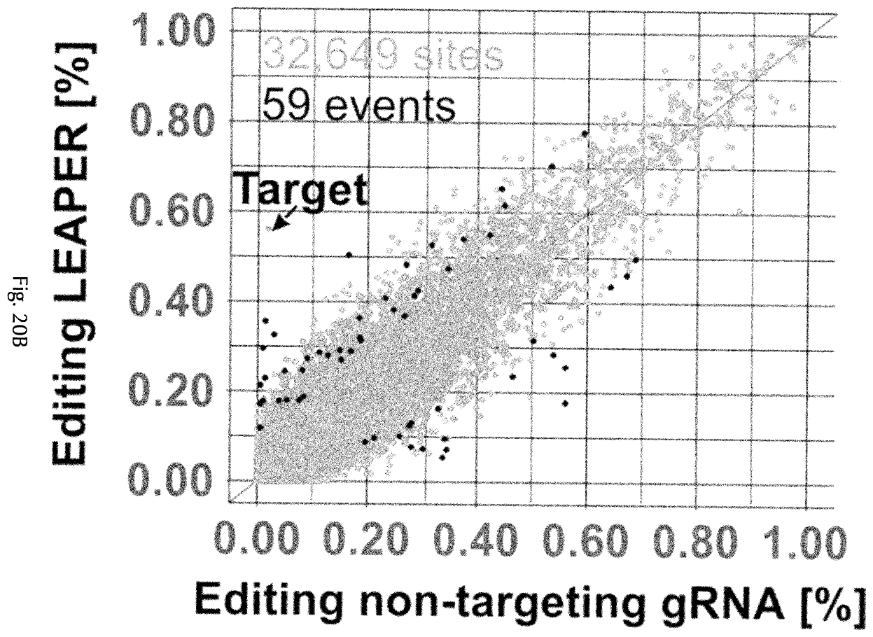

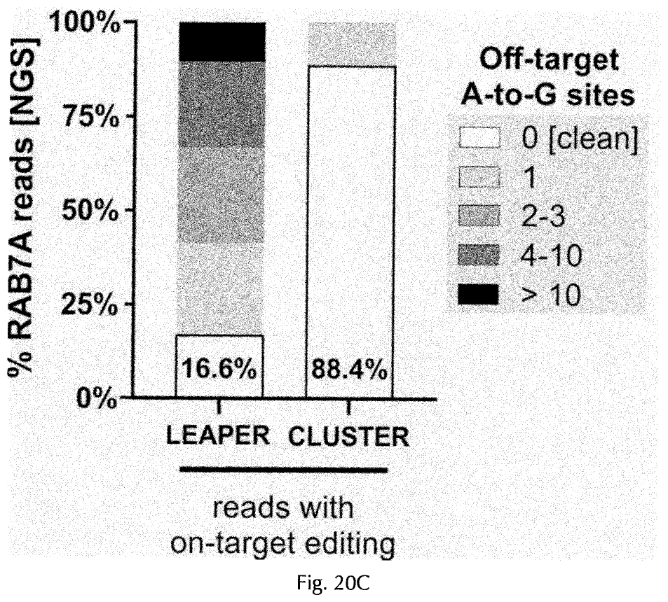

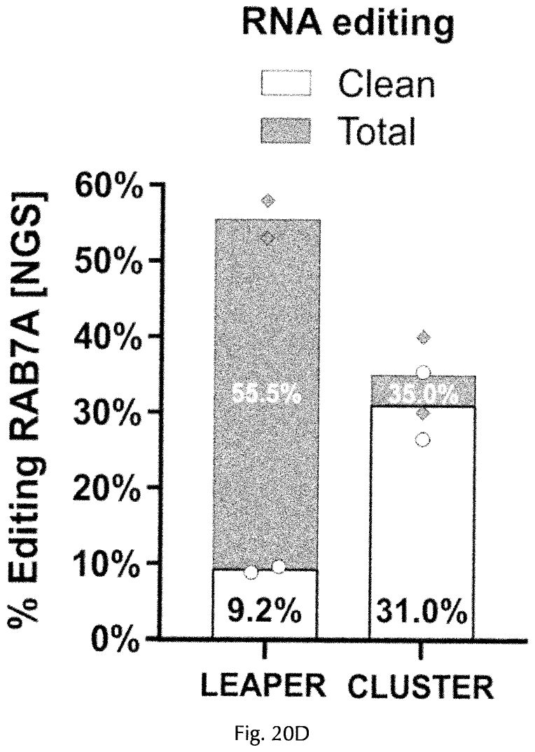

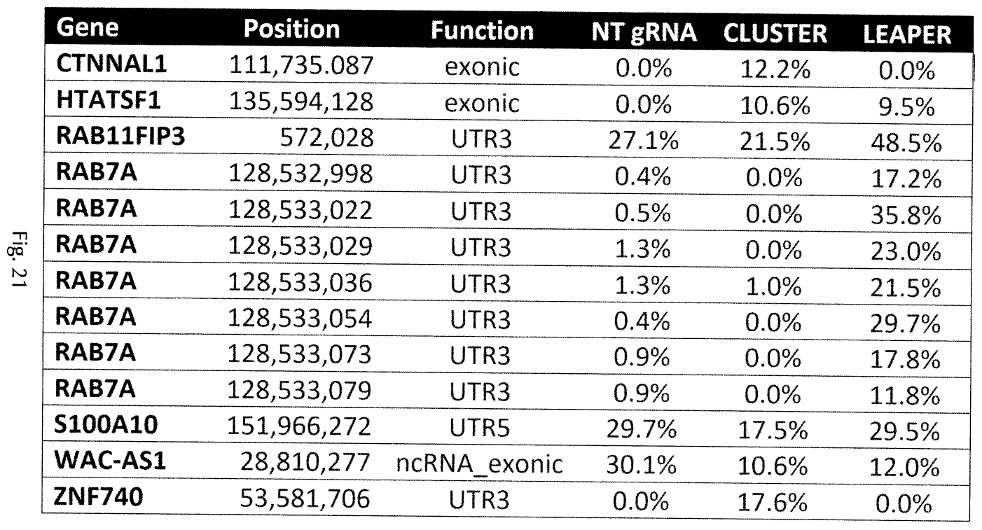

As shown in FIG. 7, the number of RS elements in the invention is important to achieve efficient editing. At least two RS provide for notable editing. A number of 3-8 RS seems optimal. The mere increase of the number of RS, e.g. 20×RS, did not give improved editing yields, however.

### Example 8: Effect of the Length of the Recruitment Sequences on the Editing Yield

In order to investigate the effect of the length of the recruitment sequences on the editing yield, several artificial nucleic acids (guideRNAs) according to the present invention were constructed having a recruiting moiety containing three recruitment sequences of different lengths. The length of the RS and the location of their binding regions on the target mRNA are shown for the different constructs in FIG. 8. The targeted sequence, the binding regions of the RS and the targeted codon (5′-UAG) are indicated. The guideRNAs further contained a 5′-terminal R/G-motif (V21, not shown).

25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

As shown in FIG. 8, the length of the recruitment sequences (RS) is an important optimization parameter in the present invention to achieve efficient editing. Typically, notable editing yields are obtained with recruitment sequences (RS) of 11 nt or above.

### Example 9: Effect of the Linker Length on the Editing Yield

In order to investigate the effect of the length of the nucleotide linker/spacer separating the recruitment sequences on the editing yield, several artificial nucleic acids (guideRNAs) were constructed having linkers and a spacer comprising 0, 1, 2, 3, 4, 5, and 10 adenosine nucleotides, respectively. The upper left part of FIG. 9A describes the representation used to display the interaction of gRNA and mRNA. The hachures and patterns indicated the substructures of the gRNA (targeting sequence, spacer, first recruitment sequence, linker, second recruitment sequence, linker, third recruitment sequence) and the target mRNA (target sequence, distance to next binding region, binding region #1, distance to next binding region, binding region #2, distance to next binding region, binding region #3) as displayed in the legend in the upper right of FIG. 9A. The recruitment sequences that are connected to each other via the linkers and the targeting sequence that is connected to the first recruitment sequences via the spacer bind to the same binding regions on the mRNA for all artificial nucleic acids compared in this example, as displayed in the lower part of FIG. 9A. The FIG. 9B shows the differences between the gRNAs, which differ in the number of adenosine nucleotides between the recruitment sequences.

The editing yield (displayed as relative fold change with respect to the design with 3 adenosine nucleotides as linker) is displayed in FIGS. 9A and 9B, next to the corresponding gRNA binding sites (FIG. 9A) or the gRNA composition, respectively (FIG. 9B). 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

As shown in FIG. 9A (Binding regions of the gRNA on the mRNA) and FIG. 9B (Composition of the guideRNAs), the linker length has a relatively slight effect on the editing yield in the present invention. However, an optimal linker length of three adenosines between RS was observed.

### Example 10: Optimization of the Placement of Three Recruitment Sequences on a Target mRNA

In order to evaluate the effect of the distance separating the regions on the target mRNA where the individual recruitment sequences bind to, several artificial nucleic acids (guideRNAs) were constructed having three 15 nucleotide recruitment sequences which bind to specific regions on the target RNA either with short distance (15-62 nucleotides) or long distance (375-460 nucleotides), respectively. The constructs and their positioning on the target mRNA are shown in FIG. 10.

25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

As shown in FIG. 10, the positioning of the individual RS has an effect on the RNA editing yield, preferring a positioning close to the target site. However, unless one RS is positioned close to the target site, other RS can bind to rather distant regions without severe loss in efficiency, highlighting the flexibility of the guideRNA design in this invention and the large sequence space available for optimization.

### Example 11: Optimization of the Placement of an Extended RS (20 nt) in the Context of Two Short RS (15 nt) on a Target mRNA

In order to evaluate the correlation of the distance of recruitment sequence binding regions on the target mRNA and length of the recruitment sequences, several artificial nucleic acids (guideRNAs) were constructed having two RS of 15 nt and one of 20 nt, respectively, which bind to specific regions on the target RNA with short distance (15-62 nucleotides) or long distance (375-460 nucleotides), respectively. The constructs and their positioning on the target mRNA are shown in FIG. 11.

25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

As shown in FIG. 11, positioning of the RS in close proximity to the target site (short) is preferred over the other setting (long). This is in accordance with example 10, FIG. 10. However, the placement of the extended RS (20 nt) is most effective at the 3′terminal end of the guideRNA in both settings (long and short). Thus, the placement of an extended recruitment sequence (RS) at the 3′-terminal end of the guideRNA might increase the editing yield independent of the RS to RS distance (short versus long). The 3′-terminal RS should therefore preferably be longer than the other RS. The length of RS #1 and #2 seems to be less important.

### Example 12: Effect of Recruitment Sequence Masking on the Editing Yield (Simulation of guideRNAs with Strong Secondary Structure in the Antisense Part)

In order to evaluate the effect of recruitment sequence masking, several artificial nucleic acids (guideRNAs) were constructed each comprising three recruitment sequences (RS #1, RS #2, RS #3) which bind to specific regions on the target mRNA and, to a variable extent, to further recruitment sequences (RS #4, RS #5, RS #6) thereby simulating guideRNAs with strong secondary structure in the antisense part. The constructs are shown in FIG. 12. The upper left part of FIG. 12A describes the representation used to display the interaction of gRNA and mRNA. The hachures and patterns indicated the substructures of the gRNA (targeting sequence, spacer, first recruitment sequence, linker, second recruitment sequence, linker, third recruitment sequence) and the target mRNA (target sequence, distance to next binding region, binding region #1, distance to next binding region, binding region #2, distance to next binding region, binding region #3) as displayed in the legend in the upper right of FIG. 12A. The recruitment sequences and the targeting sequences bind to the same binding regions on the mRNA for all artificial nucleic acids compared in this example, as displayed in the binding schematics part of FIG. 12A. Binding regions whose corresponding recruitment sequence in the gRNA is masked by a masking recruitment sequences (mRS) are highlighted with a vertical line pattern. The masking recruitment sequences (mRS) fold back to the actual mRNA binding recruitment sequences, thereby preventing these recruitment sequences from interacting with their respective binding regions. Binding regions that are not masked by masking recruitment sequences in the gRNA and can thus be used for gRNA-mRNA interaction are highlighted by a right-tilted lines pattern. The gRNA schematics part of FIG. 12A shows the differences between the gRNAs. These differences are the additional masking recruitment sequences (mRS), that fold back to the actual mRNA binding recruitment sequences, thereby masking them for the purpose of gRNA-mRNA interaction.

25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

As shown in FIG. 12A, guideRNAs with masked recruitment sequences show a clearly reduced editing yield relative to the guideRNA comprising three unmasked nucleotide recruitment sequences (3×RS gRNA, no masking). The editing yield decreased step-wise with the increase of number of masked recruitment sequences. Therefore, this experiment suggests that guideRNAs with strong secondary structures in the antisense part (RS and targeting sequence) show a reduced editing yield and should be avoided, which thus has been included into the computational design of the guideRNA sequences.

In FIG. 12B, the minimum free energy (MFE) secondary structures of the guideRNA constructs used in Example 12 are represented. A comparison to the minimum free energy (MFE) structures in FIG. 8B shows how efficiently the described software allows to screen for gRNAs with a weak secondary structure and does also highlight its importance in regard to this experiment.

### Example 13: In-Vivo-Editing of the Dual-Luciferase W417X Amber Reporter in C57BL/6 Mice after HDTV Injection of gRNA and Reporter Plasmids

In order to investigate editing capacity of an artificial nucleic acid of the present invention in an animal experiment, artificial nucleic acid (guideRNA) was delivered into C57BL/6 mice via hydrodynamic tail vein (HDTV) injection.

C57BL/6 mice were treated with endotoxin-free plasmids (NucleoBond® Xtra Midi EF, Macherey Nagel) diluted in saline solution in a total volume equal to 10% of their body-weight. The hydrodynamic tail vein (HDTV) injection of the total volume was performed within 5-10 seconds. Negative control group mice were treated with 10 μg dual-luciferase wt/amb reporter plasmid, the positive control group mice with 10 μg dual-luciferase wt/wt reporter plasmid and the editing group mice with 5 μg dual-luciferase wt/amb reporter plasmid and 25 μg guideRNA plasmid. 72 h after injection the mice were sacrificed. The liver lobes were removed individually. Each lobe was cut into 3 pieces, which were pooled again with the pieces of other lobes to finally get 3 sample categories per mouse, each containing equal amounts of each liver lobe (sample category A, B and C). Samples of category A were used for the luciferase assay, samples of category B for the RNA-isolation and samples of category C as backup material that was stored at −80° C. Samples of category B and C were immediately frozen in liquid nitrogen. Samples of category A were homogenized in 500 μl 1× passive lysis buffer using a micropestle. After spinning the samples down for 5-10 seconds 50 μl sample per well were transferred onto a white 96-well LumiNunc plate (Thermofisher). Each Sample was measured in triplicate in a Tecan Spark 10M plate reader equipped with an auto injector using 35 μl of each assay substrate per well. Samples of category B were homogenized in a 1.5 ml Eppendorf tube using 1 ml TRIzol reagent (Thermofisher) and a micropestle. After adding 200 μl chloroform and vortexing for 30 seconds they were incubated at room temperature for 5-10 minutes. This was followed by centrifugation at 4° C. and 12.000 g for 20 minutes. The aqueous phase was then transferred into a fresh tube and 700 μl ice-cold isopropanol were added. After precipitation over night at −20° C. the precipitate was centrifuged for 60 minutes at 14.000 rpm and washed two times with 500 μl 75% EtOH. After centrifugation at 14.000 rpm for 5 minutes the pellet was dried at 50° C. for 3 minutes and then dissolved in 87.5 μl nuclease-free water. After TRIzol isolation the RNA was DNase-I digested for 30 minutes at 37° C. by adding 10 μl DNase-I buffer and 2.5 μl DNase-I (NEB). Then the RNA was cleaned up a second time this time using the RNeasy Mini RNA isolation kit (QIAGEN). The reverse transcription was performed using ProtoScript II reverse transcriptase (NEB), random primer mix (High-Capacity cDNA Reverse Transcription Kits, Applied Biosystems) and 1 μg of total RNA. After PCR clean-up (NucleoSpin Gel and PCR Clean-up kit, Macherey Nagel) 2.5 μl of the cDNA were used for the Q5-polymerase (NEB) PCRs using the primer pair 2898+2899, followed by a nested PCR using the primer pair 3850+3851. Sanger sequencing (MWG Eurofins Genomics) was performed using primer 3850.

As shown in FIG. 13, in mice treated with 5 μg dual-luciferase wt/amb reporter plasmid and 25 μg guideRNA plasmid luciferase activity was restored to 10.6% compared to the positive control group, as indicated by the restoration of the luciferase signal (protein level). Compared to the negative control group, the luciferase activity of the “editing group” treated with the artificial nucleic acid (guideRNA) of the present invention was increased by a factor of about >1000. The restoration of luciferase signal (10.6% of the positive control) was confirmed at the RNA level by Sanger sequencing after RT-PCR (10.6% editing yield). A representative Sanger sequencing trace and editing yield is presented.

### Example 14: Editing of Endogenous Untranslated Region (UTR) Targets Using Endogenous ADAR1 in HEK293FT Cells

Ras-related protein Rab-7a is a protein that in humans is encoded by the RAB7A gene, and mutations in the RAB7A gene are associated with several diseases including e.g. Charcot-Marie-Tooth Disease.

To evaluate editing of an UTR target sequence of the RAB7A gene, HEK293FT cells were transfected with an artificial nucleic acid (guideRNA) of the present invention.

60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR cleanup and Sanger Sequencing (MWG).

As shown in FIG. 14, the guideRNA of the present invention provides on average editing yields of 20-44%, with the best editing at the RAB7A UTR target site in HEK293FT cells. Notably, there was no bystander editing detectable surrounding the target sites. As an example, the Sanger sequencing trace is given for the RAB7A target site (see FIG. 14, lower panel). A further analysis of the RS binding regions of this target is given in FIG. 16 (LEAPER benchmark).

### Example 15: Editing of Several Endogenous Open Reading Frame (ORF) Targets Using Endogenous ADAR1 in HEK293FT Cells

The NUP43 gene codes for nucleoporin 43, a component of a nuclear pore complex effecting the bidirectional transport of macromolecules between the cytoplasm and the nucleus, and mutations in the NUP43 gene are associated with several diseases including Fanconi anemia, complementation group L, and familiar atrial fibrillation.

GusB is a gene coding for beta-glucoronidase, which is involved in the breakdown of glucosaminoglycans, and mutations in the GusB gene are associated with several diseases including Mucopolysaccharidosis, type VII, and Mucopolysaccharidosis, type VI.

To evaluate editing of an ORF target sequence of the NUP43 and GusB genes, HEK293FT cells were transfected with an artificial nucleic acid (guideRNA) of the present invention.

60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR cleanup and Sanger Sequencing (MWG).

As shown in FIG. 15, the guideRNA of the present invention provides editing yields up to 38%, e.g. when targeting the ORF site L456L in GUSB in transfected HEK293FT cells. Notably, bystander off-target editing is nearly absent surrounding the target sites. Exemplary Sanger sequencing traces around the target site are given for GUSB L456L and the NUP43 V233V (FIG. 15, lower panel). A further analysis of the RS binding regions of the NUP43 target is given in FIG. 16 (LEAPER benchmark).

### Example 16: Benchmark Comparing the On-Target Yield and Unwanted Bystander Off-Target Editing of the Present Invention and Prior Art LEAPER gRNAs

60.000 HEK293FT cells were seeded in 24-well scale in 500 μl DMEM+10% FBS. After 24 h they were transfected with 1200 ng guideRNA plasmid (transfection grade) using a 1:3 ratio of FuGene6. 48 h after transfection the cells were harvested, followed by One-Step RT-PCR using the Biotechrabbit Kit. Directly before the RT-PCR the samples were mixed with 1 μl 10 μM sense-oligo and heated to 70° C. to detach remaining guideRNAs from the target mRNA. Then the RT-PCR mix was added and the PCR performed. This was followed by a 1.4% agarose-gel, PCR cleanup and Sanger Sequencing (MWG).

As shown in FIGS. 16A and 16B, the guideRNAs of the present invention allow efficient on-target editing with high yield while avoiding unwanted bystander off-target editing. The data show that the in-silico selection process of the RS results in massive reduction or even complete avoidance of bystander editing whereas 111 nt LEAPER guideRNAs lack the necessary design flexibility to avoid bystander editing reliably. The LEAPER guideRNA was designed according to Qu et al. (Nature Biotech, supra) as an unstructured 111 nt guideRNA symmetrically positioned around the target site. In accordance to their prior work, the LEAPER guideRNA induced massive bystander off-target editing up to 50% editing yield at various sites, e.g. up to 20 sites in RAB7A (see FIG. 16B). Following their prior work, we included A:G mismatches to suppress bystander editing. However, in one case (NUP43) this was not successful to suppress bystander editing entirely (see FIG. 16A). In the other case (RAB7A) bystander editing could be suppressed but on cost of on-target editing (see FIG. 16B). In contrast, the guideRNAs designed in accordance with the present invention, enabled significantly improved editing specificity (almost no bystander off-target editing) at comparable on-target editing yield.

### Example 17: Generation of a Sequence of an Artificial Nucleic Acid for Site-Directed Editing of a Target RNA Using In-Silico Optimization

In order to facilitate the generation of the sequence of the recruiting moiety of the artificial nucleic acid (guideRNA) comprising at least one recruitment sequence and preferably comprising a cluster of recruitment sequences, which bind to first and further regions of a specific target mRNA to be edited, an in-silico approach is preferably used to detect sequences on the target mRNA suitable as binding sites of the recruitment sequences, and to optimize the artificial nucleic acid (guideRNA), e.g. with respect to potential intramolecular base pairing events. The steps of an exemplary recruitment cluster in-silico optimization are shown in FIGS. 17A to 17C.

Prior to the computer implemented recruitment cluster in-silico optimization, a number of presets are made by a user. In particular, the presets input by the user are made in terms of:
the sequence of the target RNA to be edited;
the length of the targeting sequence (TS) which binds the target sequence on the target RNA (e.g. 16-40 nucleotides);
the length of a recruitment sequence (e.g. 11-16 nucleotides);
the distance, on the target RNA, between the target sequence and the sequence which is bound by the first recruitment sequence (e.g. 10-100 nucleotides);
the distance, on the target RNA, between regions (binding sites) which are bound by the first and further recruitment sequence(s) (e.g. 10-100 nucleotides);
the length of the nucleotide linker (e.g. AAA);
the sequence of the second recruiting moiety (e.g. R/G motif):
an optional 3′ terminal sequence (e.g. UUU).

Based on the above presets input by a user, candidates for guideRNAs suitable for site-directed editing of the input target RNA are created in a “Recruitment cluster in-silico optimization” process.

FIGS. 17A to 17C do not describe the exact combinatorial implementation which is used by the algorithm to process input data, but explain the idea behind the tool in a conceptual way for better understanding.

As shown in FIGS. 17A-17C, in step 1 of the recruitment cluster in-silico optimization method the input cDNA corresponding to the target mRNA is screened for recruitment sequence binding region candidates that only contains G, C, T and GA (there will be no editing when a guanosine nucleotide is located 5′ to the adenosine nucleotide in the target mRNA). For example, the screening is performed 5′ of the target sequence comprising the nucleotide to be edited. In step 2, starting from the target sequence, 5′ located recruitment sequence binding regions which have a specified distance from the target sequence (e.g. 10 to 100 nucleotides) are detected. In step 3, these recruitment sequence binding regions are selected and analysed for their sizes (e.g. 11 and 13 nucleotides). In step 4, the recruitment sequence size input (e.g. 11 nucleotides) is used to generate derivatives of the recruitment sequence binding regions. For example, in case that an uninterrupted recruitment sequence binding region has a length of 13 nucleotides, three derivative recruitment sequences can be created if the input recruitment sequence size was set to 11 nucleotides. In step 5, starting from the first set of derivative recruitment sequence binding regions (1 A, 2A, 2B, 2C), the next recruitment sequence binding regions in the set range (e.g. 10-100 nucleotides) are detected. In step 6, the detected recruitment sequence binding regions are selected and analysed for their sizes. Steps 4 to 6 are repeated until n (e.g. 3) recruitment sequence binding regions are selected. In step 7, the resulting list of recruitment sequence binding regions which all are matching the input variables are converted into recruitment sequences, and the target sequence is converted into the targeting sequence, and the generated sequences are assembled with the input R/G motif (second recruiting moiety), triple adenosine linkers/spacer and three terminal uridines which result from the U6 termination sequence. In step 8, the Vienna RNA package (a set of standalone programs and libraries used for the prediction and analysis of RNA secondary structures) is used to fold all artificial nucleic acids (guideRNAs) within the list and to generate dot-bracket representations of these folds. The ‘Recruitment Cluster Finder’ (RCF) allows sorting the structures by their free energy or by their dot-bracket ratio (ratio of the dot-bracket notation). Structures having a favourable dot-bracket ratio (minimal base pairing within the antisense part of the guideRNA) are further sorted by the lowest number of brackets in a row within the antisense part. The shortest ones, which represent the weakest secondary structures, get the highest ranking. The selected recruitment sequences of resulting guideRNAs with favourable secondary structures can then be blasted to exclude recruitment sequences which might cause off-target editing effects within the transcriptome.

### Example 18: The Recruitment Sequences (RS) and the Double Stranded ADAR Recruiting Domains (e.g. R/G Motif) can be Arranged Around the Targeting Sequence (TS) with Certain Flexibility

Luciferase assay settings of this experiment: 25.000 HeLa cells were seeded in 96-well scale. 24 h post seeding the cells were transfected with 160 ng guideRNA plasmid and 40 ng dual-luciferase reporter per well using a plasmid to Lipofectamine-3000 ratio of 1:1.5. The luciferase assay was performed 48 h post transfection using the Promega dual-luciferase reporter assay system.

As shown in FIGS. 18A-C, it is possible to position recruitment sequences 5′ and/or 3′ of the targeting sequence. While the targeting sequence typically worked very well when placed next to the ADAR recruitment domain, also other placements are possible without losing editing efficiency.

### Example 19: Correction of the Disease Relevant hIDUA W402X Amber Mutation in Primary Fibroblasts from a Hurler Syndrome Patient (Hurler FB, GM06214) Via Transfection of a Chemically Synthesized CLUSTER Antisense Oligonucleotide (ASO)

Fibroblasts from patients with Scheie syndrome (GM01323) and Hurler syndrome (GM06214) were purchased from the Coriell Institute for Medical Research (USA). 2.5×10⁵ cells/well in 2.5 ml DMEM+15% FBS were seeded into 6-well plates. For each tested condition, two 6-wells were used for the IDUA assay and one 6-well was used to determine RNA editing yields by Sanger sequencing. The CLUSTER ASO was a PAGE-purified, endblocked (2′-OMe, PS) RNA oligonucleotide with a 3×RS (20-20p8-25-20) CLUSTER design, which was ligated (T4 RNA ligase) inhouse from two commercially purchased (Biospring GmbH, Germany) and HPLC purified oligonucleotides of 69 nt (5′part) and 80 nt (3′part) length. The full sequence and modification pattern is given in the list of applied gRNAs below. Transfection was performed 24 h after seeding with 125 pmol ASO and 7.5 μl RNAiMAX, each diluted in 250 μl Opti-MEM. Both solutions were combined after 5 min incubation and incubated for an additional 20 min before the transfection mix was distributed evenly into one well. The medium was changed 24 h after transfection.

For Sanger sequencing: 48 h after transfection, fibroblasts were harvested in RLT-buffer (QIAGEN, #79216), followed by RNA isolation using the Monarch RNA clean up kit (NEB, (#T2030L). Turbo-DNase digestion was performed using the TURBO DNA-free Kit (Thermofisher, #AM1907). Reverse transcription was performed using the SuperScript IV RT (Thermofisher, #18090050) with random primers for 1 h at 52° C. A regular PCR followed by a nested PCR was performed using Taq Polymerase (NEB, #M0267S) with each reaction containing 10% DMSO. After the second PCR the products were separated by SB-agarose gel-electrophoresis. After PCR cleanup (NucleoSpin Gel and PCR Clean-up Kit, Macherey Nagel, #740609), Sanger sequencing (Microsynth AG) was performed.

For the α-L-iduronidase enzyme activity assay: The medium was changed 24 h after transfection. 48 h after transfection, fibroblasts were detached and washed once with PBS. 40 μl 0.5% Triton X-100 in PBS were added to the cell pellet and incubated on ice for 30 min and α-L-iduronidase enzyme assay was performed. For the editing read-out via α-L-iduronidase enzyme activity assay a standard dilution series of 4-methylumbelliferone (Sigma Aldrich, M1381) was prepared in 1× PBS. For each concentration, 25 μl of the standard solution were added to 25 μl 0.4 M sodium formate buffer (pH 3.5) and applied to a 96-well LumiNunc plate (VWR, 732-2696) in triplicate. The substrate (4-methylumbelliferyl α-L-iduronide, Glycosynth, #44076) was dissolved in 0.4 M sodium formate buffer to a final concentration of 180 μM. For the murine IDUA assay using HeLa cells 25 μl of the 1:3 diluted cell lysate (0.5% Tween-20/PBS) was added to 25 μl substrate in the plate and incubated for 45 min at 37° C. in the dark. 25 μl of undiluted cell lysate (0.5% Triton X-100/PBS) was added to 25 μl substrate in the plate and incubated for 90 min at 37° C. in the dark. The reaction was quenched in both cases by adding 200 μl glycine carbonate buffer (0.17 M Glycine/NaOH, pH 10.4). The fluorescence of 4-methylumbelliferone was measured with an excitation wavelength of 355 nm at an emission wavelength of 460 nm with a Tecan Spark 10M plate reader. Calculated enzyme activities were referenced to the protein amount as determined by BCA assay (Pierce BCA Protein Assay Kit, Thermofisher, 23227). The enzyme activity was standardized to Scheie fibroblast lysate.

In these experiments, the CLUSTER approach for the restoration of hIDUA activity in fibroblasts taken from a Hurler patient was tested. To overcome the strong plasmid transfection bias in these cells, the CLUSTER guideRNA in form of an antisense oligonucleotide was applied. With Sanger sequencing guideRNA-dependent editing yields of 24% were determined (FIG. 19A) and a restoration of IDUA enzyme activity to the levels obtained with Scheie control fibroblasts was observed (FIG. 19B). As Scheie syndrome is a much less severe disease, the data indicates that clinically beneficial effects might be within reach.

### Example 20: NGS Characterization of Global Off-Target Effects and Quantification of Clean Editing Events for CLUSTER and LEAPER gRNAs

The RNA editing experiment was performed by transfection of 1200 ng guide RNA plasmid (NucleoSpin Plasmid Transfection-grade, Macherey Nagel, #740490) into 6×10⁴ HEK293FT cells 24 h post seeding using a 1:3 ratio of FuGene6 (Promega, #E2691) in 24 cell format. 48 h after transfection, cells were harvested. Overall, three settings were carried out, each with an independent duplicate. Those settings include a non-targeting guide RNA (NT-RNA) and a RAB7A 3′UTR 19-11-13-20p8 CLUSTER guide RNA. RNA was isolated with the RNeasy MinElute Kit (Qiagen, #74204), treated with DNase I (NEB, #M0303S), incubated with an RNA strand reverse complementary to the antisense part of the respective guide RNA, heated to 95° C. for 3 min and purified again with the RNeasy MinElute Kit. Purified RNA was delivered to CeGaT (Germany) for poly(A)+mRNA sequencing. The library was prepared from 200 ng RNA with the TruSeq Stranded mRNA Library Prep Kit (Illumina, USA) and sequenced with a NovaSeq 6000 (50M reads, 2×100 bp paired end, Illumina, USA). Mapping of RNA-seq and reads: A previously published pipeline to accurately align RNA-seq reads onto the genome was adopted (see Ramaswami, G., et al., Accurate identification of human Alu and non-Alu RNA editing sites. Nat Methods, 2012. 9(6): p. 579-81; Ramaswami, G., et al., Identifying RNA editing sites using RNA sequencing data alone. Nat Methods, 2013. 10(2): p. 128-32). A STAR (version 2.5.3a) (see Dobin, A., et al., STAR: ultrafast universal RNA-seq aligner. Bioinformatics, 2013. 29(1): p. 15-21) was used to align the reads to the hg19 reference genome and ran Picard tools (version 1.129) to remove clonal reads (PCR duplicates) mapped to the same location. Of these identical reads, only the read with the highest mapping quality was kept for downstream analysis. Unique and nonduplicate reads were subjected to local realignment and base score recalibration using the IndelRealigner and TableRecalibration from the Genome Analysis Toolkit (GATK, version 3.6) (see Li, H., et al., The Sequence Alignment/Map format and SAMtools. Bioinformatics, 2009. 25(16): p. 2078-9). The above steps were applied separately to each of the RNA-seq samples. Identification of editing sites from RNA-seq data: To remove LEAPER guide RNA sequences that were falsely aligned to the targeting region, the rmdup command in samtools (see Li, H., et al., The Sequence Alignment/Map format and SAMtools. Bioinformatics, 2009. 25(16): p. 2078-9) was used to remove PCR duplicates in the RAB7A 3′UTR region. Additionally, all reads that contained the sequence ‘AAGGGTG’ (3′end of the LEAPER gRNA) and reads that ended with ‘TCAAAGAC’ (5′-end of the LEAPER gRNA) were removed. As last step, all reads that originated from the antisense sequence of the RAB7A gene were removed. This procedure was applied to all samples (CLUSTER, LEAPER, non-targeting gRNA). To call variants from the mapped RNA-seq reads, the UnifiedGenotyper from GATK (see McKenna, A., et al., The Genome Analysis Toolkit: a MapReduce framework for analyzing next-generation DNA sequencing data. Genome Res, 2010. 20(9): p. 1297-303) was used. In contrast to the usual practice of variant calling, the variants with relatively loose criteria were identified by using the UnifiedGenotyper tool with options stand_call_conf 0, stand_emit_conf 0, and output mode EMIT_VARIANTS_ONLY. Variants from nonrepetitive and repetitive non-Alu regions were required to be supported by at least three reads containing mismatches between the reference genome sequences and RNA-seq. Supporting of one mismatch read was required for variants in Alu regions. This set of variant candidates was subject to several filtering steps to increase the accuracy of editing site calling. First all known human SNPs present in dbSNP build 137 were removed (except SNPs of molecular type “cDNA”; database version 135; http://www.ncbi.nlm.nih.gov/SNP/), the 1000 Genomes Project, and the University of Washington Exome Sequencing Project (http://evs.gs.washington.edu/EVS/). To remove false-positive RNA-seq variant calls due to technical artefacts, further filters were applied as previously described (see Ramaswami, G., et al., Accurate identification of human Alu and non-Alu RNA editing sites. Nat Methods, 2012. 9(6): p. 579-81; Merkle, T. and T. Stafforst, New Frontiers for Site-Directed RNA Editing: Harnessing Endogenous ADARs. Methods Mol Biol, 2021. 2181: p. 331-349). In brief, requirung a variant call quality Q>20 (see Ramaswami, G., et al., Accurate identification of human Alu and non-Alu RNA editing sites. Nat Methods, 2012. 9(6): p. 579-81; Merkle, T. and T. Stafforst, New Frontiers for Site-Directed RNA Editing: Harnessing Endogenous ADARs. Methods Mol Biol, 2021. 2181: p. 331-349), variants were discarded if they occurred in the first 6 bases of a read, variants in simple repeats were removed (see Li, H., et al., The Sequence Alignment/Map format and SAMtools. Bioinformatics, 2009. 25(16): p. 2078-9), intronic variants that were within 4 bp of splice junctions were removed, and variants in homopolymers were discarded. Moreover, reads mapped to highly similar regions of the transcriptome by BLAT were removed (see Kent, W. J., BLAT—the BLAST-like alignment tool. Genome Res, 2002. 12(4): p. 656-64). Finally, variants were annotated using ANNOVAR (see Wang, K., M. Li, and H. Hakonarson, ANNOVAR: functional annotation of genetic variants from high-throughput sequencing data. Nucleic Acids Res, 2010. 38(16): p. e164) based on gene models from Gencode, RefSeq, Ensembl and UCSC. All sites identified from RNA-seq data were compared with all sites available in the RADAR database (see Ramaswami, G. and J. B. Li, RADAR: a rigorously annotated database of A-to-I RNA editing. Nucleic Acids Res, 2014. 42(Database issue): p. D109-13) to be referred as ‘known’ sites if found in RADAR or ‘novel’ sites if not found. Identification of significantly differently edited sites: All sites found in the RNA seq samples with the sites of the RADAR database were merged, and editing levels of edited sites with 50 reads coverage (combined coverage of both replicates) were quantified, and Fisher's exact tests followed by Benjamini-Hochberg's multiple test correction (adjusted P<0.01) were performed to identify significantly differently edited sites across the samples (absolute editing difference >10%). Measuring RAB7A editing precision (clean reads): To compare the specificity of CLUSTER and LEAPER guide RNA, all mapped reads that contained the edited target sequence ‘GCTGGCGG’ were selected. The edited reads and their partner reads were compared to the RAB7A sequence covering the editing region to identify A-to-G mismatches. As a control, the non-targeting sample was used to quantify A-to-G mismatches in the reads that covered the unedited target sequence ‘GCTAGCGG’.

To compare global off-target effects and low-level bystander editing between the LEAPER and the CLUSTER approach, a transcriptome-wide Poly(A)+RNA sequencing experiment for the RAB7A target in HEK293FT cells was performed. A non-targeting guide RNA was transfected as a control. An established pipeline (see Merkle, T., et al., Precise RNA editing by recruiting endogenous ADARs with antisense oligonucleotides. Nat Biotechnol, 2019. 37(2): p. 133-138) was used, to identify significantly differently edited sites in the transcriptome, and a small number of hits for the LEAPER (59) and the CLUSTER (44) guide RNA (FIGS. 20A and 20B), respectively, was found. This number of global off-target events is quite low, compared to systems that utilizes engineered editases (e.g. Lambda-N-ADAR, Cas13-ADAR), where thousands of events were reported. The NGS approach does also allow to study the frequency by which on-target editing is corrupted with bystander editing within the same reads. While 88.4% of the reads with the CLUSTER guide RNA were cleanly edited, this was only the case for 16.6% of the reads in the LEAPER guide RNA (FIG. 20C). Factoring in this, the total yield of clean on-target editing was 31% for the CLUSTER guide RNA clearly outcompeting the LEAPER guide RNA, which achieved only 9.2% (FIG. 20D).

### Example 21: List of Off-Target Events Induced by RNA Editing with CLUSTER- and LEAPER-Guide RNAs Determined by Next Generation Sequencing

The RNA editing experiment and the NGS data processing was performed as explained for example 20. The list shows the subset of off-target editing events at “unknown” sites that have been detected by the pipeline searching for significantly differentially edited sites in at least one sample when cells were treated either with the CLUSTER or the LEAPER guide RNA and compared to the non-targeting control guide RNA (NT gRNA). Sites were assigned “unknown” when they were not listed in the RADAR database. Nonsynonymous editing was only detected for a single site, HTATSF1 (S742G). Mapping analysis detected sites for potential off-target binding of the RAB7A guide RNAs to CTNNAL1, HTATSF1, and ZNF740. The detected RAB7A sites represent the fraction of bystander editing sites close to the on-target site that met the significance and cut-off criteria of the pipeline. Data are representing the mean±s.d. of N=2 NGS replicates.

The number of sites, which are novel and have not been edited in the non-targeting control were only 3 for the CLUSTER guide RNA (beside the target site) and 7 for the LEAPER guide RNA (FIG. 21). For the CLUSTER guide RNA, two of the three novel sites were exonic sites (HTATSF1, CTNNAL1). Both contain putative binding sites for the guide RNA and gave low editing yields (12.2%). The HTATSF1 editing site was the only one that gives a nonsynonymous amino acid change. Notably, the LEAPER guide RNA edited that site to a similar extent. The other seven detected novel sites with the LEAPER guide RNA were bystander off-target editings in close proximity to the on-target site.

List of applied guideRNAs (for the ease of use, the guideRNAs are given as coding DNA sequences):

| | | | Used |
|---|---|---|---|
| | | | in |
| pTS # | GuideRNA Name | Sequence 5′→3′ | Figure |
| pTS967 | Luciferase_W417X_RG- | Ggtgtcgagaagaggagaacaatatgctaaatgttgttctcgtctcctcgacac | #1, #7 |
| | V21_0xRS_20p8 | cgtgcagccagccgtccttgt | |
| pTS961 | Luciferase_W417X_RG- | Ggtgtcgagaagaggagaacaatatgctaaatgttgttctcgtctcctcgacac | #1, #2, |
| | V21_3xRS(15-13-11- | cgtgcagccagccgtccttgtaaacgcacagctcgaaagtccaagtccacca | #3, #4, |
| | 20p8)_3xA | aaaagaagggcaccacc | #6, #7, |
| | | | #8, #9, |
| | | | #13 |
| pTS970 | Luciferase W417X RG- | ggtgtcgagaagaggagaacaatatgctaaatgttgttctcgtctcctcgacac | #1, #6, |
| | V21_8xRS(11-15-15- | cgtgcagccagccgtccttgtaaacgcacagctcgaaagtccaagtccacca | #7 |
| | 15-12-15-13-11- | aaaagaagggcaccaccaaactcctcctcgaaaaaaaagccgcagatcaa | |
| | 20p8)_3xA | aaacgaagctctcgaaaccacagccacaccgaaaacacaccacgatccga | |
| pTS558 | Luciferase_W417X_RG- | gtggtcgagaagaggagaacaatatgctaaatgttgttctcgtctcctcgacca | #2 |
| | V20_0xRS_16p8_BoxB | cgtgcagccagccgtcctctagagggccctgaagagggccc | |
| pTS1129 | AHI_W725X_RG- | Ggtgtcgagaagaggagaacaatatgctaaatgttgttctcgtctcctcgacac | #5 |
| | V21_9xRS_20p8 | caactttccatatccgtatcaaaaaaacaaaagaaggaaaaaaacgtccaga | |
| | | agaaagagaaatcaagaaaagaagagccatcaaaacctcaacaacacaaa | |
| | | aagaaatcaagaataaacctcgaagcaaaaaagaaacaggccgtccacaa | |
| | | aggtcatcatcaagcaa | |
| pTS1130 | BMPR2_W298X_RG- | Ggtgtcgagaagaggagaacaatatgctaaatgttgttctcgtctcctcgacac | #5 |
| | V21_9xRS_20p8 | cgcttacccagtcacttgtgtaaaactccatcacaaaaactctctcatctccaa | |
| | | caaatccatcaaaggcactcaaagcaaaggaaaacacaaagacgctcatc | |
| | | caagaaaacctcggccaatcagaaaatcaaaacagcaaaaacagcagaaa | |
| | | cgaaaagaccaacatcc | |
| pTS1131 | COL3A1_W1278X_RG- | Ggtgtcgagaagaggagaacaatatgctaaatgttgttctcgtctcctcgacac | #5 |
| | V21_9xRS_20p8 | cgtcaacccagtattctccacaaaagaaccatcagaaagggcaaaaccgca | |
| | | aaacctccaatcccagcaaaacaacaccaccacagcaaaaccctcagatc | |
| | | ctcaaaggtccaacaggtcctcaaacagcagctccacgaaaggccagcag | |
| | | ggccacaaacaccacgatca | |
| pTS1132 | FANCC_W506X_RG- | Ggtgtcgagaagaggagaacaatatgctaaatgttgttctcgtctcctcgacac | #5 |
| | V21_9xRS_20p8 | cgacatcccaggcgatcgtgtaaagcctcccatcacaaatctcagcccatcct | |
| | | ccaaagaaccagctctcaaagaaacaaaccgcagcaaatccagggcccca | |
| | | tcgaaactcatcaacaacccggaaagccgaagccagaaaacctcaaagaa | |
| | | ctcaaaggacacaaactc | |
| pTS1136 | MYBPC3_W1098X_RG- | ggtgtcgagaagaggagaacaatatgctaaatgttgttctcgtctcctcgacac | # 5 |
| | V21_9xRS_20p8 | cgtacccccagagctccgtgtaaatccactccagaaaacgtcagtcacccga | |
| | | aagcaccgtcaccaaatcacctcctcgaaatcacaggctccccgacaaaga | |
| | | aaagcagccgaaacagccacccactaaatcccacgcgctaaaagacgcgc | |
| | | atct | |
| pTS1135 | IL2RG_W237X RG- | Gggagtggggctgtttgtgttttgtgttctttcttctggctgttttcggactgac | #5 |
| | V21_6xRS_20p8 | tgggactttgtgtggatgggctttgtttcgtgttcggagctttgtgctcagcatt | |
| | | ggagtgaaggtgtcgaggagacgagaacaacatttagcatattgttctcctcttc | |
| | | tcgacacc | |
| pTS1180 | PINK1_W437X_RG- | Ggtgtcgagaagaggagaacaatatgctaaatgttgttctcgtctcctcgacac | #5 |
| | V21_3xRS_20p8 | ccactgcccaggcatcagcctaaaccgccccgatccacgaaatctcatcag | |
| | | ccagaaaatcaccagcca | |
| pTS1181 | Luciferase_W417X_RG- | ggtgtcgagaagaggagaacaatatgctaaatgttgttctcgtctcctcgacac | #7 |
| | V21_1xRS_20p8 | cgtgcagccagccgtccttgtaaacgcacagctcg | |
| pTS1182 | Luciferase_W417X_RG- | ggtgtcgagaagaggagaacaatatgctaaatgttgttctcgtctcctcgacac | #7 |
| | V21_2xRS_20p8 | cgtgcagccagccgtccttgtaaacgcacagctcgaaagtccaagtccacc | |
| pTS1183 | Luciferase_W417X_RG- | Ggtgtcgagaagaggagaacaatatgctaaatgttgttctcgtctcctcgacac | #7 |
| | V21_6xRS_20p8 | cgtgcagccagccgtccttgtaaacgcacagctcgaaagtccaagtccacca | |
| | | aaaagaagggcaccaccaaactcctcctcgaaaaaaaagccgcagatcaa | |
| | | aaacgaagctctcg | |
| pTS1211 | Luciferase_W417X_RG- | ggtgtcgagaagaggagaacaatatgctaaatgttgttctcgtctcctcgacac | #7 |
| | V21_20xRS_20p8 | cgtgcagccagccgtccttgtaaacgcacagctcgaaagtccaagtccacca | |
| | | aaaagaagggcaccaccaaactcctcctcgaaaaaaaagccgcagatcaa | |
| | | aaacgaagctctcgaaaccacagccacaccgaaaacacaccacgatccga | |
| | | aaagaacgctcatctcaaactcgccggcggtcccaaactcctccacgtctcc | |
| | | aaaatcaacgaacgaaaagaaaagaatccaaaacaacgtcaggaaatctc | |
| | | caaaaccaaaatcaatcacatcaaacaccccaatcaaaaacaccgcgcaa | |
| | | agaatcaagaacaaaagcccaccact | |
| pTS1184 | Luciferase_W417X_RG- | ggtgtcgagaagaggagaacaatatgctaaatgttgttctcgtctcctcgacac | #8 |
| | V21_3xRS(7-7-7-20p8) | cgtgcagccagccgtccttgtaaacctcgggaaaaagtccaaaagaagaag | |
| pTS1185 | Luciferase_W417X RG- | Ggtgtcgagaagaggagaacaatatgctaaatgttgttctcgtctcctcgacac | #8 |
| | V21_3xRS(9-9-9-20p8) | cgtgcagccagccgtccttgtaaaacgcacagcaaacacacccagaaatcc | |
| | | accacc | |
| pTS1186 | Luciferase_W417X_RG- | Ggtgtcgagaagaggagaacaatatgctaaatgttgttctcgtctcctcgacac | #8 |
| | V21_3xRS(11-11-11- | cgtgcagccagccgtccttgtaaacgcacagctcgaaacaagtccaccaaa | |
| | 20p8) | aagggcaccacc | |
| pTS1187 | Luciferase_W417X_RG- | Ggtgtcgagaagaggagaacaatatgctaaatgttgttctcgtctcctcgacac | #8 |
| | V21_3xRS(13-13-13- | cgtgcagccagccgtccttgtaaaacggacgcacagcaaaccaagtccacc | |
| | 20p8) | acaaaagaagggcaccac | |
| pTS1188 | Luciferase_W417X_RG- | Ggtgtcgagaagaggagaacaatatgctaaatgttgttctcgtctcctcgacac | #8, |
| | V21_3xRS(15-S-15-S- | cgtgcagccagccgtccttgtaaaacgcacagctcgccgaaagtccaagtcc | #10, |
| | 15-S-20p8) | accacaaaaagaagggcaccacc | #11, |
| | | | #18 |
| pTS1297 | Luciferase_W417X_RG- | Ggtgtcgagaagaggagaacaatatgctaaatgttgttctcgtctcctcgacac | #9 |
| | V21_3xRS(15-13-11- | cgtgcagccagccgtccttgtcgcacagctcggtccaagtccaccaagaagg | |
| | 20p8)_0xA | gcaccacc | |
| pTS1173 | Luciferase_W417X_RG- | Ggtgtcgagaagaggagaacaatatgctaaatgttgttctcgtctcctcgacac | #9 |
| | V21_3xRS(15-13-11- | cgtgcagccagccgtccttgtacgcacagctcgagtccaagtccaccaaaga | |
| | 20p8)_1xA | agggcaccacc | |
| pTS1174 | Luciferase_W417X_RG- | Ggtgtcgagaagaggagaacaatatgctaaatgttgttctcgtctcctcgacac | #9 |
| | V21_3xRS(15-13-11- | cgtgcagccagccgtccttgtaacgcacagctcgaagtccaagtccaccaaa | |
| | 20p8)_2xA | agaagggcaccacc | |
| pTS1175 | Luciferase_W417X_RG- | Ggtgtcgagaagaggagaacaatatgctaaatgttgttctcgtctcctcgacac | #9 |
| | V21_3xRS(15-13-11- | cgtgcagccagccgtccttgtaaaacgcacagctcgaaaagtccaagtccac | |
| | 20p8)_4xA | caaaaaagaagggcaccacc | |
| pTS1176 | Luciferase_W417X RG- | Ggtgtcgagaagaggagaacaatatgctaaatgttgttctcgtctcctcgacac | #9 |
| | V21_3xRS(15-13-11- | cgtgcagccagccgtccttgtaaaaacgcacagctcgaaaaagtccaagtcc | |
| | 20p8)_5xA | accaaaaaaagaagggcaccacc | |
| pTS1298 | Luciferase_W417X_RG- | ggtgtcgagaagaggagaacaatatgctaaatgttgttctcgtctcctcgacac | #9 |
| | V21_3xRS(15-13-11- | cgtgcagccagccgtccttgtaaaaaaaaaacgcacagctcgaaaaaaaaa | |
| | 20p8)_10xA | agtccaagtccaccaaaaaaaaaaaagaagggcaccac | |
| pTS1261 | Luciferase_W417X_RG- | Ggtgtcgagaagaggagaacaatatgctaaatgttgttctcgtctcctcgacac | #10 |
| | V21_3xRS(15-L-15-S- | cgtgcagccagccgtccttgtaaaacgcacagctcgccgaaagtccaagtcc | |
| | 15-S-20p8) | accacaaacgaaagccgcagatc | |
| pTS1262 | Luciferase_W417X_RG- | Ggtgtcgagaagaggagaacaatatgctaaatgttgttctcgtctcctcgacac | #10 |
| | V21_3xRS(15-L-15-L- | cgtgcagccagccgtccttgtaaaacgcacagctcgccgaaacgaaagccg | |
| | 15-S-20p8) | cagatcaaaccacagccacaccga | |
| pTS1263 | Luciferase_W417X_RG- | Ggtgtcgagaagaggagaacaatatgctaaatgttgttctcgtctcctcgacac | #10 |
| | V21_3xRS(15-S-15-L- | cgtgcagccagccgtccttgtaaaacgcacagctcgccgaaacgaaagccg | |
| | 15-S-20p8) | cagatcaaagaacatgccgaagcc | |
| pTS1264 | Luciferase_W417X_RG- | Ggtgtcgagaagaggagaacaatatgctaaatgttgttctcgtctcctcgacac | #10 |
| | V21_3xRS(15-S-15-S- | cgtgcagccagccgtccttgtaaacgaaagccgcagatcaaagaacatgcc | |
| | 15-L-20p8) | gaagccaaaatggggtcgcgggca | |
| pTS1265 | Luciferase_W417X_RG- | Ggtgtcgagaagaggagaacaatatgctaaatgttgttctcgtctcctcgacac | #10 |
| | V21_3xRS(15-S-15-L- | cgtgcagccagccgtccttgtaaacgaaagccgcagatcaaaccacagcca | |
| | 15-L-20p8) | caccgaaaacacccaacacgggca | |
| pTS1266 | Luciferase_W417X_RG- | Ggtgtcgagaagaggagaacaatatgctaaatgttgttctcgtctcctcgacac | #10 |
| | V21_3xRS(15-L-15-S- | cgtgcagccagccgtccttgtaaacgaaagccgcagatcaaagaacatgcc | |
| | 15-L-20p8) | gaagccaaacacccaacacgggca | |
| pTS1267 | Luciferase_W417X_RG- | Ggtgtcgagaagaggagaacaatatgctaaatgttgttctcgtctcctcgacac | #10, |
| | V21_3xRS(15-L-15-L- | cgtgcagccagccgtccttgtaaacgaaagccgcagatcaaaccacagcca | #11 |
| | 215-L-0p8) | caccgaaaactcgctcaacgaacg | |
| pTS1258 | Luciferase_W417X_RG- | ggtgtcgagaagaggagaacaatatgctaaatgttgttctcgtctcctcgacac | #11, |
| | V21_3xRS(20-S-15-S- | cgtgcagccagccgtccttgtaaaacgcacagctcgccgaaagtccaagtcc | #14 |
| | 15-S-20p8) | accacaaacctcgaagaagggcaccacc | |
| pTS1259 | Luciferase_W417X RG- | ggtgtcgagaagaggagaacaatatgctaaatgttgttctcgtctcctcgacac | #11 |
| | V21_3xRS(15-S-20-S- | cgtgcagccagccgtccttgtaaaacgcacagctcgccgaaaccggtgtcca | |
| | 15-S-20p8) | agtccaccacaaaaagaagggcaccacc | |
| pTS1260 | Luciferase_W417X_RG- | ggtgtcgagaagaggagaacaatatgctaaatgttgttctcgtctcctcgacac | #11 |
| | V21_3xRS(15-S-15-S- | cgtgcagccagccgtccttgtaaacacggacgcacagctcgccgaaagtcc | |
| | 20-S-20p8) | aagtccaccacaaaaagaagggcaccacc | |
| pTS1277 | Luciferase_W417X_RG- | ggtgtcgagaagaggagaacaatatgctaaatgttgttctcgtctcctcgacac | #11 |
| | V21_3xRS(20-L-15-L- | cgtgcagccagccgtccttgtaaacgaaagccgcagatcaaaccacagcca | |
| | 15-L-20p8) | caccgaaaagagaactcgctcaacgaacg | |
| pTS1278 | Luciferase_W417X_RG- | ggtgtcgagaagaggagaacaatatgctaaatgttgttctcgtctcctcgacac | #11 |
| | V21_3xRS(15-L-20-L- | cgtgcagccagccgtccttgtaaacgaaagccgcagatcaaaggggccaca | |
| | 15-L-20p8) | gccacaccgataaactcgctcaacgaacg | |
| pTS1279 | Luciferase_W417X_RG- | ggtgtcgagaagaggagaacaatatgctaaatgttgttctcgtctcctcgacac | #11 |
| | V21_3xRS(15-L-15-L- | cgtgcagccagccgtccttgtaaacgacccgaaagccgcagatcaaaccac | |
| | 20-L-20p8) | agccacaccgaaaactcgctcaacgaacg | |
| pTS1189 | Luciferase_W417X RG- | Ggtgtcgagaagaggagaacaatatgctaaatgttgttctcgtctcctcgacac | #13 |
| | V21_3xRS(RS#1- | cgtgcagccagccgtccttgtaaacgcacagctcgaaagtccaagtccacca | |
| | 3mask_15- | aaaagaagggcaccaccaaaggtggtgcccttcttaaaggtggacttggaca | |
| | 13-11-20p8) | aacgagctgtgcg | |
| pTS1295 | Luciferase_W417X_RG- | ggtgtcgagaagaggagaacaatatgctaaatgttgttctcgtctcctcgacac | #13 |
| | V21_3xRS(RS#3mask_ | cgtgcagccagccgtccttgtaaacgcacagctcgaaagtccaagtccacca | |
| | 15-13-11-20p8) | aaaagaagggcaccaccaaaggtggtgcccttctt | |
| pTS1296 | Luciferase_W417X_RG- | ggtgtcgagaagaggagaacaatatgctaaatgttgttctcgtctcctcgacac | #13 |
| | V21_3xRS(RS#2 + | cgtgcagccagccgtccttgtaaacgcacagctcgaaagtccaagtccacca | |
| | 3mask_15-13-11-20p8) | aaaagaagggcaccaccaaaggtggtgcccttcttaaaggtggacttggac | |
| pTS1362 | RAB7A_3′UTR_TAG#1_ | ggtgtcgagaagaggagaacaatatgctaaatgttgttctcgtctcctcgacac | #15, |
| | RG-V21_3xRS(20-15- | cctgccgccagctggatttccaaattcaaccctccacctaaatacaaaactca | #17 |
| | 15-20p8)_3xA | gcctaaataaaaagaccacaagaccaa | |
| pTS985 | RAB7A_3′UTR_TAG#1_ | Ggtgtcgagaagaggagaacaatatgctaaatgttgttctcgtctcctcgacac | #15, |
| | RG-V21_3xRS(19-13- | cctgccgccagctggatttccaaatcaaccctccaccaaaacaaaactcaga | #17, |
| | 11-20p8)_3xA | aaaaaaagaccacaagaccaa | #20, |
| | | | #21 |
| pTS1341 | β-Actin_3′UTR_ | ggtgtcgagaagaggagaacaatatgctaaatgttgttctcgtctcctcgacac | #15 |
| | TAG#1_RG-V21_3xRS | cacgcaaccaagtcatagtccaaaggaggggccggactcaaaccgccgat | |
| | (20-15-15-20p8)_3xA | ccacacgaaatgcgctcaggaggagcaatg | |
| pTS1342 | β-Actin_3′UTR_ | ggtgtcgagaagaggagaacaatatgctaaatgttgttctcgtctcctcgacac | #15 |
| | TAG#1_RG-V21_3xRS | cacgcaaccaagtcatagtccaaaagccgccgatccacacggagaaacct | |
| | (20-20-20-20p8)_3xA | cagggcagcggaaccgcaaagctcgaagtccagggcgacg | |
| pTS1344 | GAPDH_3′UTR_TAG#1_ | ggtgtcgagaagaggagaacaatatgctaaatgttgttctcgtctcctcgacac | #15 |
| | RG-V21_3xRS(20-15- | caggggtccacatggcaactgaaacagggactccccagcaaagaagtcag | |
| | 15-20p8)_3xA | aggagacaaatagacggcaggtcaggtcca | |
| pTS1346 | GAPDH_3′UTR TAG#1_ | ggtgtcgagaagaggagaacaatatgctaaatgttgttctcgtctcctcgacac | #15 |
| | RG-V21_3xRS(20-20- | caggggtccacatggcaactgaaatgtggcagggactccccagcaaagaag | |
| | 20-20p8)_3xA | tcagaggagaccacctaaatagacggcaggtcaggtcca | |
| pTS1358 | NUP43_V223V_RG- | ggtgtcgagaagaggagaacaatatgctaaatgttgttctcgtctcctcgacac | #16, |
| | V21_3xRS(20-15-15- | ccagtggccacaacatgctgtaaacggtcaccagtcagtaaaaaccctccat | #17 |
| | 20p8)_3xA | cagagaaaaggcagcggtcgccagcggg | |
| pTS1357 | NUP43_V223V_RG- | ggtgtcgagaagaggagaacaatatgctaaatgttgttctcgtctcctcgacac | #16, |
| | V21_3xRS(20-20-20- | ccagtggccacaacatgctgtaaaggcactcggtcaccagtcagaaaaaac | #17 |
| | 20p8)_3xA | cctccatcagagtccaaaaaggcagcggtcgccagcggg | |
| pTS1361 | GusB_L456L RG- | ggtgtcgagaagaggagaacaatatgctaaatgttgttctcgtctcctcgacac | #16 |
| | V21_3xRS(20-15-15- | ccagattccaggtgggacgcaaaaagaccacatcacgacaaacacgccgg | |
| | 20p8)_3xA | gacactcaaagcaccaagccagcgaagcag | |
| pTS1360 | GusB_L456L RG- | ggtgtcgagaagaggagaacaatatgctaaatgttgttctcgtctcctcgacac | #16 |
| | V21_3xRS(20-20-20- | ccagattccaggtgggacgcaaaagccacagaccacatcacgacaaacac | |
| | 20p8)_3xA | gccgggacactcatcgaaaagcaccaagccagcgaagcag | |
| pTS1343 | GAPDH_L157L RG- | ggtgtcgagaagaggagaacaatatgctaaatgttgttctcgtctcctcgacac | #16 |
| | V21_3xRS(18-15-15- | cggggtgccaagcagttggtgaaaggggcatcagcagagaaagcgccagc | |
| | 20p8)_3xA | atcgcccaaatggagggatctcgctcct | |
| pTS1345 | GAPDH_L157L_RG- | ggtgtcgagaagaggagaacaatatgctaaatgttgttctcgtctcctcgacac | #16 |
| | V21_3xRS(20-20-20- | cggggtgccaagcagttggtgaaagggggcatcagcagagggggaaaactc | |
| | 20p8)_3xA | agcgccagcatcgcccaaacgaacaggaggagcagagag | |
| pTS1351 | PAICS_V269_RG- | Ggtgtcgagaagaggagaacaatatgctaaatgttgttctcgtctcctcgacac | #16 |
| | V21_3xRS(19-15-15- | ctcaacaccacaaccctgcacaaagcaacccactcaaagaaaagtctcca | |
| | 20p8)_3xA | ggaatcaaaataacatcagcaagaacaat | |
| pTS1352 | PAICS_V269V_RG- | ggtgtcgagaagaggagaacaatatgctaaatgttgttctcgtctcctcgacac | #16 |
| | V21_3xRS(20-20-20- | ctcaacaccacaaccctgcacaaaggccagagtctccaggaatcaaaaga | |
| | 20p8)_3xA | agtccagcaaagcaaaaaaaccgcgccgggaagcgaggca | |
| pTS1347 | GPI_V627V_RG- | ggtgtcgagaagaggagaacaatatgctaaatgttgttctcgtctcctcgacac | #16 |
| | V21_3xRS(20-15-15- | ctgccgtccaccaggatgggtaaacacggctcgaccctcaaagaacatccg | |
| | 20p8)_3xA | ctcccgaaacgcatcacgtcctccgtcac | |
| pTS1348 | GPI_V627V_RG- | ggtgtcgagaagaggagaacaatatgctaaatgttgttctcgtctcctcgacac | #16 |
| | V21_3xRS(20-20-20- | ctgccgtccaccaggatgggtaaagcagcacggctcgaccctcgaaaccgg | |
| | 20p8)_3xA | gcggcctccacgccccaaaatcacgtcctccgtcaccag | |
| pTS1353 | PDE4D_L291L_RG- | Ggtgtcgagaagaggagaacaatatgctaaatgttgttctcgtctcctcgacac | #16 |
| | V21_3xRS(19-15-15- | cgggtctccagctggtccagaaaagctcctccagggtctaaaaatcaagtcat | |
| | 20p8)_3xA | ctccaaatgtccacatcaaaacgcct | |
| pTS1354 | PDE4D_L291L_RG- | ggtgtcgagaagaggagaacaatatgctaaatgttgttctcgtctcctcgacac | #16 |
| | V21_3xRS(20-20-20- | cgggtctccagctggtccagaaaacagctcctccagggtctcgcaaatcaca | |
| | 20p8)_3xA | atcaagtcatctccgaaaaagggactccgtcccgcaga | |
| pTS1193 | RAB7A_3′UTR_TAG#1_ | gtctttgataaaaggcgtacataattcttgtgtctactgtacagaatactgccgc | #17, |
| | 111p56_LEAPER | cagctggatttcccaattctgagtaacactctgcaatccaaacagggttcaaccc | #20, |
| | | t | #21 |
| pTS1216 | RAB7A_3′UTR_TAG#1_ | Gtctttgagaaaaggcggacagaagtctggtgtcgacgggacagaagacgg | #17 |
| | 111p56_LEAPER_AG20 | ccgccagctggagggcccaaggcggaggaacactcggcaatccaaacagg | |
| | | gggcaaccct | |
| pTS1194 | NUP43_V233V_111p56_ | tagtaccttgtctaacatcccaaatactcaacattccatcttggccaccagtagc | #17 |
| | LEAPER | cacaacatgctgttggttgggatgtctatcaacacagtggagtggcactcggtc | |
| | | ac | |
| pTS1217 | NUP43_V233V_111p56_ | Tagtaccttgtctaacatcccaaagactcaacattccatcgtggccaccagga | #17 |
| | LEAPER_AG6 | gccacaacatgctggggggggatgtcgatcaacacagtggagtggcactcg | |
| | | gtcac | |
| pTS1363 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaaaccaaccccgtcaagaaaaatcaggga | |
| | 20p8)_3xA_Placement_ | tctcccaaattacaaatcaacact | |
| | C | | |
| pTS1364 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaagggctcaaaagagccaaattctcaccgc | |
| | 20p8)_3xA_Placement_ | agataaaactcagccaagaaaca | |
| | D | | |
| pTS1365 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaagacgtcaaaatcaaaaaattcccaagaa | |
| | 20p8)_3xA_Placement_ | aatctaaattcaaaggcaactct | |
| | E | | |
| pTS1366 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaaaccaaccccgtcaagaaaccaccaca | |
| | 20p8)_3xA_Placement_ | cccagccaaaaggcaggagaatcac | |
| | F | | |
| pTS1367 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaactcagccaagaaacaaaaaacaaaac | |
| | 20p8)_3xA_Placement_ | acacacaaaacagagatcgcaccac | |
| | G | | |
| pTS1369 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaatcaccagtcagtgacaaattgtctatggtt | |
| | 20p8)_3xA_Placement_ | cttaaaagagtctggttattt | |
| | H | | |
| pTS1236 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaagacgtcaaaatcaaaaaaaacacagga | |
| | 20p8)_3xA_Placement_ | aacacgaaagagacaaggcctcac | |
| | B | | |
| pTS1238 | NUP43_V223V_RG- | Ggtgtcgagaagaggagaacaatatgctaaatgttgttctcgtctcctcgacac | #12 |
| | V21_3xRS(15-15-15- | ccagtagccacaacatgctgtaaaaccatcctctccaacaaaaaaccctcca | |
| | 20p8)_3xA_Placement_ | tcagaaaacgaaagcggccgcag | |
| | A | | |
| pTS1134 | hIDUA_W402X_RG- | ggtgtcgagaagaggagaacaatatgctaaatgttgttctcgtctcctcgacac | |
| | V21_9xRS_3xA | cttcggcccagagctgctcctaaactcaggaaggcaaaagtcacgtccgccc | |
| | | tccaaaaccagcccaccagcgaaacgcccgcctccccagaaatcccagcc | |
| | | gcaaaacagaccctccgagaaaggagaccaagtccaaatcctcaaagtca | |
| | | gaaaacaagctccag | |
| pTS1382 | hIDUA_W402X_RG- | Ggtgtcgagaagaggagaacaatatgctaaatgttgttctcgtctcctcgacac | |
| | V21_3xRS(15-15-15- | cttcggcccagagctgctcctaaaacgtccgccctccacaaaccagccgca | |
| | 20p8)_3xA_Version #1 | cgcccgaaagcgcgcagaccctcc | |
| pTS1383 | hIDUA_W402X_RG- | ggtgtcgagaagaggagaacaatatgctaaatgttgttctcgtctcctcgacac | |
| | V21_4xRS(15-15-15-15- | cttcggcccagagctgctcctaaaacgtccgccctccacaaaccagccgca | |
| | 20p8)_3xA_Version #2 | cgcccgaaactcccagccgcagggaaagcgcgcagaccctcc | |
| pTS1384 | hIDUA_W402X_RG- | ggtgtcgagaagaggagaacaatatgctaaatgttgttctcgtctcctcgacac | |
| | V21_3xRS(20-15-15- | cttcggcccagagctgctcctaaagcgccagcagccccaaaaagctcagga | |
| | 20p8)_3xA_Version #3 | aggcataaaaccagcgggtccgcctcgtc | |
| pTS1385 | hIDUA_W402X_RG- | ggtgtcgagaagaggagaacaatatgctaaatgttgttctcgtctcctcgacac | |
| | V21_3xRS(20-15-15- | cttcggcccagagctgctcctaaagcgccagcagccccaaaagaccagcc | |
| | 20p8)_3xA_Version #4 | caccagcaaaccgcacgcccgcctccccag | |
| pTS1386 | hIDUA_W402X_RG- | ggtgtcgagaagaggagaacaatatgctaaatgttgttctcgtctcctcgacac | |
| | V21_3xRS(20-15-15- | cttcggcccagagctgctcctaaaacgtccgccctccacaaaccagccgca | |
| | 20p8)_3xA_Version #5 | cgcccgaaagaccctccgagcaggcatcg | |
| pTS1387 | hIDUA_W402X_RG- | ggtgtcgagaagaggagaacaatatgctaaatgttgttctcgtctcctcgacac | |
| | V21_4xRS(20-15-15-15- | cttcggcccagagctgctcctaaagcgccagcagccccaaaatccgccctc | |
| | 20p8)_3xA_Version #6 | cacggcaaagaccagcccaccagcaaaccgcacgcccgcctccccag | |
| pTS1388 | hIDUA_W402X_RG- | ggtgtcgagaagaggagaacaatatgctaaatgttgttctcgtctcctcgacac | |
| | V21_3xRS(20-20-20- | cttcggcccagagctgctcctaaatcacgtccgccctccacggcaaaaccag | |
| | 20p8)_3xA_Version #7 | cccaccagcgggtccaaagccgcacgcccgcctcccca | |
| pTS1389 | hIDUA_W402X_RG- | ggtgtcgagaagaggagaacaatatgctaaatgttgttctcgtctcctcgacac | |
| | V21_3xRS(26-14-14- | cttcggcccagagctgctcctaaaagctcaggaaggcaaaagaccagccca | |
| | 20p8)_3xA_Version #8 | ccagaaagtccagccgcacgcccgcctccccag | |
| pTS1390 | hIDUA_W402X_RG- | ggtgtcgagaagaggagaacaatatgctaaatgttgttctcgtctcctcgacac | |
| | V21_3xRS(15-15-15- | cttcggcccagagctgctcctaaagcgccagcagccccaaaatgcggggg | |
| | 20p8)_3xA_Version #9 | cgggtgaaatggaagcgcgcggtg | |
| pTS1391 | hIDUA_W402X_RG- | ctggtgggctggtccctgcctttcaccgcgcgcttccatttcacccgcccgccg | |
| | V21_3xRS(20-15-15- | catttaggagcagctctgggccgaaggtgtcgaggagacgagaacaacattta | |
| | 20p8)_3xA_Version #10 | gcatattgttctcctcttctcgacacc | |
| pTS1402 | Luciferase_W417X RG- | ggtgtcgagaagaggagaacaatatgctaaatgttgttctcgtctcctcgacac | #18 |
| | V21_3xRS(15-15-20p8- | ctcgtcctcgtcccagaaagtgcagccagccgtccttgtaaagtccaagtcca | |
| | 15)_3xA | ccacaaaacgcacagctcgccg | |
| pTS1403 | Luciferase_W417X RG- | ggtgtcgagaagaggagaacaatatgctaaatgttgttctcgtctcctcgacac | #18 |
| | V21_3xRS(15-20p8-15- | ctcagccggtccacgaaaatcgtcctcgtcccagaaagtgcagccagccgtc | |
| | 15)_3xA | cttgtaaagtccaagtccaccac | |
| | hIDUA_W402X_RG- | mG*mG*mG*gucgagaagaggagaacaauaugcuaaauguuguu | #19 |
| | V21_3xRS(20-20p8-25- | cucgucuccucgacaccgccaggacgcccaccgugugaaacugucca | |
| | 20)_3xA_ASO | ggacggucccggccugcgaaauucggcccagagcugcuc*c*u*a*a* | |
| | | a*g*u*g*g*g*g*u*c*g*u*u*g*c*c*c*a*g*mC*mC*mG | |

Legend for ASO modifications:
mN = 2′O-methyl
* = Phosphorothioate linkage

### Primer List:

| Primer # | Primer Name | Sequence 5′→3′ |
|---|---|---|
| Primer | Fluc_W417X_Universal_f | acttggacaccggtaagacac |
| 2898 | W | |
| Primer | Fluc_W417X_Universal | acgatgaagaagtgctcgtcc |
| 2899 | bw | |
| 3850 | Fluc_W417X_Nested_fw | tgggtgtgaaccagcgcg |
| 3851 | Fluc_W417X_Nested_bw | gtcccagtaggcgatgtcg |

1. Artificial nucleic acid for site-directed editing of a target RNA, the artificial nucleic acid comprising in 5′ to 3′ direction or 3′ to 5′ direction:
a) a first recruiting moiety capable of recruiting a deaminase, wherein the first recruiting moiety comprises at least one recruitment sequence, which binds to a first region in the target RNA;
b) a targeting sequence which comprises a nucleic acid sequence complementary to or at least partially complementary to a target sequence in the target RNA comprising one or more nucleotides to be edited, and
c) a second recruiting moiety capable of recruiting a deaminase,
wherein the first region in the target RNA and the target sequence in the target RNA are separated by at least one nucleotide, which is not bound by the at least one recruitment sequence and which is not complementary to the targeting sequence of the artificial nucleic acid.

2. The artificial nucleic acid according to claim 1, wherein the at least one recruitment sequence comprises:
(i) a nucleic acid sequence complementary to or at least partially complementary to the first region in the target RNA; or
(ii) at least 10, preferably at least 15, more preferably at least 20, nucleotides, optionally wherein the at least one recruitment sequence comprises 10 to 200, preferably 20 to 100, nucleotides.

3. The artificial nucleic acid according to claim 1, wherein the first recruiting moiety comprises a cluster of recruitment sequences comprising at least two recruitment sequences which are linked via a nucleotide linker,
optionally wherein the cluster of recruitment sequences comprises at least 3, preferably 3 to 10, recruitment sequences, and
optionally wherein the nucleotide linker linking the recruitment sequences comprises at least 1 nucleotide, preferably 2 to 6 nucleotides, optionally wherein the nucleotides are adenosine nucleotides.

4.-8. (canceled)

9. The artificial nucleic acid according to claim 3, wherein a first recruitment sequence binds to a first region in the target RNA and preferably comprises a nucleic acid sequence complementary to or at least partially complementary to the first region in the target RNA, and a second or further recruitment sequence binds to a second or further region in the target RNA and preferably comprises a nucleic acid sequence complementary to or at least partially complementary to the second or further region in the target RNA.

10. The artificial nucleic acid according to claim 1, wherein the first region in the target RNA and/or the second and/or further region in the target RNA do not comprise any editable adenosine nucleotide(s).

11. The artificial nucleic acid according to claim 1, wherein the artificial nucleic acid comprises:
(i) a nucleotide spacer between the first recruiting moiety and the targeting sequence, optionally wherein the nucleotide spacer comprises at least 1 nucleotide, preferably 2 to 6 nucleotides, optionally wherein the nucleotides are adenosine nucleotides; or
(ii) is an RNA or RNA analog, or an endogenously expressible RNA.

12.-13. (canceled)

14. The artificial nucleic acid according to claim 1, wherein the targeting sequence comprises:
(i) at least 10 nucleotides, preferably 10 to 50, more preferably 16 to 40 nucleotides; or
(ii) at the position corresponding to a nucleotide to be edited, preferably an adenosine to be edited, a cytidine nucleotide mismatching the adenosine to be edited.

15. (canceled)

16. The artificial nucleic acid according to claim 14, wherein the cytidine nucleotide mismatching the adenosine to be edited is positioned at least 6 nucleotides distant from either the 5′ or 3′ terminus of the targeting sequence.

17. The artificial nucleic acid according to claim 1, wherein at least one of the first recruiting moiety and the second recruiting moiety comprises a nucleic acid sequence capable of binding to a deaminase, preferably an adenosine deaminase, without binding to the target RNA.

18. The artificial nucleic acid according to claim 17, wherein the nucleic acid sequence is capable of binding to the dsRNA binding domain of a deaminase, preferably an adenosine deaminase, optionally wherein the nucleic acid sequence is capable of:
i) binding to ADAR1 or ADAR2, preferably ADAR1, preferably human ADAR1, in particular ADAR1p110, or
ii) intramolecular base pairing, preferably capable of forming a stem-loop structure.

19.-20. (canceled)

21. The artificial nucleic acid according to claim 19, wherein the stem-loop structure comprises a double-helical stem comprising at least two mismatches, and a loop consisting of from 3 to 8, preferably from 4 to 6, more preferably 5, nucleotides, wherein the loop preferably comprises the nucleic acid sequence GCUAA or GCUCA.

22. The artificial nucleic acid according to claim 21, wherein the nucleic acid sequence comprises the nucleotide sequence
(i)
5′-GGUGU CGAGA AGAGG AGAAC AAUAU GCUAA AUGUU
GUUCU CGUCU CCUCG ACACC-3′,
or
(ii)
5′-GUG GAA UAG UAU AAC AAU AUG CUA AAU GUU GUU
AUA GUA UCC CAC-3′.

23. (canceled)

24. The artificial nucleic acid according to claim 1, wherein the second recruiting moiety
(i is a recruiting moiety as defined with respect to the first recruiting moiety in claim 1, optionally wherein the artificial nucleic acid comprises a nucleotide spacer between the targeting sequence and the second recruiting moiety; or
(ii) comprises a nucleic acid sequence capable of binding to a deaminase.

25.-27. (canceled)

28. The artificial nucleic acid according to claim 24, which comprises in 5′ to 3′ direction or 3′ to 5′ direction:
a) a first recruiting moiety comprising at least one recruitment sequence which binds and preferably is complementary or at least partially complementary to a first region in the target RNA, and preferably comprising a cluster of recruitment sequences which bind and preferably are complementary or at least partially complementary to a first and further regions of the target RNA;
b) a targeting sequence which comprises a nucleic acid sequence complementary or at least partially complementary to a target sequence in the target RNA comprising one or more nucleotides to be edited, and
c) a second recruiting moiety comprising a nucleic acid sequence capable of binding a deaminase; optionally wherein the second recruiting moiety comprises at least one further recruitment sequence which binds and preferably is complementary or at least partially complementary to a further region in the target RNA, wherein the further recruitment sequence(s) preferably is/are depleted from uridine bases, unless they are either within 5 nt from either 5′ or 3′end of a recruitment sequence or in a 5′-NUS context, wherein S═C or G.

29. The artificial nucleic acid according to claim 1, wherein the first recruitment sequence and/or further recruitment sequence(s) is/are depleted from uridine bases unless they are either within 5 nt from either 5′ or 3′end of a recruitment sequence or in a 5′-NUS context, wherein S═C or G.

30. (canceled)

31. An artificial nucleic acid for site-directed editing of a target RNA, the artificial nucleic acid comprising in 5′ to 3′ direction or 3′ to 5′ direction:
a) a first recruiting moiety capable of recruiting a deaminase, wherein the first recruiting moiety comprises at least one recruitment sequence, which binds to a first region in the target RNA;
b) a targeting sequence which comprises a nucleic acid sequence complementary to or at least partially complementary to a target sequence in the target RNA comprising one or more nucleotides to be edited, and
c) a second recruiting moiety capable of recruiting a deaminase, wherein the second recruiting moiety preferably comprises a nucleic acid sequence capable of binding a deaminase.

32.-51. (canceled)

52. Cell comprising the artificial nucleic acid according to claim 1.

53. Composition comprising the artificial nucleic acid according to claim 1, and an additional excipient.

54.-61. (canceled)

62. A method of treating of preventing a disease or a disorder in a subject, the method comprising administering an effective amount of the artificial nucleic acid according to claim 1.

63. The method according to claim 62, wherein the disease or the disorder is
(i) a genetic disease, or
(ii) selected from the group consisting of infectious diseases, tumour diseases, cardiovascular diseases, autoimmune diseases, allergies and neurological diseases or disorders.

64. (canceled)


SEQUENCE LISTING (92 sequences)

SEQ ID NO: 1 - DNA, 75 bp, Artificial Sequence
  R/G-V21 motif, targeting sequence.
  Luciferase_W417X_RG-V21_0xRS_20p8 (1)..(75): Used in figure #1 & #7
ggtgtcgagaagaggagaacaatatgctaaatgttgttctcgtctcctcgacaccgtgcagccagccgtccttgt

SEQ ID NO: 2 - DNA, 123 bp, Artificial Sequence
  R/G-V21 motif, targeting sequence, recruitment sequences & linker.
  Luciferase_W417X_RG-V21_3xRS(15-13-11-20p8)_3xA (1)..(123): Used in figure #1, #2, #3, #4, #6, #7, #8, #9, #13
ggtgtcgagaagaggagaacaatatgctaaatgttgttctcgtctcctcgacaccgtgcagccagccgtccttgtaaacgcacagctcgaaagtccaagtccaccaaaaagaagggcaccacc

SEQ ID NO: 3 - DNA, 206 bp, Artificial Sequence
  R/G-V21 motif, targeting sequence, recruitment sequences & linker.
  Luciferase_W417X_RG-V21_8xRS(11-15-15-15-12-15-13-11-20p8)_3xA (1)..(206): Used in figure #1, #6, #7
ggtgtcgagaagaggagaacaatatgctaaatgttgttctcgtctcctcgacaccgtgcagccagccgtccttgtaaacgcacagctcgaaagtccaagtccaccaaaaagaagggcaccaccaaactcctcctcgaaaaaaaagccgcagatcaaaaacgaagctctcgaaaccacagccacaccgaaaacacaccacgatccga

SEQ ID NO: 4 - DNA, 95 bp, Artificial Sequence
  R/G-V20 motif, targeting sequence.
  Luciferase_W417X_RG-V20_0xRS_16p8_BoxB (1)..(95): Used in figure #2
gtggtcgagaagaggagaacaatatgctaaatgttgttctcgtctcctcgaccacgtgcagccagccgtcctctagagggccctgaagagggccc

SEQ ID NO: 5 - DNA, 223 bp, Artificial Sequence
  R/G-V21 motif, targeting sequence, recruitment sequences & linker.
  AHI_W725X_RG-V21_9xRS_20p8 (1)..(223)
  AHI_W725X_RG-V21_9xRS_20p8 (1)..(223): Used in figure #5
ggtgtcgagaagaggagaacaatatgctaaatgttgttctcgtctcctcgacaccaactttccatatccgtatcaaaaaaacaaaagaaggaaaaaaacgtccagaagaaagagaaatcaagaaaagaagagccatcaaaacctcaacaacacaaaaagaaatcaagaataaacctcgaagcaaaaaagaaacaggccgtccacaaaggtcatcatcaagcaa

SEQ ID NO: 6 - DNA, 225 bp, Artificial Sequence
  R/G-V21 motif, targeting sequence, recruitment sequences & linker.
  BMPR2_W298X_RG-V21_9xRS_20p8 (1)..(225): Used in figure #5
ggtgtcgagaagaggagaacaatatgctaaatgttgttctcgtctcctcgacaccgcttacccagtcacttgtgtaaaactccatcacaaaaactctctcatctccaacaaatccatcaaaggcactcaaagcaaaggaaaacacaaagacgctcatccaagaaaacctcggccaatcagaaaatcaaaacagcaaaaacagcagaaacgaaaagaccaacatcc

SEQ ID NO: 7 - DNA, 225 bp, Artificial Sequence
  R/G-V21 motif, targeting sequence, recruitment sequences & linker.
  COL3A1_W1278X_RG-V21_9xRS_20p8 (1)..(225): Used in figure #5
ggtgtcgagaagaggagaacaatatgctaaatgttgttctcgtctcctcgacaccgtcaacccagtattctccacaaaagaaccatcagaaagggcaaaaccgcaaaacctccaatcccagcaaaacaacaccaccacagcaaaaccctcagatcctcaaaggtccaacaggtcctcaaacagcagctccacgaaaggccagcagggccacaaacaccacgatca

SEQ ID NO: 8 - DNA, 225 bp, Artificial Sequence
  R/G-V21 motif, targeting sequence, recruitment sequences & linker.
  FANCC_W506X_RG-V21_9xRS_20p8 (1)..(225): Used in figure #5
ggtgtcgagaagaggagaacaatatgctaaatgttgttctcgtctcctcgacaccgacatcccaggcgatcgtgtaaagcctcccatcacaaatctcagcccatcctccaaagaaccagctctcaaagaaacaaaccgcagcaaatccagggccccatcgaaactcatcaacaacccggaaagccgaagccagaaaacctcaaagaactcaaaggacacaaactc

SEQ ID NO: 9 - DNA, 211 bp, Artificial Sequence
  R/G-V21 motif, targeting sequence, recruitment sequences & linker.
  MYBPC3_W1098X_RG-V21_9xRS_20p8 (1)..(211): Used in figure #5
ggtgtcgagaagaggagaacaatatgctaaatgttgttctcgtctcctcgacaccgtacccccagagctccgtgtaaatccactccagaaaacgtcagtcacccgaaagcaccgtcaccaaatcacctcctcgaaatcacaggctccccgacaaagaaaagcagccgaaacagccacccactaaatcccacgcgctaaaagacgcgcatct

SEQ ID NO: 10 - DNA, 173 bp, Artificial Sequence
  R/G-V21 motif, targeting sequence, recruitment sequences & linker.
  IL2RG_W237X_RG-V21_6xRS_20p8 (1)..(173): Used in figure #5
gggagtggggctgtttgtgttttgtgttctttcttctggctgttttcggactgactgggactttgtgtggatgggctttgtttcgtgttcggagctttgtgctcagcattggagtgaaggtgtcgaggagacgagaacaacatttagcatattgttctcctcttctcgacacc

SEQ ID NO: 11 - DNA, 123 bp, Artificial Sequence
  R/G-V21 motif, targeting sequence, recruitment sequences & linker.
  PINK1_W437X_RG-V21_3xRS_20p8 (1)..(123): Used in figure #5
ggtgtcgagaagaggagaacaatatgctaaatgttgttctcgtctcctcgacacccactgcccaggcatcagcctaaaccgccccgatccacgaaatctcatcagccagaaaatcaccagcca

SEQ ID NO: 12 - DNA, 89 bp, Artificial Sequence
  R/G-V21 motif, targeting sequence, recruitment sequences & linker.
  Luciferase_W417X_RG-V21_1xRS_20p8 (1)..(89): Used in figure #7
ggtgtcgagaagaggagaacaatatgctaaatgttgttctcgtctcctcgacaccgtgcagccagccgtccttgtaaacgcacagctcg

SEQ ID NO: 13 - DNA, 105 bp, Artificial Sequence
  R/G-V21 motif, targeting sequence, recruitment sequences & linker.
  Luciferase_W417X_RG-V21_2xRS_20p8 (1)..(105): Used in figure #7
ggtgtcgagaagaggagaacaatatgctaaatgttgttctcgtctcctcgacaccgtgcagccagccgtccttgtaaacgcacagctcgaaagtccaagtccacc

SEQ ID NO: 14 - DNA, 170 bp, Artificial Sequence
  R/G-V21 motif, targeting sequence, recruitment sequences & linker.
  Luciferase_W417X_RG-V21_6xRS_20p8 (1)..(170): Used in figure #7
ggtgtcgagaagaggagaacaatatgctaaatgttgttctcgtctcctcgacaccgtgcagccagccgtccttgtaaacgcacagctcgaaagtccaagtccaccaaaaagaagggcaccaccaaactcctcctcgaaaaaaaagccgcagatcaaaaacgaagctctcg

SEQ ID NO: 15 - DNA, 384 bp, Artificial Sequence
  R/G-V21 motif, targeting sequence, recruitment sequences & linker.
  Luciferase_W417X_RG-V21_20xRS_20p8 (1)..(384): Used in figure #7
ggtgtcgagaagaggagaacaatatgctaaatgttgttctcgtctcctcgacaccgtgcagccagccgtccttgtaaacgcacagctcgaaagtccaagtccaccaaaaagaagggcaccaccaaactcctcctcgaaaaaaaagccgcagatcaaaaacgaagctctcgaaaccacagccacaccgaaaacacaccacgatccgaaaagaacgctcatctcaaactcgccggcggtcccaaactcctccacgtctccaaaatcaacgaacgaaaagaaaagaatccaaaacaacgtcaggaaatctccaaaaccaaaatcaatcacatcaaacaccccaatcaaaaacaccgcgcaaagaatcaagaacaaaagcccaccact

SEQ ID NO: 16 - DNA, 105 bp, Artificial Sequence
  R/G-V21 motif, targeting sequence, recruitment sequences & linker.
  Luciferase_W417X_RG-V21_3xRS(7-7-7-20p8) (1)..(105): Used in figure #7
  Luciferase_W417X_RG-V21_3xRS(7-7-7-20p8) (1)..(105): Used in figure #8
ggtgtcgagaagaggagaacaatatgctaaatgttgttctcgtctcctcgacaccgtgcagccagccgtccttgtaaacctcgggaaaaagtccaaaagaagaag

SEQ ID NO: 17 - DNA, 111 bp, Artificial Sequence
  R/G-V21 motif, targeting sequence, recruitment sequences & linker.
  Luciferase_W417X_RG-V21_3xRS(9-9-9-20p8) (1)..(111): Used in figure #8
ggtgtcgagaagaggagaacaatatgctaaatgttgttctcgtctcctcgacaccgtgcagccagccgtccttgtaaaacgcacagcaaacacacccagaaatccaccacc

SEQ ID NO: 18 - DNA, 117 bp, Artificial Sequence
  R/G-V21 motif, targeting sequence, recruitment sequences & linker.
  Luciferase_W417X_RG-V21_3xRS(11-11-11-20p8) (1)..(117): Used in figure #8
ggtgtcgagaagaggagaacaatatgctaaatgttgttctcgtctcctcgacaccgtgcagccagccgtccttgtaaacgcacagctcgaaacaagtccaccaaaaagggcaccacc

SEQ ID NO: 19 - DNA, 123 bp, Artificial Sequence
  R/G-V21 motif, targeting sequence, recruitment sequences & linker.
  Luciferase_W417X_RG-V21_3xRS(13-13-13-20p8) (1)..(123): Used in figure #8
ggtgtcgagaagaggagaacaatatgctaaatgttgttctcgtctcctcgacaccgtgcagccagccgtccttgtaaaacggacgcacagcaaaccaagtccaccacaaaagaagggcaccac

SEQ ID NO: 20 - DNA, 129 bp, Artificial Sequence
  R/G-V21 motif, targeting sequence, recruitment sequences & linker.
  Luciferase_W417X_RG-V21_3xRS(15-S-15-S-15-S-20p8) (1)..(129): Used in figure #8, #10, #11, #18
ggtgtcgagaagaggagaacaatatgctaaatgttgttctcgtctcctcgacaccgtgcagccagccgtccttgtaaaacgcacagctcgccgaaagtccaagtccaccacaaaaagaagggcaccacc

SEQ ID NO: 21 - DNA, 114 bp, Artificial Sequence
  R/G-V21 motif, targeting sequence, recruitment sequences & linker.
  Luciferase_W417X_RG-V21_3xRS(15-13-11-20p8)_0xA (1)..(114): Used in figure #9
ggtgtcgagaagaggagaacaatatgctaaatgttgttctcgtctcctcgacaccgtgcagccagccgtccttgtcgcacagctcggtccaagtccaccaagaagggcaccacc

SEQ ID NO: 22 - DNA, 117 bp, Artificial Sequence
  R/G-V21 motif, targeting sequence, recruitment sequences & linker.
  Luciferase_W417X_RG-V21_3xRS(15-13-11-20p8)_1xA (1)..(117): Used in figure #9
ggtgtcgagaagaggagaacaatatgctaaatgttgttctcgtctcctcgacaccgtgcagccagccgtccttgtacgcacagctcgagtccaagtccaccaaagaagggcaccacc

SEQ ID NO: 23 - DNA, 120 bp, Artificial Sequence
  R/G-V21 motif, targeting sequence, recruitment sequences & linker.
  Luciferase_W417X_RG-V21_3xRS(15-13-11-20p8)_2xA (1)..(120): Used in figure #9
ggtgtcgagaagaggagaacaatatgctaaatgttgttctcgtctcctcgacaccgtgcagccagccgtccttgtaacgcacagctcgaagtccaagtccaccaaaagaagggcaccacc

SEQ ID NO: 24 - DNA, 126 bp, Artificial Sequence
  R/G-V21 motif, targeting sequence, recruitment sequences & linker.
  Luciferase_W417X_RG-V21_3xRS(15-13-11-20p8)_4xA (1)..(126): Used in figure #9
ggtgtcgagaagaggagaacaatatgctaaatgttgttctcgtctcctcgacaccgtgcagccagccgtccttgtaaaacgcacagctcgaaaagtccaagtccaccaaaaaagaagggcaccacc

SEQ ID NO: 25 - DNA, 129 bp, Artificial Sequence
  R/G-V21 motif, targeting sequence, recruitment sequences & linker.
  Luciferase_W417X_RG-V21_3xRS(15-13-11-20p8)_5xA (1)..(129): Used in figure #9
ggtgtcgagaagaggagaacaatatgctaaatgttgttctcgtctcctcgacaccgtgcagccagccgtccttgtaaaaacgcacagctcgaaaaagtccaagtccaccaaaaaaagaagggcaccacc

SEQ ID NO: 26 - DNA, 143 bp, Artificial Sequence
  R/G-V21 motif, targeting sequence, recruitment sequences & linker.
  Luciferase_W417X_RG-V21_3xRS(15-13-11-20p8)_10xA (1)..(143): Used in figure #9
ggtgtcgagaagaggagaacaatatgctaaatgttgttctcgtctcctcgacaccgtgcagccagccgtccttgtaaaaaaaaaacgcacagctcgaaaaaaaaaagtccaagtccaccaaaaaaaaaaaagaagggcaccac

SEQ ID NO: 27 - DNA, 129 bp, Artificial Sequence
  R/G-V21 motif, targeting sequence, recruitment sequences & linker.
  Luciferase_W417X_RG-V21_3xRS(15-L-15-S-15-S-20p8) (1)..(129): Used in figure #10
ggtgtcgagaagaggagaacaatatgctaaatgttgttctcgtctcctcgacaccgtgcagccagccgtccttgtaaaacgcacagctcgccgaaagtccaagtccaccacaaacgaaagccgcagatc

SEQ ID NO: 28 - DNA, 129 bp, Artificial Sequence
  R/G-V21 motif, targeting sequence, recruitment sequences & linker.
  Luciferase_W417X_RG-V21_3xRS(15-L-15-L-15-S-20p8) (1)..(129): Used in figure #10
ggtgtcgagaagaggagaacaatatgctaaatgttgttctcgtctcctcgacaccgtgcagccagccgtccttgtaaaacgcacagctcgccgaaacgaaagccgcagatcaaaccacagccacaccga

SEQ ID NO: 29 - DNA, 129 bp, Artificial Sequence
  R/G-V21 motif, targeting sequence, recruitment sequences & linker.
  Luciferase_W417X_RG-V21_3xRS(15-S-15-L-15-S-20p8) (1)..(129): Used in figure #10
ggtgtcgagaagaggagaacaatatgctaaatgttgttctcgtctcctcgacaccgtgcagccagccgtccttgtaaaacgcacagctcgccgaaacgaaagccgcagatcaaagaacatgccgaagcc

SEQ ID NO: 30 - DNA, 129 bp, Artificial Sequence
  R/G-V21 motif, targeting sequence, recruitment sequences & linker.
  Luciferase_W417X_RG-V21_3xRS(15-S-15-S-15-L-20p8) (1)..(129): Used in figure #10
ggtgtcgagaagaggagaacaatatgctaaatgttgttctcgtctcctcgacaccgtgcagccagccgtccttgtaaacgaaagccgcagatcaaagaacatgccgaagccaaaatggggtcgcgggca

SEQ ID NO: 31 - DNA, 129 bp, Artificial Sequence
  R/G-V21 motif, targeting sequence, recruitment sequences & linker.
  Luciferase_W417X_RG-V21_3xRS(15-S-15-L-15-L-20p8) (1)..(129): Used in figure #10
ggtgtcgagaagaggagaacaatatgctaaatgttgttctcgtctcctcgacaccgtgcagccagccgtccttgtaaacgaaagccgcagatcaaaccacagccacaccgaaaacacccaacacgggca

SEQ ID NO: 32 - DNA, 129 bp, Artificial Sequence
  R/G-V21 motif, targeting sequence, recruitment sequences & linker.
  Luciferase_W417X_RG-V21_3xRS(15-L-15-S-15-L-20p8) (1)..(129): Used in figure #10
ggtgtcgagaagaggagaacaatatgctaaatgttgttctcgtctcctcgacaccgtgcagccagccgtccttgtaaacgaaagccgcagatcaaagaacatgccgaagccaaacacccaacacgggca

SEQ ID NO: 33 - DNA, 129 bp, Artificial Sequence
  R/G-V21 motif, targeting sequence, recruitment sequences & linker.
  Luciferase_W417X_RG-V21_3xRS(15-L-15-L-15-L-20p8) (1)..(129): Used in figure #10, #11
ggtgtcgagaagaggagaacaatatgctaaatgttgttctcgtctcctcgacaccgtgcagccagccgtccttgtaaacgaaagccgcagatcaaaccacagccacaccgaaaactcgctcaacgaacg

SEQ ID NO: 34 - DNA, 134 bp, Artificial Sequence
  R/G-V21 motif, targeting sequence, recruitment sequences & linker.
  Luciferase_W417X_RG-V21_3xRS(20-S-15-S-15-S-20p8) (1)..(134): Used in figure #11, #14
ggtgtcgagaagaggagaacaatatgctaaatgttgttctcgtctcctcgacaccgtgcagccagccgtccttgtaaaacgcacagctcgccgaaagtccaagtccaccacaaacctcgaagaagggcaccacc

SEQ ID NO: 35 - DNA, 134 bp, Artificial Sequence
  R/G-V21 motif, targeting sequence, recruitment sequences & linker.
  Luciferase_W417X_RG-V21_3xRS(15-S-20-S-15-S-20p8) (1)..(134): Used in figure #11
ggtgtcgagaagaggagaacaatatgctaaatgttgttctcgtctcctcgacaccgtgcagccagccgtccttgtaaaacgcacagctcgccgaaaccggtgtccaagtccaccacaaaaagaagggcaccacc

SEQ ID NO: 36 - DNA, 134 bp, Artificial Sequence
  R/G-V21 motif, targeting sequence, recruitment sequences & linker.
  Luciferase_W417X_RG-V21_3xRS(15-S-15-S-20-S-20p8) (1)..(134): Used in figure #11
ggtgtcgagaagaggagaacaatatgctaaatgttgttctcgtctcctcgacaccgtgcagccagccgtccttgtaaacacggacgcacagctcgccgaaagtccaagtccaccacaaaaagaagggcaccacc

SEQ ID NO: 37 - DNA, 134 bp, Artificial Sequence
  R/G-V21 motif, targeting sequence, recruitment sequences & linker.
  Luciferase_W417X_RG-V21_3xRS(20-L-15-L-15-L-20p8) (1)..(134): Used in figure #11
ggtgtcgagaagaggagaacaatatgctaaatgttgttctcgtctcctcgacaccgtgcagccagccgtccttgtaaacgaaagccgcagatcaaaccacagccacaccgaaaagagaactcgctcaacgaacg

SEQ ID NO: 38 - DNA, 134 bp, Artificial Sequence
  R/G-V21 motif, targeting sequence, recruitment sequences & linker.
  Luciferase_W417X_RG-V21_3xRS(15-L-20-L-15-L-20p8) (1)..(134): Used in figure #11
ggtgtcgagaagaggagaacaatatgctaaatgttgttctcgtctcctcgacaccgtgcagccagccgtccttgtaaacgaaagccgcagatcaaaggggccacagccacaccgataaactcgctcaacgaacg

SEQ ID NO: 39 - DNA, 134 bp, Artificial Sequence
  R/G-V21 motif, targeting sequence, recruitment sequences & linker.
  Luciferase_W417X_RG-V21_3xRS(15-L-15-L-20-L-20p8) (1)..(134): Used in figure #11
ggtgtcgagaagaggagaacaatatgctaaatgttgttctcgtctcctcgacaccgtgcagccagccgtccttgtaaacgacccgaaagccgcagatcaaaccacagccacaccgaaaactcgctcaacgaacg

SEQ ID NO: 40 - DNA, 171 bp, Artificial Sequence
  R/G-V21 motif, targeting sequence, recruitment sequences & linker.
  Luciferase_W417X_RG-V21_3xRS(RS#1-3mask_15-13-11-20p8) (1)..(171): Used in figure #13
ggtgtcgagaagaggagaacaatatgctaaatgttgttctcgtctcctcgacaccgtgcagccagccgtccttgtaaacgcacagctcgaaagtccaagtccaccaaaaagaagggcaccaccaaaggtggtgcccttcttaaaggtggacttggacaaacgagctgtgcg

SEQ ID NO: 41 - DNA, 141 bp, Artificial Sequence
  R/G-V21 motif, targeting sequence, recruitment sequences & linker.
  Luciferase_W417X_RG-V21_3xRS(RS#3mask_15-13-11-20p8) (1)..(141): Used in figure #13
ggtgtcgagaagaggagaacaatatgctaaatgttgttctcgtctcctcgacaccgtgcagccagccgtccttgtaaacgcacagctcgaaagtccaagtccaccaaaaagaagggcaccaccaaaggtggtgcccttctt

SEQ ID NO: 42 - DNA, 157 bp, Artificial Sequence
  R/G-V21 motif, targeting sequence, recruitment sequences & linker.
  Luciferase_W417X_RG-V21_3xRS(RS#2+3mask_15-13-11-20p8) (1)..(157): Used in figure #13
ggtgtcgagaagaggagaacaatatgctaaatgttgttctcgtctcctcgacaccgtgcagccagccgtccttgtaaacgcacagctcgaaagtccaagtccaccaaaaagaagggcaccaccaaaggtggtgcccttcttaaaggtggacttggac

SEQ ID NO: 43 - DNA, 134 bp, Artificial Sequence
  R/G-V21 motif, targeting sequence, recruitment sequences & linker.
  RAB7A_3′UTR_TAG#1_RG-V21_3xRS(20-15-15-20p8)_3xA (1)..(134): Used in figure #15, #17
ggtgtcgagaagaggagaacaatatgctaaatgttgttctcgtctcctcgacaccctgccgccagctggatttccaaattcaaccctccacctaaatacaaaactcagcctaaataaaaagaccacaagaccaa

SEQ ID NO: 44 - DNA, 127 bp, Artificial Sequence
  R/G-V21 motif, targeting sequence, recruitment sequences & linker.
  RAB7A_3′UTR_TAG#1_RG-V21_3xRS(19-13-11-20p8)_3xA (1)..(127): Used in figure #15, #17, #20, #21
ggtgtcgagaagaggagaacaatatgctaaatgttgttctcgtctcctcgacaccctgccgccagctggatttccaaatcaaccctccaccaaaacaaaactcagaaaaaaaagaccacaagaccaa

SEQ ID NO: 45 - DNA, 134 bp, Artificial Sequence
  R/G-V21 motif, targeting sequence, recruitment sequences & linker.
  Beta-Actin_3′UTR_TAG#1_RG-V21_3xRS(20-15-15-20p8)_3xA (1)..(134): Used in figure #15
ggtgtcgagaagaggagaacaatatgctaaatgttgttctcgtctcctcgacaccacgcaaccaagtcatagtccaaaggaggggccggactcaaaccgccgatccacacgaaatgcgctcaggaggagcaatg

SEQ ID NO: 46 - DNA, 144 bp, Artificial Sequence
  R/G-V21 motif, targeting sequence, recruitment sequences & linker.
  Beta-Actin_3′UTR_TAG#1_RG-V21_3xRS(20-20-20-20p8)_3xA (1)..(144): Used in figure #15
ggtgtcgagaagaggagaacaatatgctaaatgttgttctcgtctcctcgacaccacgcaaccaagtcatagtccaaaagccgccgatccacacggagaaacctcagggcagcggaaccgcaaagctcgaagtccagggcgacg

SEQ ID NO: 47 - DNA, 134 bp, Artificial Sequence
  R/G-V21 motif, targeting sequence, recruitment sequences & linker.
  GAPDH_3′UTR_TAG#1_RG-V21_3xRS(20-15-15-20p8)_3xA (1)..(134): Used in figure #15
ggtgtcgagaagaggagaacaatatgctaaatgttgttctcgtctcctcgacaccaggggtccacatggcaactgaaacagggactccccagcaaagaagtcagaggagacaaatagacggcaggtcaggtcca

SEQ ID NO: 48 - DNA, 144 bp, Artificial Sequence
  R/G-V21 motif, targeting sequence, recruitment sequences & linker.
  GAPDH_3′UTR_TAG#1_RG-V21_3xRS(20-20-20-20p8)_3xA (1)..(144): Used in figure #15
ggtgtcgagaagaggagaacaatatgctaaatgttgttctcgtctcctcgacaccaggggtccacatggcaactgaaatgtggcagggactccccagcaaagaagtcagaggagaccacctaaatagacggcaggtcaggtcca

SEQ ID NO: 49 - DNA, 134 bp, Artificial Sequence
  R/G-V21 motif, targeting sequence, recruitment sequences & linker.
  NUP43_V223V_RG-V21_3xRS(20-15-15-20p8)_3xA (1)..(134): Used in figure #16, #17
ggtgtcgagaagaggagaacaatatgctaaatgttgttctcgtctcctcgacacccagtggccacaacatgctgtaaacggtcaccagtcagtaaaaaccctccatcagagaaaaggcagcggtcgccagcggg

SEQ ID NO: 50 - DNA, 144 bp, Artificial Sequence
  R/G-V21 motif, targeting sequence, recruitment sequences & linker.
  NUP43_V223V_RG-V21_3xRS(20-20-20-20p8)_3xA (1)..(144): Used in figure #16, #17
ggtgtcgagaagaggagaacaatatgctaaatgttgttctcgtctcctcgacacccagtggccacaacatgctgtaaaggcactcggtcaccagtcagaaaaaaccctccatcagagtccaaaaaggcagcggtcgccagcggg

SEQ ID NO: 51 - DNA, 134 bp, Artificial Sequence
  R/G-V21 motif, targeting sequence, recruitment sequences & linker.
  GusB_L456L_RG-V21_3xRS(20-15-15-20p8)_3xA (1)..(134): Used in figure #16
ggtgtcgagaagaggagaacaatatgctaaatgttgttctcgtctcctcgacacccagattccaggtgggacgcaaaaagaccacatcacgacaaacacgccgggacactcaaagcaccaagccagcgaagcag

SEQ ID NO: 52 - DNA, 144 bp, Artificial Sequence
  R/G-V21 motif, targeting sequence, recruitment sequences & linker.
  GusB_L456L_RG-V21_3xRS(20-20-20-20p8)_3xA (1)..(144): Used in figure #16
ggtgtcgagaagaggagaacaatatgctaaatgttgttctcgtctcctcgacacccagattccaggtgggacgcaaaagccacagaccacatcacgacaaacacgccgggacactcatcgaaaagcaccaagccagcgaagcag

SEQ ID NO: 53 - DNA, 132 bp, Artificial Sequence
  R/G-V21 motif, targeting sequence, recruitment sequences & linker.
  GAPDH_L157L_RG-V21_3xRS(18-15-15-20p8)_3xA (1)..(132): Used in figure #16
ggtgtcgagaagaggagaacaatatgctaaatgttgttctcgtctcctcgacaccggggtgccaagcagttggtgaaaggggcatcagcagagaaagcgccagcatcgcccaaatggagggatctcgctcct

SEQ ID NO: 54 - DNA, 144 bp, Artificial Sequence
  R/G-V21 motif, targeting sequence, recruitment sequences & linker.
  GAPDH_L157L_RG-V21_3xRS(20-20-20-20p8)_3xA (1)..(144): Used in figure #16
ggtgtcgagaagaggagaacaatatgctaaatgttgttctcgtctcctcgacaccggggtgccaagcagttggtgaaagggggcatcagcagagggggaaaactcagcgccagcatcgcccaaacgaacaggaggagcagagag

SEQ ID NO: 55 - DNA, 133 bp, Artificial Sequence
  R/G-V21 motif, targeting sequence, recruitment sequences & linker.
  PAICS_V269_RG-V21_3xRS(19-15-15-20p8)_3xA (1)..(133): Used in figure #16
ggtgtcgagaagaggagaacaatatgctaaatgttgttctcgtctcctcgacacctcaacaccacaaccctgcacaaagcaacccactcaaagaaaagtctccaggaatcaaaataacatcagcaagaacaat

SEQ ID NO: 56 - DNA, 144 bp, Artificial Sequence
  R/G-V21 motif, targeting sequence, recruitment sequences & linker.
  PAICS_V269V_RG-V21_3xRS(20-20-20-20p8)_3xA (1)..(144): Used in figure #16
ggtgtcgagaagaggagaacaatatgctaaatgttgttctcgtctcctcgacacctcaacaccacaaccctgcacaaaggccagagtctccaggaatcaaaagaagtccagcaaagcaaaaaaaccgcgccgggaagcgaggca

SEQ ID NO: 57 - DNA, 134 bp, Artificial Sequence
  R/G-V21 motif, targeting sequence, recruitment sequences & linker.
  GPI_V627V_RG-V21_3xRS(20-15-15-20p8)_3xA (1)..(134): Used in figure #16
ggtgtcgagaagaggagaacaatatgctaaatgttgttctcgtctcctcgacacctgccgtccaccaggatgggtaaacacggctcgaccctcaaagaacatccgctcccgaaacgcatcacgtcctccgtcac

SEQ ID NO: 58 - DNA, 144 bp, Artificial Sequence
  R/G-V21 motif, targeting sequence, recruitment sequences & linker.
  GPI_V627V_RG-V21_3xRS(20-20-20-20p8)_3xA (1)..(144): Used in figure #16
ggtgtcgagaagaggagaacaatatgctaaatgttgttctcgtctcctcgacacctgccgtccaccaggatgggtaaagcagcacggctcgaccctcgaaaccgggcggcctccacgccccaaaatcacgtcctccgtcaccag

SEQ ID NO: 59 - DNA, 133 bp, Artificial Sequence
  R/G-V21 motif, targeting sequence, recruitment sequences & linker.
  PDE4D_L291L_RG-V21_3xRS(19-15-15-20p8)_3xA (1)..(133): Used in figure #16
ggtgtcgagaagaggagaacaatatgctaaatgttgttctcgtctcctcgacaccgggtctccagctggtccagaaaagctcctccagggtctaaaaatcaagtcatctccaaatgtccacatcaaaacgcct

SEQ ID NO: 60 - DNA, 144 bp, Artificial Sequence
  R/G-V21 motif, targeting sequence, recruitment sequences & linker.
  PDE4D_L291L_RG-V21_3xRS(20-20-20-20p8)_3xA (1)..(144): Used in figure #16
ggtgtcgagaagaggagaacaatatgctaaatgttgttctcgtctcctcgacaccgggtctccagctggtccagaaaacagctcctccagggtctcgcaaatcacaatcaagtcatctccgaaaaagggactccgtcccgcaga

SEQ ID NO: 61 - DNA, 111 bp, Artificial Sequence
  Simple and unstructured LEAPER gRNA that is reverse complementary to the target mRNA, but contains a C-A mismatch at the target site.
  RAB7A_3′UTR_TAG#1_111p56_LEAPER (1)..(111): Used in figure #17, #20, #21
gtctttgataaaaggcgtacataattcttgtgtctactgtacagaatactgccgccagctggatttcccaattctgagtaacactctgcaatccaaacagggttcaaccct

SEQ ID NO: 62 - DNA, 111 bp, Artificial Sequence
  Simple and unstructured LEAPER gRNA that is reverse complementary to the target mRNA, but contains a C-A mismatch at the target site.
  RAB7A_3′UTR_TAG#1_111p56_LEAPER_AG20 (1)..(111): Used in figure #17
gtctttgagaaaaggcggacagaagtctggtgtcgacgggacagaagacggccgccagctggagggcccaaggcggaggaacactcggcaatccaaacagggggcaaccct

SEQ ID NO: 63 - DNA, 111 bp, Artificial Sequence
  Simple and unstructured LEAPER gRNA that is reverse complementary to the target mRNA, but contains a C-A mismatch at the target site.
  NUP43_V233V_111p56_LEAPER (1)..(111): Used in figure #17
tagtaccttgtctaacatcccaaatactcaacattccatcttggccaccagtagccacaacatgctgttggttgggatgtctatcaacacagtggagtggcactcggtcac

SEQ ID NO: 64 - DNA, 111 bp, Artificial Sequence
  Simple and unstructured LEAPER gRNA that is reverse complementary to the target mRNA, but contains a C-A mismatch at the target site.
  NUP43_V233V_111p56_LEAPER_AG6 (1)..(111): Used in figure #17
tagtaccttgtctaacatcccaaagactcaacattccatcgtggccaccaggagccacaacatgctggtgggtgggatgtcgatcaacacagtggagtggcactcggtcac

SEQ ID NO: 65 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_C (1)..(129): Used in figure #12
ggtgtcgagaagaggagaacaatatgctaaatgttgttctcgtctcctcgacacccagtagccacaacatgctgtaaaaccaaccccgtcaagaaaaatcagggatctcccaaattacaaatcaacact

SEQ ID NO: 66 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_D (1)..(129): Used in figure #12
ggtgtcgagaagaggagaacaatatgctaaatgttgttctcgtctcctcgacacccagtagccacaacatgctgtaaagggctcaaaagagccaaattctcaccgcagataaaactcagccaagaaaca

SEQ ID NO: 67 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_E (1)..(129): Used in figure #12
ggtgtcgagaagaggagaacaatatgctaaatgttgttctcgtctcctcgacacccagtagccacaacatgctgtaaagacgtcaaaatcaaaaaattcccaagaaaatctaaattcaaaggcaactct

SEQ ID NO: 68 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_F (1)..(129): Used in figure #12
ggtgtcgagaagaggagaacaatatgctaaatgttgttctcgtctcctcgacacccagtagccacaacatgctgtaaaaccaaccccgtcaagaaaccaccacacccagccaaaaggcaggagaatcac

SEQ ID NO: 69 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_G (1)..(129): Used in figure #12
ggtgtcgagaagaggagaacaatatgctaaatgttgttctcgtctcctcgacacccagtagccacaacatgctgtaaactcagccaagaaacaaaaaacaaaacacacacaaaacagagatcgcaccac

SEQ ID NO: 70 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_H (1)..(129): Used in figure #12
ggtgtcgagaagaggagaacaatatgctaaatgttgttctcgtctcctcgacacccagtagccacaacatgctgtaaatcaccagtcagtgacaaattgtctatggttcttaaaagagtctggttattt

SEQ ID NO: 71 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_B (1)..(129): Used in figure #12
ggtgtcgagaagaggagaacaatatgctaaatgttgttctcgtctcctcgacacccagtagccacaacatgctgtaaagacgtcaaaatcaaaaaaaacacaggaaacacgaaagagacaaggcctcac

SEQ ID NO: 72 - DNA, 129 bp, Artificial Sequence
  R/G-V21 motif, targeting sequence, recruitment sequences & linker.
  NUP43_V223V_RG-V21_3xRS(15-15-15-20p8)_3xA_Placement_A (1)..(129): Used in figure #12
ggtgtcgagaagaggagaacaatatgctaaatgttgttctcgtctcctcgacacccagtagccacaacatgctgtaaaaccatcctctccaacaaaaaaccctccatcagaaaacgaaagcggccgcag

SEQ ID NO: 73 - DNA, 221 bp, Artificial Sequence
  R/G-V21 motif, targeting sequence, recruitment sequences & linker.
  hIDUA_W402X_RG-V21_9xRS_3xA (1)..(221)
ggtgtcgagaagaggagaacaatatgctaaatgttgttctcgtctcctcgacaccttcggcccagagctgctcctaaactcaggaaggcaaaagtcacgtccgccctccaaaaccagcccaccagcgaaacgcccgcctccccagaaatcccagccgcaaaacagaccctccgagaaaggagaccaagtccaaatcctcaaagtcagaaaacaagctccag

SEQ ID NO: 74 - DNA, 129 bp, Artificial Sequence
  R/G-V21 motif, targeting sequence, recruitment sequences & linker.
  hIDUA_W402X_RG-V21_3xRS(15-15-15-20p8)_3xA_Version #1 (1)..(129)
ggtgtcgagaagaggagaacaatatgctaaatgttgttctcgtctcctcgacaccttcggcccagagctgctcctaaaacgtccgccctccacaaaccagccgcacgcccgaaagcgcgcagaccctcc

SEQ ID NO: 75 - DNA, 147 bp, Artificial Sequence
  R/G-V21 motif, targeting sequence, recruitment sequences & linker.
  hIDUA_W402X_RG-V21_4xRS(15-15-15-15-20p8)_3xA_Version #2 (1)..(147)
ggtgtcgagaagaggagaacaatatgctaaatgttgttctcgtctcctcgacaccttcggcccagagctgctcctaaaacgtccgccctccacaaaccagccgcacgcccgaaactcccagccgcagggaaagcgcgcagaccctcc

SEQ ID NO: 76 - DNA, 134 bp, Artificial Sequence
  R/G-V21 motif, targeting sequence, recruitment sequences & linker.
  hIDUA_W402X_RG-V21_3xRS(20-15-15-20p8)_3xA_Version #3 (1)..(134)
ggtgtcgagaagaggagaacaatatgctaaatgttgttctcgtctcctcgacaccttcggcccagagctgctcctaaagcgccagcagccccaaaaagctcaggaaggcataaaaccagcgggtccgcctcgtc

SEQ ID NO: 77 - DNA, 134 bp, Artificial Sequence
  R/G-V21 motif, targeting sequence, recruitment sequences & linker.
  hIDUA_W402X_RG-V21_3xRS(20-15-15-20p8)_3xA_Version #4 (1)..(134)
ggtgtcgagaagaggagaacaatatgctaaatgttgttctcgtctcctcgacaccttcggcccagagctgctcctaaagcgccagcagccccaaaagaccagcccaccagcaaaccgcacgcccgcctccccag

SEQ ID NO: 78 - DNA, 134 bp, Artificial Sequence
  R/G-V21 motif, targeting sequence, recruitment sequences & linker.
  hIDUA_W402X_RG-V21_3xRS(20-15-15-20p8)_3xA_Version #5 (1)..(134)
ggtgtcgagaagaggagaacaatatgctaaatgttgttctcgtctcctcgacaccttcggcccagagctgctcctaaaacgtccgccctccacaaaccagccgcacgcccgaaagaccctccgagcaggcatcg

SEQ ID NO: 79 - DNA, 152 bp, Artificial Sequence
  R/G-V21 motif, targeting sequence, recruitment sequences & linker.
  hIDUA_W402X_RG-V21_4xRS(20-15-15-15-20p8)_3xA_Version #6 (1)..(152)
ggtgtcgagaagaggagaacaatatgctaaatgttgttctcgtctcctcgacaccttcggcccagagctgctcctaaagcgccagcagccccaaaatccgccctccacggcaaagaccagcccaccagcaaaccgcacgcccgcctccccag

SEQ ID NO: 80 - DNA, 144 bp, Artificial Sequence
  R/G-V21 motif, targeting sequence, recruitment sequences & linker.
  hIDUA_W402X_RG-V21_3xRS(20-20-20-20p8)_3xA_Version #7 (1)..(144)
ggtgtcgagaagaggagaacaatatgctaaatgttgttctcgtctcctcgacaccttcggcccagagctgctcctaaatcacgtccgccctccacggcaaaaccagcccaccagcgggtccaaagccgcacgcccgcctcccca

SEQ ID NO: 81 - DNA, 138 bp, Artificial Sequence
  R/G-V21 motif, targeting sequence, recruitment sequences & linker.
  hIDUA_W402X_RG-V21_3xRS(26-14-14-20p8)_3xA_Version #8 (1)..(138)
ggtgtcgagaagaggagaacaatatgctaaatgttgttctcgtctcctcgacaccttcggcccagagctgctcctaaaagctcaggaaggcaaaagaccagcccaccagaaagtccagccgcacgcccgcctccccag

SEQ ID NO: 82 - DNA, 129 bp, Artificial Sequence
  R/G-V21 motif, targeting sequence, recruitment sequences & linker.
  hIDUA_W402X_RG-V21_3xRS(15-15-15-20p8)_3xA_Version #9 (1)..(129)
ggtgtcgagaagaggagaacaatatgctaaatgttgttctcgtctcctcgacaccttcggcccagagctgctcctaaagcgccagcagccccaaaatgcggcgggcgggtgaaatggaagcgcgcggtg

SEQ ID NO: 83 - DNA, 134 bp, Artificial Sequence
  R/G-V21 motif, targeting sequence, recruitment sequences & linker.
  hIDUA_W402X_RG-V21_3xRS(20-15-15-20p8)_3xA_Version #10 (1)..(134)
ctggtgggctggtccctgcctttcaccgcgcgcttccatttcacccgcccgccgcatttaggagcagctctgggccgaaggtgtcgaggagacgagaacaacatttagcatattgttctcctcttctcgacacc

SEQ ID NO: 84 - DNA, 21 bp, Artificial Sequence
  DNA Oligo
  Fluc_W417X_Universal_fw (1)..(21)
acttggacaccggtaagacac

SEQ ID NO: 85 - DNA, 21 bp, Artificial Sequence
  DNA Oligo
  Fluc_W417X_Universal_bw (1)..(21)
acgatgaagaagtgctcgtcc

SEQ ID NO: 86 - DNA, 18 bp, Artificial Sequence
  DNA Oligo
  Fluc_W417X_Nested_fw (1)..(18)
tgggtgtgaaccagcgcg

SEQ ID NO: 87 - DNA, 19 bp, Artificial Sequence
  DNA Oligo
  Fluc_W417X_Nested_bw (1)..(19)
gtcccagtaggcgatgtcg

SEQ ID NO: 88 - DNA, 8062 bp, Artificial Sequence
  Plasmid containing a 2A peptide interrupted dual-luciferase (firefly, renilla) construct behind a CMV promoter. Both luciferases are functional.
  Renilla-Luciferase_wt (906)..(1838)
  2A-peptide (1839)..(1904)
  Firefly-Luciferase_wt (1905)..(3557)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttctagataagatatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaaggaagcggagctactaacttcagcctgctgaagcaggctggagacgtggaggagaaccctggacctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggtcacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 89 - DNA, 8062 bp, Artificial Sequence
  Plasmid containing a 2A peptide interrupted dual-luciferase (firefly, renilla) construct behind a CMV promoter. The renilla luciferases is functional. The firefly luciferase contains a W417X amber mutation.
  Renilla-Luciferase_wt (906)..(1838)
  2A-peptide (1839)..(1904)
  Firefly-Luciferase_W417X_amber (1905)..(3557)
  G-to-A_amber_mutation (3154)..(3154)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttctagataagatatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaaggaagcggagctactaacttcagcctgctgaagcaggctggagacgtggaggagaaccctggacctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctagctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggtcacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 90 - DNA, 129 bp, Artificial Sequence
  R/G-V21 motif, targeting sequence, recruitment sequences & linker.
  Luciferase_W417X_RG-V21_3xRS(15-15-20p8-15)_3xA (1)..(129): Used in figure #18
ggtgtcgagaagaggagaacaatatgctaaatgttgttctcgtctcctcgacacctcgtcctcgtcccagaaagtgcagccagccgtccttgtaaagtccaagtccaccacaaaacgcacagctcgccg

SEQ ID NO: 91 - DNA, 129 bp, Artificial Sequence
  R/G-V21 motif, targeting sequence, recruitment sequences & linker.
  Luciferase_W417X_RG-V21_3xRS(15-20p8-15-15)_3xA (1)..(129): Used in figure #18
ggtgtcgagaagaggagaacaatatgctaaatgttgttctcgtctcctcgacacctcagccggtccacgaaaatcgtcctcgtcccagaaagtgcagccagccgtccttgtaaagtccaagtccaccac

SEQ ID NO: 92 - DNA, 149 bp, Artificial Sequence
  R/G-V21 motif, targeting sequence, recruitment sequences & linker.
  misc-feature (1)..(3): chemical modification at the 2′ carbon atom with 2′-O-methyl
  misc-feature (147)..(149): chemical modification at the 2′ carbon atom with 2′-O-methyl
  misc-feature (1)..(3): phosphorothioate linkage
  misc-feature (124)..(149): phosphorothioate linkage
  hIDUA_W402X_RG-V21_3xRS(20-20p8-25-20)_3xA_ASO (1)..(149): Used in figure #19
ggggucgagaagaggagaacaauaugcuaaauguuguucucgucuccucgacaccgccaggacgcccaccgugugaaacuguccaggacggucccggccugcgaaauucggcccagagcugcuccuaaaguggggucguugcccagccg